SEQUENCE LISTING (395 sequences)

SEQ ID NO: 1 - protein, 128 aa, Artificial Sequence
  Description of Artificial Sequence Synthetic polypeptide
QVQLVQSGAEVKKPGASVKVSCKASGYTFTNYHLHWVRQAPGQGLEWMGMINPNDGSTTYAQKFQGRVTMTRDTSTSTVYMELSSLRSEDTAVYYCARSTYYYDSSGYYYYYYGMDVWGQGTTVTVSS

SEQ ID NO: 2 - protein, 9 aa, Artificial Sequence
  Description of Artificial Sequence Synthetic peptide
YTFTNYHLH

SEQ ID NO: 3 - protein, 13 aa, Artificial Sequence
  Description of Artificial Sequence Synthetic peptide
GMINPNDGSTTYA

SEQ ID NO: 4 - protein, 23 aa, Artificial Sequence
  Description of Artificial Sequence Synthetic peptide
CARSTYYYDSSGYYYYYYGMDVW

SEQ ID NO: 5 - protein, 108 aa, Artificial Sequence
  Description of Artificial Sequence Synthetic polypeptide
EIVMTQSPATLSVSPGERATLSCRASQSVYSNYLAWYQQKPGQAPRLLIYGASTRATGIPARFSGSGSGTEFTLTISSLQSEDFAVYYCQQYHSYPLTFGGGTKVEIK

SEQ ID NO: 6 - protein, 12 aa, Artificial Sequence
  Description of Artificial Sequence Synthetic peptide
RASQSVYSNYLA

SEQ ID NO: 7 - protein, 7 aa, Artificial Sequence
  Description of Artificial Sequence Synthetic peptide
GASTRAT

SEQ ID NO: 8 - protein, 11 aa, Artificial Sequence [sequence omitted]

SEQ ID NO: 9 - protein, 115 aa, Artificial Sequence
  Description of Artificial Sequence Synthetic polypeptide
QVQLVQSGAEVKKPGSSVKVSCKASGYRFTGYHLHWVRQAPGQGLEWMGGIIPIFGTANYAQKFQGRVTITADKSTSTAYMELSSLRSEDTAVYYCAYGVPPDPWGQGTLVTVSS

SEQ ID NO: 10 - protein, 9 aa, Artificial Sequence
  Description of Artificial Sequence Synthetic peptide
YRFTGYHLH

SEQ ID NO: 11 - protein, 13 aa, Artificial Sequence
  Description of Artificial Sequence Synthetic peptide
GGIIPIFGTANYA

SEQ ID NO: 12 - protein, 10 aa, Artificial Sequence
  Description of Artificial Sequence Synthetic peptide
CAYGVPPDPW

SEQ ID NO: 13 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSYRASGVPDRFSGSGSGTDFTLKISRVEAEDVGVYYCRQALRTPLTFGGGTKVEIK

SEQ ID NO: 14 - protein, 16 aa, Artificial Sequence
  Description of Artificial Sequence Synthetic peptide
RSSQSLLHSNGYNYLD

SEQ ID NO: 15 - protein, 7 aa, Artificial Sequence
  Description of Artificial Sequence Synthetic peptide
LGSYRAS

SEQ ID NO: 16 - protein, 11 aa, Artificial Sequence
  Description of Artificial Sequence Synthetic peptide
CRQALRTPLTF

SEQ ID NO: 17 - protein, 121 aa, Artificial Sequence
  Description of Artificial Sequence Synthetic polypeptide
QVQLVQSGAEVKKPGASVKVSCKASGYTFTSYDINWVRQAPGQGLEWMGWISGYNGNTNYAQKFQGRVTMTRDTSTSTVYMELSSLRSEDTAVYYCARSHEYYYYYGMDVWGQGTLVTVSS

SEQ ID NO: 18 - protein, 9 aa, Artificial Sequence
  Description of Artificial Sequence Synthetic peptide
YTFTSYDIN

SEQ ID NO: 19 - protein, 13 aa, Artificial Sequence
  Description of Artificial Sequence Synthetic peptide
GWISGYNGNTNYA

SEQ ID NO: 20 - protein, 16 aa, Artificial Sequence
  Description of Artificial Sequence Synthetic peptide
CARSHEYYYYYGMDVW

SEQ ID NO: 21 - protein, 108 aa, Artificial Sequence
  Description of Artificial Sequence Synthetic polypeptide
EIVMTQSPATLSVSPGERATLSCRASQSVSSSYLAWYQQKPGQAPRLLIYGASTRATGIPARFSGSGSGTEFTLTISSLQSEDFAVYYCQQYYTTPFTFGPGTKVDIK

SEQ ID NO: 22 - protein, 12 aa, Artificial Sequence
  Description of Artificial Sequence Synthetic peptide
RASQSVSSSYLA

SEQ ID NO: 23 - protein, 7 aa, Artificial Sequence
  Description of Artificial Sequence Synthetic peptide
GASTRAT

SEQ ID NO: 24 - protein, 11 aa, Artificial Sequence [sequence omitted]

SEQ ID NO: 25 - protein, 129 aa, Artificial Sequence
  Description of Artificial Sequence Synthetic polypeptide
EVQLLESGGGLVQPGGSLRLSCAASGFTFSSYDMHWVRQAPGKGLEWVSAISSSGGSIYYADSVKGRFTISRDNSKNTLYLQMNSLRAEDTAVYYCARDSVVVPKGPNRKYYYYGMDVWGQGTKVTVSS

SEQ ID NO: 26 - protein, 9 aa, Artificial Sequence
  Description of Artificial Sequence Synthetic peptide
FTFSSYDMH

SEQ ID NO: 27 - protein, 13 aa, Artificial Sequence
  Description of Artificial Sequence Synthetic peptide
SAISSSGGSIYYA

SEQ ID NO: 28 - protein, 24 aa, Artificial Sequence
  Description of Artificial Sequence Synthetic peptide
CARDSVVVPKGPNRKYYYYGMDVW

SEQ ID NO: 29 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQATHSPYTFGQGTKVEIK

SEQ ID NO: 30 - protein, 16 aa, Artificial Sequence
  Description of Artificial Sequence Synthetic peptide
RSSQSLLHSNGYNYLD

SEQ ID NO: 31 - protein, 7 aa, Artificial Sequence
  Description of Artificial Sequence Synthetic peptide
LGSNRAS

SEQ ID NO: 32 - protein, 11 aa, Artificial Sequence [sequence omitted]

SEQ ID NO: 33 - protein, 126 aa, Artificial Sequence
  Description of Artificial Sequence Synthetic polypeptide
EVQLLESGGGLVQPGGSLRLSCAASGFTFDAYAMHWVRQAPGKGLEWVSAIGTGGDTYYADSVKGRFTISRDNSKNTLYLQMNSLRAEDTAVYYCARDLYGSGSPQYYYYYGMDVWGQGTTVTVSS

SEQ ID NO: 34 - protein, 9 aa, Artificial Sequence
  Description of Artificial Sequence Synthetic peptide
FTFDAYAMH

SEQ ID NO: 35 - protein, 12 aa, Artificial Sequence
  Description of Artificial Sequence Synthetic peptide
SAIGTGGDTYYA

SEQ ID NO: 36 - protein, 22 aa, Artificial Sequence
  Description of Artificial Sequence Synthetic peptide
CARDLYGSGSPQYYYYYGMDVW

SEQ ID NO: 37 - protein, 107 aa, Artificial Sequence
  Description of Artificial Sequence Synthetic polypeptide
DIQMTQSPSSLSASVGDRVTITCRASQGIKNDLGWYQQKPGKAPKLLIYAASSLQSGVPSRFSGSGSGTDFTLTISSLQPEDFATYYCQQSYSVPFTFGQGTKVEIK

SEQ ID NO: 38 - protein, 11 aa, Artificial Sequence
  Description of Artificial Sequence Synthetic peptide
RASQGIKNDLG

SEQ ID NO: 39 - protein, 7 aa, Artificial Sequence
  Description of Artificial Sequence Synthetic peptide
AASSLQS

SEQ ID NO: 40 - protein, 11 aa, Artificial Sequence [sequence omitted]

SEQ ID NO: 41 - protein, 121 aa, Artificial Sequence
  Description of Artificial Sequence Synthetic polypeptide
QVQLVQSGAEVKKPGASVKVSCKASGYAFTAYYLHWVRQAPGQGLEWMGWMNPNSGNTGYAQKFQGRVTMTRDTSTSTVYMELSSLRSEDTAVYYCAREGWGYYDGGFDPWGQGTLVTVSS

SEQ ID NO: 42 - protein, 9 aa, Artificial Sequence
  Description of Artificial Sequence Synthetic peptide
YAFTAYYLH

SEQ ID NO: 43 - protein, 13 aa, Artificial Sequence
  Description of Artificial Sequence Synthetic peptide
GWMNPNSGNTGYA

SEQ ID NO: 44 - protein, 16 aa, Artificial Sequence
  Description of Artificial Sequence Synthetic peptide
CAREGWGYYDGGFDPW

SEQ ID NO: 45 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSRRASGVPDRFSGSGSGTDFTLKISRVEAEDVGVYYCMQGSHWPPTFGPGTKVDIK

SEQ ID NO: 46 - protein, 16 aa, Artificial Sequence
  Description of Artificial Sequence Synthetic peptide
RSSQSLLHSNGYNYLD

SEQ ID NO: 47 - protein, 7 aa, Artificial Sequence
  Description of Artificial Sequence Synthetic peptide
LGSRRAS

SEQ ID NO: 48 - protein, 11 aa, Artificial Sequence [sequence omitted]

SEQ ID NO: 49 - protein, 128 aa, Artificial Sequence
  Description of Artificial Sequence Synthetic polypeptide
QVQLVQSGAEVKKPGASVKVSCKASGGTSSTYAVTWVRQAPGQGLEWMGVINPNDGSTTYAQNFQGRVTMTRDTSTSTVYMELSSLRSEDTAVYYCARWTPLFGVVIPDYYYYGMDVWGQGTLVTVSS

SEQ ID NO: 50 - protein, 9 aa, Artificial Sequence
  Description of Artificial Sequence Synthetic peptide
GTSSTYAVT

SEQ ID NO: 51 - protein, 13 aa, Artificial Sequence
  Description of Artificial Sequence Synthetic peptide
GVINPNDGSTTYA

SEQ ID NO: 52 - protein, 23 aa, Artificial Sequence
  Description of Artificial Sequence Synthetic peptide
CARWTPLFGVVIPDYYYYGMDVW

SEQ ID NO: 53 - protein, 107 aa, Artificial Sequence
  Description of Artificial Sequence Synthetic polypeptide
DIQMTQSPSSLSASVGDRVTITCQASQDISNYLNWYQQKPGKAPKLLIYAASSLQSGVPSRFSGSGSGTDFTLTISSLQPEDFATYYCQQSYSTPPTFGQGTRLEIK

SEQ ID NO: 54 - protein, 11 aa, Artificial Sequence
  Description of Artificial Sequence Synthetic peptide
QASQDISNYLN

SEQ ID NO: 55 - protein, 7 aa, Artificial Sequence
  Description of Artificial Sequence Synthetic peptide
AASSLQS

SEQ ID NO: 56 - protein, 11 aa, Artificial Sequence
  Description of Artificial Sequence Synthetic peptide
CQQSYSTPPTF

SEQ ID NO: 57 - protein, 115 aa, Artificial Sequence
  Description of Artificial Sequence Synthetic polypeptide
QVQLVQSGAEVKKPGASVKVSCKASGGTFSSYVISWVRQAPGQGLEWMGWMNPGSGNTGYAQKFQGRVTMTRDTSTSTVYMELSSLRSEDTAVYYCITDSEDDYWGQGTLVTVSS

SEQ ID NO: 58 - protein, 9 aa, Artificial Sequence
  Description of Artificial Sequence Synthetic peptide
GTFSSYVIS

SEQ ID NO: 59 - protein, 13 aa, Artificial Sequence
  Description of Artificial Sequence Synthetic peptide
GWMNPGSGNTGYA

SEQ ID NO: 60 - protein, 10 aa, Artificial Sequence
  Description of Artificial Sequence Synthetic peptide
CITDSEDDYW

SEQ ID NO: 61 - protein, 113 aa, Artificial Sequence
  Description of Artificial Sequence Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYNTPYTFGQGTKVEIK

SEQ ID NO: 62 - protein, 17 aa, Artificial Sequence
  Description of Artificial Sequence Synthetic peptide
KSSQSVLYSSNNKNYLA

SEQ ID NO: 63 - protein, 7 aa, Artificial Sequence
  Description of Artificial Sequence Synthetic peptide
WASTRES

SEQ ID NO: 64 - protein, 11 aa, Artificial Sequence [sequence omitted]

SEQ ID NO: 65 - protein, 128 aa, Artificial Sequence
  Description of Artificial Sequence Synthetic polypeptide
QVQLVQSGAEVKKPGASVKVSCKASGHTFTSQYMHWVRQAPGQGLEWVGVINPNDGSTSYAQKFQGRVTMTRDTSTSTVYMELSSLRSEDTAVYYCARDGAYYYDSSGYYRSSNFDYWGQGTLVTVSS

SEQ ID NO: 66 - protein, 9 aa, Artificial Sequence
  Description of Artificial Sequence Synthetic peptide
HTFTSQYMH

SEQ ID NO: 67 - protein, 13 aa, Artificial Sequence
  Description of Artificial Sequence Synthetic peptide
GVINPNDGSTSYA

SEQ ID NO: 68 - protein, 23 aa, Artificial Sequence
  Description of Artificial Sequence Synthetic peptide
CARDGAYYYDSSGYYRSSNFDYW

SEQ ID NO: 69 - protein, 107 aa, Artificial Sequence
  Description of Artificial Sequence Synthetic polypeptide
DIQMTQSPSSLSASVGDRVTITCQASQDIGNYLNWYQQKPGKAPKLLIYGASNLETGVPSRFSGSGSGTDFTLTISSLQPEDFATYYCQQGYSFPLTFGQGTKVEIK

SEQ ID NO: 70 - protein, 11 aa, Artificial Sequence
  Description of Artificial Sequence Synthetic peptide
QASQDIGNYLN

SEQ ID NO: 71 - protein, 7 aa, Artificial Sequence
  Description of Artificial Sequence Synthetic peptide
GASNLET

SEQ ID NO: 72 - protein, 11 aa, Artificial Sequence [sequence omitted]

SEQ ID NO: 73 - protein, 119 aa, Artificial Sequence
  Description of Artificial Sequence Synthetic polypeptide
QVQLVQSGAEVKKPGASVKVSCKASGYTFTSNYMHWVRQAPGQGLEWMGWMNPNSGNTAYAQKFQGRVTMTRDTSTSTVYMELSSLRSEDTAVYYCARGRGGQLLFDYWGQGTLVTVSS

SEQ ID NO: 74 - protein, 9 aa, Artificial Sequence
  Description of Artificial Sequence Synthetic peptide
YTFTSNYMH

SEQ ID NO: 75 - protein, 13 aa, Artificial Sequence
  Description of Artificial Sequence Synthetic peptide
GWMNPNSGNTAYA

SEQ ID NO: 76 - protein, 14 aa, Artificial Sequence
  Description of Artificial Sequence Synthetic peptide
CARGRGGQLLFDYW

SEQ ID NO: 77 - protein, 106 aa, Artificial Sequence
  Description of Artificial Sequence Synthetic polypeptide
DIQMTQSPSSLSASVGDRVTITCRASQGISSWLAWYQQKPGKAPKLLIYAASTLQSGVPSRFSGSGSGTDFTLTISSLQPEDFATYYCQQYNSYSTFGQGTKVEIK

SEQ ID NO: 78 - protein, 11 aa, Artificial Sequence
  Description of Artificial Sequence Synthetic peptide
RASQGISSWLA

SEQ ID NO: 79 - protein, 7 aa, Artificial Sequence
  Description of Artificial Sequence Synthetic peptide
AASTLQS

SEQ ID NO: 80 - protein, 10 aa, Artificial Sequence [sequence omitted]

SEQ ID NO: 81 - protein, 121 aa, Artificial Sequence
  Description of Artificial Sequence Synthetic polypeptide
QVQLVQSGAEVKKPGASVKVSCKASGGTFSNYGISWVRQAPGQGLEWMGIINPTDGSTTYAQKFQGRVTMTRDTSTSTVYMELSSLRSEDTAVYYCARWWGSGWSWYFDLWGRGTLVTVSS

SEQ ID NO: 82 - protein, 9 aa, Artificial Sequence
  Description of Artificial Sequence Synthetic peptide
GTFSNYGIS

SEQ ID NO: 83 - protein, 13 aa, Artificial Sequence
  Description of Artificial Sequence Synthetic peptide
GIINPTDGSTTYA

SEQ ID NO: 84 - protein, 16 aa, Artificial Sequence
  Description of Artificial Sequence Synthetic peptide
CARWWGSGWSWYFDLW

SEQ ID NO: 85 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASSLESGVPSRFSGSGSGTDFTLTISSLQPEDFATYYCQQSYSTPPTFGQSTRLEIK

SEQ ID NO: 86 - protein, 11 aa, Artificial Sequence
  Description of Artificial Sequence Synthetic peptide
RASQSISSYLN

SEQ ID NO: 87 - protein, 7 aa, Artificial Sequence
  Description of Artificial Sequence Synthetic peptide
AASSLES

SEQ ID NO: 88 - protein, 11 aa, Artificial Sequence
  Description of Artificial Sequence Synthetic peptide
CQQSYSTPPTF

SEQ ID NO: 89 - protein, 116 aa, Artificial Sequence
  Description of Artificial Sequence Synthetic polypeptide
QVQLVQSGAEVKKPGASVKVSCKASGYTFTSYAINWVRQAPGQGLEWMGILSPSGGGTSYAPKFQGRVTMTRDTSTSTVYMELSSLRSEDTAVYYCARGPWYFDLWGRGTLVTVSS

SEQ ID NO: 90 - protein, 9 aa, Artificial Sequence
  Description of Artificial Sequence Synthetic peptide
YTFTSYAIN

SEQ ID NO: 91 - protein, 13 aa, Artificial Sequence
  Description of Artificial Sequence Synthetic peptide
GILSPSGGGTSYA

SEQ ID NO: 92 - protein, 11 aa, Artificial Sequence
  Description of Artificial Sequence Synthetic peptide
CARGPWYFDLW

SEQ ID NO: 93 - protein, 107 aa, Artificial Sequence
  Description of Artificial Sequence Synthetic polypeptide
DIQMTQSPSSLSASVGDRVTITCRASQGISNSLAWYQQKPGKAPKLLIYAASSLQSGVPSRFSGSGSGTDFTLTISSLQPEDFATYYCQQSYSTPFTFGPGTKVDIK

SEQ ID NO: 94 - protein, 11 aa, Artificial Sequence
  Description of Artificial Sequence Synthetic peptide
RASQGISNSLA

SEQ ID NO: 95 - protein, 7 aa, Artificial Sequence
  Description of Artificial Sequence Synthetic peptide
AASSLQS

SEQ ID NO: 96 - protein, 11 aa, Artificial Sequence [sequence omitted]

SEQ ID NO: 97 - protein, 119 aa, Artificial Sequence
  Description of Artificial Sequence Synthetic polypeptide
QVQLVQSGAEVKKPGSSVKVSCKASGGTFSSYAISWVRQAPGQGLEWMGGIVPMLGSPHYAQKFQGRVTITADESTSTAYMELSSLRSEDTAVYYCARGSWLVADFQHWGQGTLVTVSS

SEQ ID NO: 98 - protein, 9 aa, Artificial Sequence
  Description of Artificial Sequence Synthetic peptide
GTFSSYAIS

SEQ ID NO: 99 - protein, 13 aa, Artificial Sequence
  Description of Artificial Sequence Synthetic peptide
GGIVPMLGSPHYA

SEQ ID NO: 100 - protein, 14 aa, Artificial Sequence
  Description of Artificial Sequence Synthetic peptide
CARGSWLVADFQHW

SEQ ID NO: 101 - protein, 113 aa, Artificial Sequence
  Description of Artificial Sequence Synthetic polypeptide
DIVMTQSPDSLAVSLGERATINCKSSQSLLYSSNNRNYLAWYQQKPGQPPKLLIYWASTRESGVPDRFSGSGSGTDFTLTISSLQAEDVAVYYCQQYYSTPITFGQGTRLEIK

SEQ ID NO: 102 - protein, 17 aa, Artificial Sequence
  Description of Artificial Sequence Synthetic peptide
KSSQSLLYSSNNRNYLA

SEQ ID NO: 103 - protein, 7 aa, Artificial Sequence
  Description of Artificial Sequence Synthetic peptide
WASTRES

SEQ ID NO: 104 - protein, 11 aa, Artificial Sequence [sequence omitted]

SEQ ID NO: 105 - protein, 121 aa, Artificial Sequence
  Description of Artificial Sequence Synthetic polypeptide
EVQLLESGGGLVKPGGSLRLSCAASGFRFSVYWMSWVRQAPGKGLEWVSGISGSGGTTYYADSVKGRFTISRDDSKNTLYLQMNSLKTEDTAVYYCARVRRDGYNYNFDYWGQGTLVTVSS

SEQ ID NO: 106 - protein, 9 aa, Artificial Sequence
  Description of Artificial Sequence Synthetic peptide
FRFSVYWMS

SEQ ID NO: 107 - protein, 13 aa, Artificial Sequence
  Description of Artificial Sequence Synthetic peptide
SGISGSGGTTYYA

SEQ ID NO: 108 - protein, 16 aa, Artificial Sequence
  Description of Artificial Sequence Synthetic peptide
CARVRRDGYNYNFDYW

SEQ ID NO: 109 - protein, 107 aa, Artificial Sequence
  Description of Artificial Sequence Synthetic polypeptide
DIQMTQSPSSLSASVGDRVTITCQASQDISNYLNWYQQKPGKAPKLLIFDASSLEAGVPSRFSGSGSGTDFTLTISSLQPEDFATYYCQQANSFPPTFGQGTEVEIK

SEQ ID NO: 110 - protein, 11 aa, Artificial Sequence
  Description of Artificial Sequence Synthetic peptide
QASQDISNYLN

SEQ ID NO: 111 - protein, 7 aa, Artificial Sequence
  Description of Artificial Sequence Synthetic peptide
DASSLEA

SEQ ID NO: 112 - protein, 11 aa, Artificial Sequence [sequence omitted]

SEQ ID NO: 113 - protein, 124 aa, Artificial Sequence
  Description of Artificial Sequence Synthetic polypeptide
QVQLVQSGAEVKKPGSSVKVSCKASGFTFTTSAVQWVRQAPGQGLEWMGGIIPIFGTANYAQKFQGRVTITADESTSTAYMELSSLRSEDTAVYYCAKGSGYEFPGGSEYFQHWGQGTLVTVSS

SEQ ID NO: 114 - protein, 9 aa, Artificial Sequence
  Description of Artificial Sequence Synthetic peptide
FTFTTSAVQ

SEQ ID NO: 115 - protein, 13 aa, Artificial Sequence
  Description of Artificial Sequence Synthetic peptide
GGIIPIFGTANYA

SEQ ID NO: 116 - protein, 19 aa, Artificial Sequence
  Description of Artificial Sequence Synthetic peptide
CAKGSGYEFPGGSEYFQHW

SEQ ID NO: 117 - protein, 113 aa, Artificial Sequence
  Description of Artificial Sequence Synthetic polypeptide
DIVMTQSPDSLAVSLGERATINCKSSQSVLYNSNNLNYLAWYQQKPGQPPKLLIYWASIRESGVPDRFSGSGSGTDFTLTISSLQAEDVAVYYCQQYYSTPLTFGQGTKVEIK

SEQ ID NO: 118 - protein, 17 aa, Artificial Sequence
  Description of Artificial Sequence Synthetic peptide
KSSQSVLYNSNNLNYLA

SEQ ID NO: 119 - protein, 7 aa, Artificial Sequence
  Description of Artificial Sequence Synthetic peptide
WASIRES

SEQ ID NO: 120 - protein, 11 aa, Artificial Sequence [sequence omitted]

SEQ ID NO: 121 - protein, 129 aa, Artificial Sequence
  Description of Artificial Sequence Synthetic polypeptide
QVQLVQSGAEVKKPGASVKVSCKASGGTLSSYAISWVRQAPGQGLEWMGWIDPNSGGTNYAQKFQGRVTMTRDTSTSTVYMELSSLRSEDTAVYYCARQGLLWFGESGSIYYYYGMDVWGQGTTVTVSS

SEQ ID NO: 122 - protein, 9 aa, Artificial Sequence
  Description of Artificial Sequence Synthetic peptide
GTLSSYAIS

SEQ ID NO: 123 - protein, 13 aa, Artificial Sequence
  Description of Artificial Sequence Synthetic peptide
GWIDPNSGGTNYA

SEQ ID NO: 124 - protein, 24 aa, Artificial Sequence
  Description of Artificial Sequence Synthetic peptide
CARQGLLWFGESGSIYYYYGMDVW

SEQ ID NO: 125 - protein, 107 aa, Artificial Sequence
  Description of Artificial Sequence Synthetic polypeptide
DIQMTQSPSSLSASVGDRVTITCRASRSISNYLNWYQQKPGKAPKLLIYGASSLQSGVPSRFSGSGSGTDFTLTISSLQPEDFATYYCQQSYSTLVTFGQGTKVEIK

SEQ ID NO: 126 - protein, 11 aa, Artificial Sequence
  Description of Artificial Sequence Synthetic peptide
RASRSISNYLN

SEQ ID NO: 127 - protein, 7 aa, Artificial Sequence
  Description of Artificial Sequence Synthetic peptide
GASSLQS

SEQ ID NO: 128 - protein, 11 aa, Artificial Sequence [sequence omitted]

SEQ ID NO: 129 - protein, 123 aa, Artificial Sequence
  Description of Artificial Sequence Synthetic polypeptide
QVQLVQSGAEVKKPGASVKVSCKASGYTFTSYGISWVRQAPGQGLEWMGWMNPNSGNTGYAQKFQGRVTMTRDTSTSTVYMELSSLRSEDTAVYYCARLGLWFGEYQYYFDYWGQGTLVTVSS

SEQ ID NO: 130 - protein, 9 aa, Artificial Sequence
  Description of Artificial Sequence Synthetic peptide
YTFTSYGIS

SEQ ID NO: 131 - protein, 13 aa, Artificial Sequence
  Description of Artificial Sequence Synthetic peptide
GWMNPNSGNTGYA

SEQ ID NO: 132 - protein, 18 aa, Artificial Sequence
  Description of Artificial Sequence Synthetic peptide
CARLGLWFGEYQYYFDYW

SEQ ID NO: 133 - protein, 113 aa, Artificial Sequence
  Description of Artificial Sequence Synthetic polypeptide
DIVMTQSPDSLAVSLGERATINCKSSQSVLYSSNNKNYLAWYQQKPGQPPKLLIYWASTREPGVPDRFSGSGSGTDFTLTISSLQAEDVAVYYCQQYYSTPLTFGQGTKVEIK

SEQ ID NO: 134 - protein, 17 aa, Artificial Sequence
  Description of Artificial Sequence Synthetic peptide
KSSQSVLYSSNNKNYLA

SEQ ID NO: 135 - protein, 7 aa, Artificial Sequence
  Description of Artificial Sequence Synthetic peptide
WASTREP

SEQ ID NO: 136 - protein, 11 aa, Artificial Sequence [sequence omitted]

SEQ ID NO: 137 - protein, 122 aa, Artificial Sequence
  Description of Artificial Sequence Synthetic polypeptide
EVQLLESGGGLVKPGGSLRLSCAASGFTFSSYWMSWVRQAPGKGLEWVSGISWNGGTVGYADSVKGRFTISRDDSKNTLYLQMNSLKTEDTAVYYCAKLGIAVKSHWYFDLWGRGTLVTVSS

SEQ ID NO: 138 - protein, 9 aa, Artificial Sequence
  Description of Artificial Sequence Synthetic peptide
FTFSSYWMS

SEQ ID NO: 139 - protein, 13 aa, Artificial Sequence
  Description of Artificial Sequence Synthetic peptide
SGISWNGGTVGYA

SEQ ID NO: 140 - protein, 17 aa, Artificial Sequence
  Description of Artificial Sequence Synthetic peptide
CAKLGIAVKSHWYFDLW

SEQ ID NO: 141 - protein, 107 aa, Artificial Sequence
  Description of Artificial Sequence Synthetic polypeptide
DIQMTQSPSSLSASVGDRVTITCRASESISTWLAWYQQKPGKAPKLLIYAASSLQSGVPSRFSGSGSGTDFTLTISSLQPEDFATYYCQQSYTLPLTFGGGTKLEIK

SEQ ID NO: 142 - protein, 11 aa, Artificial Sequence
  Description of Artificial Sequence Synthetic peptide
RASESISTWLA

SEQ ID NO: 143 - protein, 7 aa, Artificial Sequence
  Description of Artificial Sequence Synthetic peptide
AASSLQS

SEQ ID NO: 144 - protein, 11 aa, Artificial Sequence
  Description of Artificial Sequence Synthetic peptide
CQQSYTLPLTF

SEQ ID NO: 145 - protein, 123 aa, Artificial Sequence
  Description of Artificial Sequence Synthetic polypeptide
QVQLVQSGAEVKKPGASVKVSCKASGYTFTSYDINWVRQAPGQGLEWVGIINPSGGSTSYAQKFQGRVTMTRDTSTSTVYMELSSLRSEDTAVYYCARASSGGYYYYYGMDVWGQGTTVTVSS

SEQ ID NO: 146 - protein, 9 aa, Artificial Sequence
  Description of Artificial Sequence Synthetic peptide
YTFTSYDIN

SEQ ID NO: 147 - protein, 13 aa, Artificial Sequence
  Description of Artificial Sequence Synthetic peptide
GIINPSGGSTSYA

SEQ ID NO: 148 - protein, 18 aa, Artificial Sequence
  Description of Artificial Sequence Synthetic peptide
CARASSGGYYYYYGMDVW

SEQ ID NO: 149 - protein, 107 aa, Artificial Sequence
  Description of Artificial Sequence Synthetic polypeptide
DIQMTQSPSSLSASVGDRVTITCRASQDIVNWLAWYQQKPGKAPKLLIYAASSLQSGVPSRFSGSGSGTDFTLTISSLQPEDFATYYCQQYYSYPLTFGQGTRLEIK

SEQ ID NO: 150 - protein, 11 aa, Artificial Sequence
  Description of Artificial Sequence Synthetic peptide
RASQDIVNWLA

SEQ ID NO: 151 - protein, 7 aa, Artificial Sequence
  Description of Artificial Sequence Synthetic peptide
AASSLQS

SEQ ID NO: 152 - protein, 11 aa, Artificial Sequence [sequence omitted]

SEQ ID NO: 153 - protein, 120 aa, Artificial Sequence
  Description of Artificial Sequence Synthetic polypeptide
QVQLVQSGAEVKKPGASVKVSCKASGGTFSNYAISWVRQAPGQGLEWMGVINPRGGSTTYAQKFQGRVTMTRDTSTSTVYMELSSLRSEDTAVYYCARDYSIPYYGMDVWGQGTTVTVSS

SEQ ID NO: 154 - protein, 9 aa, Artificial Sequence
  Description of Artificial Sequence Synthetic peptide
GTFSNYAIS

SEQ ID NO: 155 - protein, 13 aa, Artificial Sequence
  Description of Artificial Sequence Synthetic peptide
GVINPRGGSTTYA

SEQ ID NO: 156 - protein, 15 aa, Artificial Sequence
  Description of Artificial Sequence Synthetic peptide
CARDYSIPYYGMDVW

SEQ ID NO: 157 - protein, 107 aa, Artificial Sequence
  Description of Artificial Sequence Synthetic polypeptide
DIQMTQSPSSLSASVGDRVTITCRASQSISTYLNWYQQKPGKAPKLLIYDASNLETGVPSRFSGSGSGTDFTLTISSLQPEDFATYYCQQSYSTPLTFGGGTKVEIK

SEQ ID NO: 158 - protein, 11 aa, Artificial Sequence
  Description of Artificial Sequence Synthetic peptide
RASQSISTYLN

SEQ ID NO: 159 - protein, 7 aa, Artificial Sequence
  Description of Artificial Sequence Synthetic peptide
DASNLET

SEQ ID NO: 160 - protein, 11 aa, Artificial Sequence [sequence omitted]

SEQ ID NO: 161 - protein, 117 aa, Artificial Sequence
  Description of Artificial Sequence Synthetic polypeptide
EVQLLESGGGLVQPGGSLRLSCAASGFTFSNHYMSWVRQAPGKGLEWVAVIALDGSYRYYADSVKGRFTISRDNSKNTLYLQMNSLRAEDTAVYYCARVGPGGMDVRGQGTTVTVSS

SEQ ID NO: 162 - protein, 9 aa, Artificial Sequence
  Description of Artificial Sequence Synthetic peptide
FTFSNHYMS

SEQ ID NO: 163 - protein, 13 aa, Artificial Sequence
  Description of Artificial Sequence Synthetic peptide
AVIALDGSYRYYA

SEQ ID NO: 164 - protein, 12 aa, Artificial Sequence
  Description of Artificial Sequence Synthetic peptide
CARVGPGGMDVR

SEQ ID NO: 165 - protein, 109 aa, Artificial Sequence
  Description of Artificial Sequence Synthetic polypeptide
DIQMTQSPSSLSASVGDRVTITCQASQDISNYLNWYQQKPGKAPKLLIYAASSLQSGVPSRFSGSGSGTDFTLTISSLQPEDFATYYCQQSYNSPRVYTFGQGTKVEIK

SEQ ID NO: 166 - protein, 11 aa, Artificial Sequence
  Description of Artificial Sequence Synthetic peptide
QASQDISNYLN

SEQ ID NO: 167 - protein, 7 aa, Artificial Sequence
  Description of Artificial Sequence Synthetic peptide
AASSLQS

SEQ ID NO: 168 - protein, 13 aa, Artificial Sequence
  Description of Artificial Sequence Synthetic peptide
CQQSYNSPRVYTF

SEQ ID NO: 169 - protein, 120 aa, Artificial Sequence
  Description of Artificial Sequence Synthetic polypeptide
QVQLVQSGAEVKKPGASVKVSCKASGGRFSTYALSWVRQAPGQGLEWMGIINPTDGSTTYAQKFQGRVTMTRDTSTSTVYMELSSLRSEDTAVYYCARDVYSSSWYSDYWGQGTLVTVSS

SEQ ID NO: 170 - protein, 9 aa, Artificial Sequence
  Description of Artificial Sequence Synthetic peptide
GRFSTYALS

SEQ ID NO: 171 - protein, 13 aa, Artificial Sequence
  Description of Artificial Sequence Synthetic peptide
GIINPTDGSTTYA

SEQ ID NO: 172 - protein, 15 aa, Artificial Sequence
  Description of Artificial Sequence Synthetic peptide
CARDVYSSSWYSDYW

SEQ ID NO: 173 - protein, 107 aa, Artificial Sequence
  Description of Artificial Sequence Synthetic polypeptide
DIQMTQSPSSLSASVGDRVTITCRASQSISNWLAWYQQKPGKAPKLLIYAASTLQSGVPSRFSGSGSGTDFTLTISSLQPEDFATYYCQQSYSVPFTFGPGTKVDIK

SEQ ID NO: 174 - protein, 11 aa, Artificial Sequence
  Description of Artificial Sequence Synthetic peptide
RASQSISNWLA

SEQ ID NO: 175 - protein, 7 aa, Artificial Sequence
  Description of Artificial Sequence Synthetic peptide
AASTLQS

SEQ ID NO: 176 - protein, 11 aa, Artificial Sequence [sequence omitted]

SEQ ID NO: 177 - protein, 116 aa, Artificial Sequence
  Description of Artificial Sequence Synthetic polypeptide
EVQLLESGGGLVQPGGSLRLSCAASGFPFSTYAIHWVRQAPGKGLEWVSAISGSGGSTYYADSVKGRFTISRDNSKNTLYLQMNSLRAEDTAVYYCAGPDWYFDLWGRGTLVTVSS

SEQ ID NO: 178 - protein, 9 aa, Artificial Sequence
  Description of Artificial Sequence Synthetic peptide
FPFSTYAIH

SEQ ID NO: 179 - protein, 13 aa, Artificial Sequence
  Description of Artificial Sequence Synthetic peptide
SAISGSGGSTYYA

SEQ ID NO: 180 - protein, 11 aa, Artificial Sequence
  Description of Artificial Sequence Synthetic peptide
CAGPDWYFDLW

SEQ ID NO: 181 - protein, 107 aa, Artificial Sequence
  Description of Artificial Sequence Synthetic polypeptide
DIQMTQSPSSLSASVGDRVTITCRASQAISNSLAWYQQKPGKAPKLLIYAASTLQRGVPSRFSGSGSGTDFTLTISSLQPEDFATYYCQQYYSTPYTFGQGTKLEIK

SEQ ID NO: 182 - protein, 11 aa, Artificial Sequence
  Description of Artificial Sequence Synthetic peptide
RASQAISNSLA

SEQ ID NO: 183 - protein, 7 aa, Artificial Sequence
  Description of Artificial Sequence Synthetic peptide
AASTLQR

SEQ ID NO: 184 - protein, 11 aa, Artificial Sequence [sequence omitted]

SEQ ID NO: 185 - protein, 126 aa, Artificial Sequence
  Description of Artificial Sequence Synthetic polypeptide
QVQLVQSGAEVKKPGASVKVSCKASGGTFNNYAINWVRQAPGQGLEWMGTINPRDGDTTYAQKFQGRVTMTRDTSTSTVYMELSSLRSEDTAVYYCARVGYYGSGSYYSYYGMDVWGQGTTVTVSS

SEQ ID NO: 186 - protein, 9 aa, Artificial Sequence
  Description of Artificial Sequence Synthetic peptide
GTFNNYAIN

SEQ ID NO: 187 - protein, 13 aa, Artificial Sequence
  Description of Artificial Sequence Synthetic peptide
GTINPRDGDTTYA

SEQ ID NO: 188 - protein, 21 aa, Artificial Sequence
  Description of Artificial Sequence Synthetic peptide
CARVGYYGSGSYYSYYGMDVW

SEQ ID NO: 189 - protein, 107 aa, Artificial Sequence
  Description of Artificial Sequence Synthetic polypeptide
DIQMTQSPSSLSASVGDRVTITCRASQNITNWLAWYQQKPGKAPKLLIYAASSLQSGVPSRFSGSGSGTDFTLTISSLQPEDFATYYCQQAYSFPWTFGQGTKVEIK

SEQ ID NO: 190 - protein, 11 aa, Artificial Sequence
  Description of Artificial Sequence Synthetic peptide
RASQNITNWLA

SEQ ID NO: 191 - protein, 7 aa, Artificial Sequence
  Description of Artificial Sequence Synthetic peptide
AASSLQS

SEQ ID NO: 192 - protein, 11 aa, Artificial Sequence
  Description of Artificial Sequence Synthetic peptide
CQQAYSFPWTF

SEQ ID NO: 193 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 194 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 195 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 196 - protein, 241 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 197 - protein, 228 aa, Mus musculus
MGAWAMLYGVSMLCVLDLGQPSVVEEPGCGPGKVQNGSGNNTRCCSLYAPGKEDCPKERCICVTPEYHCGDPQCKICKHYPCQPGQRVESQGDIVFGFRCVACAMGTFSAGRDGHCRLWTNCSQFGFLTMFPGNKTHNAVCIPEPLPTEQYGHLTVIFLVMAACIFFLTTVQLGLHIWQLRRQHMCPRETQPFAEVQLSAEDACSFQFPEEERGEQTEEKCHLGGRWP

SEQ ID NO: 198 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 199 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 200 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 201 - protein, 585 aa, Homo sapiens
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPR

SEQ ID NO: 202 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 203 - protein, 124 aa, Artificial Sequence
  Description of Artificial Sequence Synthetic polypeptide
QVQLVQSGAEVKKPGASVKVSCKASGGTFSSYAISWVRQAPGQGLEWMGWVSGYNGNANYAQKFQGRVTMTRDTSTSTVYMELSSLRSEDTAVYYCARDRVESGYSYHDAFDIWGQGTMVTVSS

SEQ ID NO: 204 - protein, 9 aa, Artificial Sequence
  Description of Artificial Sequence Synthetic peptide
GTFSSYAIS

SEQ ID NO: 205 - protein, 13 aa, Artificial Sequence
  Description of Artificial Sequence Synthetic peptide
GWVSGYNGNANYA

SEQ ID NO: 206 - protein, 19 aa, Artificial Sequence
  Description of Artificial Sequence Synthetic peptide
CARDRVESGYSYHDAFDIW

SEQ ID NO: 207 - protein, 107 aa, Artificial Sequence
  Description of Artificial Sequence Synthetic polypeptide
DIQMTQSPSSLSASVGDRVTITCRTSQSIRRYLNWYQQKPGKAPKLLIYAASSLQSGVPSRFSGSGSGTDFTLTISSLQPEDFATYYCQQSYSIPWTFGPGTKVDIK

SEQ ID NO: 208 - protein, 11 aa, Artificial Sequence
  Description of Artificial Sequence Synthetic peptide
RTSQSIRRYLN

SEQ ID NO: 209 - protein, 7 aa, Artificial Sequence
  Description of Artificial Sequence Synthetic peptide
AASSLQS

SEQ ID NO: 210 - protein, 11 aa, Artificial Sequence [sequence omitted]

SEQ ID NO: 211 - protein, 119 aa, Artificial Sequence
  Description of Artificial Sequence Synthetic polypeptide
QVQLVQSGAEVKKPGASVKVSCKASGYTFTSYGISWVRQAPGQGLEWMGWMNPNNGNTVYAQKFQGRVTMTRDTSTSTVYMELSSLRSEDTAVYYCAKDSDWYGAFDIWGQGTMVTVSS

SEQ ID NO: 212 - protein, 9 aa, Artificial Sequence
  Description of Artificial Sequence Synthetic peptide
YTFTSYGIS

SEQ ID NO: 213 - protein, 13 aa, Artificial Sequence
  Description of Artificial Sequence Synthetic peptide
GWMNPNNGNTVYA

SEQ ID NO: 214 - protein, 14 aa, Artificial Sequence
  Description of Artificial Sequence Synthetic peptide
CAKDSDWYGAFDIW

SEQ ID NO: 215 - protein, 107 aa, Artificial Sequence
  Description of Artificial Sequence Synthetic polypeptide
DIQMTQSPSSLSASVGDRVTITCQASQSISRWLAWYQQKPGKAPKLLIYSASSLQSGVPSRFSGSGSGTDFTLTISSLQPEDFATYYCQQSYSTPWTFGQGTKLEIK

SEQ ID NO: 216 - protein, 11 aa, Artificial Sequence
  Description of Artificial Sequence Synthetic peptide
QASQSISRWLA

SEQ ID NO: 217 - protein, 7 aa, Artificial Sequence
  Description of Artificial Sequence Synthetic peptide
SASSLQS

SEQ ID NO: 218 - protein, 11 aa, Artificial Sequence [sequence omitted]

SEQ ID NO: 219 - protein, 124 aa, Artificial Sequence
  Description of Artificial Sequence Synthetic polypeptide
QVQLVQSGAEVKKPGASVKVSCKASGYTFTSYDINWVRQAPGQGLEWIGWMNTNSGDTGYAQKFQGRVTMTRDTSTSTVYMELSSLRSEDTAVYYCARQAYSSSWYWYGWFDPWGQGTLVTVSS

SEQ ID NO: 220 - protein, 9 aa, Artificial Sequence
  Description of Artificial Sequence Synthetic peptide
YTFTSYDIN

SEQ ID NO: 221 - protein, 13 aa, Artificial Sequence
  Description of Artificial Sequence Synthetic peptide
GWMNTNSGDTGYA

SEQ ID NO: 222 - protein, 19 aa, Artificial Sequence
  Description of Artificial Sequence Synthetic peptide
CARQAYSSSWYWYGWFDPW

SEQ ID NO: 223 - protein, 107 aa, Artificial Sequence
  Description of Artificial Sequence Synthetic polypeptide
DIQMTQSPSSLSASVGDRVTITCQASHDIDNYLNWYQQKPGKAPKLLIYAASNLQSGVPSRFSGSGSGTDFTLTISSLQPEDFATYYCQQANSFPLTFGPGTKVDIK

SEQ ID NO: 224 - protein, 11 aa, Artificial Sequence
  Description of Artificial Sequence Synthetic peptide
QASHDIDNYLN

SEQ ID NO: 225 - protein, 7 aa, Artificial Sequence
  Description of Artificial Sequence Synthetic peptide
AASNLQS

SEQ ID NO: 226 - protein, 11 aa, Artificial Sequence [sequence omitted]

SEQ ID NO: 227 - protein, 118 aa, Artificial Sequence
  Description of Artificial Sequence Synthetic polypeptide
QVQLVQSGAEVKKPGASVKVSCKASGYTFPNYGITWVRQAPGQGLEWMGWMNPNSGYTGYAQNFQGRVTMTRDTSTSTVYMELSSLRSEDTAVYYCARGMPGRGFDPWGQGTLVTVSS

SEQ ID NO: 228 - protein, 9 aa, Artificial Sequence
  Description of Artificial Sequence Synthetic peptide
YTFPNYGIT

SEQ ID NO: 229 - protein, 13 aa, Artificial Sequence
  Description of Artificial Sequence Synthetic peptide
GWMNPNSGYTGYA

SEQ ID NO: 230 - protein, 13 aa, Artificial Sequence
  Description of Artificial Sequence Synthetic peptide
CARGMPGRGFDPW

SEQ ID NO: 231 - protein, 108 aa, Artificial Sequence
  Description of Artificial Sequence Synthetic polypeptide
EIVMTQSPATLSVSPGERATLSCRASQSVSSNYLAWYQQKPGQAPRLLIYGASTRATGIPARFSGSGSGTEFTLTISSLQSEDFAVYYCQQYHTYPPTFGQGTKLEIK

SEQ ID NO: 232 - protein, 12 aa, Artificial Sequence
  Description of Artificial Sequence Synthetic peptide
RASQSVSSNYLA

SEQ ID NO: 233 - protein, 7 aa, Artificial Sequence
  Description of Artificial Sequence Synthetic peptide
GASTRAT

SEQ ID NO: 234 - protein, 11 aa, Artificial Sequence [sequence omitted]

SEQ ID NO: 235 - protein, 121 aa, Artificial Sequence
  Description of Artificial Sequence Synthetic polypeptide
QVQLVQSGAEVKKPGASVKVSCKASGYTFSSDYIHWVRQAPGQGLEWMGRINPSGGSTLYARRFQGRVTMTRDTSTSTVYMELSSLRSEDTAVYYCARERGAADTWYFDLWGRGTLVTVSS

SEQ ID NO: 236 - protein, 9 aa, Artificial Sequence
  Description of Artificial Sequence Synthetic peptide
YTFSSDYIH

SEQ ID NO: 237 - protein, 13 aa, Artificial Sequence
  Description of Artificial Sequence Synthetic peptide
GRINPSGGSTLYA

SEQ ID NO: 238 - protein, 16 aa, Artificial Sequence
  Description of Artificial Sequence Synthetic peptide
CARERGAADTWYFDLW

SEQ ID NO: 239 - protein, 107 aa, Artificial Sequence
  Description of Artificial Sequence Synthetic polypeptide
DIQMTQSPSSLSASVGDRVTITCRASQSISSYLNWYQQKPGKAPKLLIYGASSLQSGVPSRFSGSGSGTDFTLTISSLQPEDFATYYCQQSYRTPLTFGGGTKVEIK

SEQ ID NO: 240 - protein, 11 aa, Artificial Sequence
  Description of Artificial Sequence Synthetic peptide
RASQSISSYLN

SEQ ID NO: 241 - protein, 7 aa, Artificial Sequence
  Description of Artificial Sequence Synthetic peptide
GASSLQS

SEQ ID NO: 242 - protein, 11 aa, Artificial Sequence [sequence omitted]

SEQ ID NO: 243 - protein, 118 aa, Artificial Sequence
  Description of Artificial Sequence Synthetic polypeptide
QVQLVQSGAEVKKPGASVKVSCKASGGTFSSYAISWVRQAPGQGLEWMGIIDPTGGATAYAQEFQGRVTMTRDTSTSTVYMELSSLRSEDTAVYYCARVGYYYGMDVWGQGTMVTVSS

SEQ ID NO: 244 - protein, 9 aa, Artificial Sequence
  Description of Artificial Sequence Synthetic peptide
GTFSSYAIS

SEQ ID NO: 245 - protein, 13 aa, Artificial Sequence
  Description of Artificial Sequence Synthetic peptide
GIIDPTGGATAYA

SEQ ID NO: 246 - protein, 13 aa, Artificial Sequence [sequence omitted]

SEQ ID NO: 247 - protein, 107 aa, Artificial Sequence
  Description of Artificial Sequence Synthetic polypeptide
DIQMTQSPSSLSASVGDRVTITCRASQSVSTYLNWYQQKPGKAPKLLIYAASSLQSGVPSRFSGSGSGTDFTLTISSLQPEDFATYYCQQSYSTPLTFGGGTKVEIK

SEQ ID NO: 248 - protein, 11 aa, Artificial Sequence
  Description of Artificial Sequence Synthetic peptide
RASQSVSTYLN

SEQ ID NO: 249 - protein, 7 aa, Artificial Sequence
  Description of Artificial Sequence Synthetic peptide
AASSLQS

SEQ ID NO: 250 - protein, 11 aa, Artificial Sequence [sequence omitted]

SEQ ID NO: 251 - protein, 120 aa, Artificial Sequence
  Description of Artificial Sequence Synthetic polypeptide
QVQLVQSGAEVKKPGSSVKVSCKASGGTFSSYAISWVRQAPGQGLEWMGGIIPISSATSIPQKFQGRVTITADESTSTAYMELSSLRSEDTAVYYCARSYDSRYYGMDVWGQGTTVTVSS

SEQ ID NO: 252 - protein, 9 aa, Artificial Sequence
  Description of Artificial Sequence Synthetic peptide
GTFSSYAIS

SEQ ID NO: 253 - protein, 13 aa, Artificial Sequence
  Description of Artificial Sequence Synthetic peptide
GGIIPISSATSIP

SEQ ID NO: 254 - protein, 15 aa, Artificial Sequence
  Description of Artificial Sequence Synthetic peptide
CARSYDSRYYGMDVW

SEQ ID NO: 255 - protein, 108 aa, Artificial Sequence
  Description of Artificial Sequence Synthetic polypeptide
EIVMTQSPATLSVSPGERATLSCRASQTVGSRYLAWYQQKPGQAPRLLIYGASTRATGIPARFSGSGSGTEFTLTISSLQSEDFAVYYCQQYYSTPWTFGQGTRLEIK

SEQ ID NO: 256 - protein, 12 aa, Artificial Sequence
  Description of Artificial Sequence Synthetic peptide
RASQTVGSRYLA

SEQ ID NO: 257 - protein, 7 aa, Artificial Sequence
  Description of Artificial Sequence Synthetic peptide
GASTRAT

SEQ ID NO: 258 - protein, 11 aa, Artificial Sequence [sequence omitted]

SEQ ID NO: 259 - protein, 116 aa, Artificial Sequence
  Description of Artificial Sequence Synthetic polypeptide
QVQLVQSGAEVKKPGASVKVSCKASGGTFSSYAISWVRQAPGQGLEWMGWMNPNSGDTGYAQKFQGRVTMTRDTSTSTVYMELSSLRSEDTAVYYCARGPWYFDLWGRGTLVTVSS

SEQ ID NO: 260 - protein, 9 aa, Artificial Sequence
  Description of Artificial Sequence Synthetic peptide
GTFSSYAIS

SEQ ID NO: 261 - protein, 13 aa, Artificial Sequence
  Description of Artificial Sequence Synthetic peptide
GWMNPNSGDTGYA

SEQ ID NO: 262 - protein, 11 aa, Artificial Sequence
  Description of Artificial Sequence Synthetic peptide
CARGPWYFDLW

SEQ ID NO: 263 - protein, 107 aa, Artificial Sequence
  Description of Artificial Sequence Synthetic polypeptide
DIQMTQSPSSLSASVGDRVTITCRVSQGISNSLAWYQQKPGKAPKLLIYAASSLQSGVPSRFSGSGSGTDFTLTISSLQPEDFATYYCQQSYSTVYTFGQGTKLEIK

SEQ ID NO: 264 - protein, 11 aa, Artificial Sequence
  Description of Artificial Sequence Synthetic peptide
RVSQGISNSLA

SEQ ID NO: 265 - protein, 7 aa, Artificial Sequence
  Description of Artificial Sequence Synthetic peptide
AASSLQS

SEQ ID NO: 266 - protein, 11 aa, Artificial Sequence [sequence omitted]

SEQ ID NO: 267 - protein, 116 aa, Artificial Sequence
  Description of Artificial Sequence Synthetic polypeptide
EVQLLESGGGLVKPGGSLRLSCAASGFMFSSYSMNWVRQAPGKGLEWVSYISGNSGYTNYADSVKGRFTISRDDSKNTLYLQMNSLKTEDTAVYYCARRLHGMDVWGQGTTVTVSS

SEQ ID NO: 268 - protein, 9 aa, Artificial Sequence
  Description of Artificial Sequence Synthetic peptide
FMFSSYSMN

SEQ ID NO: 269 - protein, 13 aa, Artificial Sequence
  Description of Artificial Sequence Synthetic peptide
SYISGNSGYTNYA

SEQ ID NO: 270 - protein, 11 aa, Artificial Sequence
  Description of Artificial Sequence Synthetic peptide
CARRLHGMDVW

SEQ ID NO: 271 - protein, 107 aa, Artificial Sequence
  Description of Artificial Sequence Synthetic polypeptide
DIQMTQSPSSLSASVGDRVTITCRASQSISTWLAWYQQKPGKAPKLLIYDASNLETGVPSRFSGSGSGTDFTLTISSLQPEDFATYYCQQAYRFPVAFGGGTKVEIK

SEQ ID NO: 272 - protein, 11 aa, Artificial Sequence
  Description of Artificial Sequence Synthetic peptide
RASQSISTWLA

SEQ ID NO: 273 - protein, 7 aa, Artificial Sequence
  Description of Artificial Sequence Synthetic peptide
DASNLET

SEQ ID NO: 274 - protein, 11 aa, Artificial Sequence
  Description of Artificial Sequence Synthetic peptide
CQQAYRFPVAF

SEQ ID NO: 275 - protein, 118 aa, Artificial Sequence
  Description of Artificial Sequence Synthetic polypeptide
EVQLLESGGGLVQPGGSLRLSCAASGFTFSSYWMHWVRQAPGKGLEWVSVISNSGGATYYADSVKGRFTISRDNSKNTLYLQMNSLRAEDTAVYYCAREGWGYGMDVWGQGTTVTVSS

SEQ ID NO: 276 - protein, 9 aa, Artificial Sequence
  Description of Artificial Sequence Synthetic peptide
FTFSSYWMH

SEQ ID NO: 277 - protein, 13 aa, Artificial Sequence
  Description of Artificial Sequence Synthetic peptide
SVISNSGGATYYA

SEQ ID NO: 278 - protein, 13 aa, Artificial Sequence
  Description of Artificial Sequence Synthetic peptide
CAREGWGYGMDVW

SEQ ID NO: 279 - protein, 107 aa, Artificial Sequence
  Description of Artificial Sequence Synthetic polypeptide
DIQMTQSPSSLSASVGDRVTITCRASQDIRNYLAWYQQKPGKAPKLLIYTASTLQSGVPSRFSGSGSGTDFTLTISSLQPEDFATYYCQQTFRTPLTFGGGTKVEIK

SEQ ID NO: 280 - protein, 11 aa, Artificial Sequence
  Description of Artificial Sequence Synthetic peptide
RASQDIRNYLA

SEQ ID NO: 281 - protein, 7 aa, Artificial Sequence
  Description of Artificial Sequence Synthetic peptide
TASTLQS

SEQ ID NO: 282 - protein, 11 aa, Artificial Sequence
  Description of Artificial Sequence Synthetic peptide
CQQTFRTPLTF

SEQ ID NO: 283 - protein, 122 aa, Artificial Sequence
  Description of Artificial Sequence Synthetic polypeptide
QVQLVQSGAEVKKPGASVKVSCKASGYTFTNYYMHWVRQAPGQGLEWMGLITPSGGRTKYAQKFQGRVTMTRDTSTSTVYMELSSLRSEDTAVYYCAREMEYSSSWYWFDPWGQGTLVTVSS

SEQ ID NO: 284 - protein, 9 aa, Artificial Sequence
  Description of Artificial Sequence Synthetic peptide
YTFTNYYMH

SEQ ID NO: 285 - protein, 13 aa, Artificial Sequence
  Description of Artificial Sequence Synthetic peptide
GLITPSGGRTKYA

SEQ ID NO: 286 - protein, 17 aa, Artificial Sequence
  Description of Artificial Sequence Synthetic peptide
CAREMEYSSSWYWFDPW

SEQ ID NO: 287 - protein, 107 aa, Artificial Sequence
  Description of Artificial Sequence Synthetic polypeptide
DIQMTQSPSSLSASVGDRVTITCQASQGISSYLNWYQQKPGKAPKLLIYAASTLQSGVPSRFSGSGSGTDFTLTISSLQPEDFATYYCQQSYSTPLTFGGGTKVEIK

SEQ ID NO: 288 - protein, 11 aa, Artificial Sequence
  Description of Artificial Sequence Synthetic peptide
QASQGISSYLN

SEQ ID NO: 289 - protein, 7 aa, Artificial Sequence
  Description of Artificial Sequence Synthetic peptide
AASTLQS

SEQ ID NO: 290 - protein, 11 aa, Artificial Sequence [sequence omitted]

SEQ ID NO: 291 - protein, 123 aa, Artificial Sequence
  Description of Artificial Sequence Synthetic polypeptide
QVQLVQSGAEVKKPGASVKVSCKASGYTFTRNYIHWVRQAPGQGLEWMGWINPKSGGTKYAQKFQGRVTMTRDTSTSTVYMELSSLRSEDTAVYYCARESGVVATEYWYFDLWGRGTLVTVSS

SEQ ID NO: 292 - protein, 9 aa, Artificial Sequence
  Description of Artificial Sequence Synthetic peptide
YTFTRNYIH

SEQ ID NO: 293 - protein, 13 aa, Artificial Sequence
  Description of Artificial Sequence Synthetic peptide
GWINPKSGGTKYA

SEQ ID NO: 294 - protein, 18 aa, Artificial Sequence
  Description of Artificial Sequence Synthetic peptide
CARESGVVATEYWYFDLW

SEQ ID NO: 295 - protein, 107 aa, Artificial Sequence [sequence omitted]

SEQ ID NO: 296 - protein, 11 aa, Artificial Sequence
  Description of Artificial Sequence Synthetic peptide
RASQSISSYLN

SEQ ID NO: 297 - protein, 7 aa, Artificial Sequence
  Description of Artificial Sequence Synthetic peptide
AASSLQS

SEQ ID NO: 298 - protein, 11 aa, Artificial Sequence [sequence omitted]

SEQ ID NO: 299 - protein, 127 aa, Artificial Sequence
  Description of Artificial Sequence Synthetic polypeptide
QVQLVQSGAEVKKPGASVKVSCKASGYTFTSYYMHWVRQAPGQGLEWMGWINPNSGGTNYAQKFQGRVTMTRDTSTSTVYMELSSLRSEDTAVYYCASPGDYCSGGSCYSDDAFDIWGQGTMVTVSS

SEQ ID NO: 300 - protein, 9 aa, Artificial Sequence
  Description of Artificial Sequence Synthetic peptide
YTFTSYYMH

SEQ ID NO: 301 - protein, 13 aa, Artificial Sequence
  Description of Artificial Sequence Synthetic peptide
GWINPNSGGTNYA

SEQ ID NO: 302 - protein, 22 aa, Artificial Sequence
  Description of Artificial Sequence Synthetic peptide
CASPGDYCSGGSCYSDDAFDIW

SEQ ID NO: 303 - protein, 113 aa, Artificial Sequence
  Description of Artificial Sequence Synthetic polypeptide
DIVMTQSPDSLAVSLGERATINCKSSQSIFYSSNSKNYLAWYQQKPGQPPKLLIYWASTRESGVPDRFSGSGSGTDFTLTISSLQAEDVAVYYCQQYYSTPLTFGPGTKVDIK

SEQ ID NO: 304 - protein, 17 aa, Artificial Sequence
  Description of Artificial Sequence Synthetic peptide
KSSQSIFYSSNSKNYLA

SEQ ID NO: 305 - protein, 7 aa, Artificial Sequence
  Description of Artificial Sequence Synthetic peptide
WASTRES

SEQ ID NO: 306 - protein, 11 aa, Artificial Sequence [sequence omitted]

SEQ ID NO: 307 - protein, 122 aa, Artificial Sequence
  Description of Artificial Sequence Synthetic polypeptide
QVQLVQSGAEVKKPGASVKVSCKASGGTFRNYAINWVRQAPGQGLEWMGWMNPNSGNTGYAQKFQGRVTMTRDTSTSTVYMELSSLRSEDTAVYYCAREDVDTASQAYFDYWGQGTLVTVSS

SEQ ID NO: 308 - protein, 9 aa, Artificial Sequence
  Description of Artificial Sequence Synthetic peptide
GTFRNYAIN

SEQ ID NO: 309 - protein, 13 aa, Artificial Sequence
  Description of Artificial Sequence Synthetic peptide
GWMNPNSGNTGYA

SEQ ID NO: 310 - protein, 17 aa, Artificial Sequence
  Description of Artificial Sequence Synthetic peptide
CAREDVDTASQAYFDYW

SEQ ID NO: 311 - protein, 107 aa, Artificial Sequence
  Description of Artificial Sequence Synthetic polypeptide
DIQMTQSPSSLSASVGDRVTITCRASQGISNYLAWYQQKPGKAPKLLIYSASNLQSGVPSRFSGSGSGTDFTLTISSLQPEDFATYYCQQGYSSRYTFGQGTKLEIK

SEQ ID NO: 312 - protein, 11 aa, Artificial Sequence
  Description of Artificial Sequence Synthetic peptide
RASQGISNYLA

SEQ ID NO: 313 - protein, 7 aa, Artificial Sequence
  Description of Artificial Sequence Synthetic peptide
SASNLQS

SEQ ID NO: 314 - protein, 11 aa, Artificial Sequence
  Description of Artificial Sequence Synthetic peptide
CQQGYSSRYTF

SEQ ID NO: 315 - protein, 120 aa, Artificial Sequence
  Description of Artificial Sequence Synthetic polypeptide
EVQLLESGGGLVKPGGSLRLSCAASGFTFSSYWMSWVRQAPGKGLEWVSSISWSSTYIYYADSVKGRFTISRDDSKNTLYLQMNSLKTEDTAVYYCARDGQLGHWHFDLWGRGTLVTVSS

SEQ ID NO: 316 - protein, 9 aa, Artificial Sequence
  Description of Artificial Sequence Synthetic peptide
FTFSSYWMS

SEQ ID NO: 317 - protein, 13 aa, Artificial Sequence
  Description of Artificial Sequence Synthetic peptide
SSISWSSTYIYYA

SEQ ID NO: 318 - protein, 15 aa, Artificial Sequence
  Description of Artificial Sequence Synthetic peptide
CARDGQLGHWHFDLW

SEQ ID NO: 319 - protein, 107 aa, Artificial Sequence
  Description of Artificial Sequence Synthetic polypeptide
DIQMTQSPSSLSASVGDRVTITCRASQSISDWLAWYQQKPGKAPKLLIYEASKLATGVPSRFSGSGSGTDFTLTISSLQPEDFATYYCQQSYTTPLTFGGGTKVEIK

SEQ ID NO: 320 - protein, 11 aa, Artificial Sequence
  Description of Artificial Sequence Synthetic peptide
RASQSISDWLA

SEQ ID NO: 321 - protein, 7 aa, Artificial Sequence
  Description of Artificial Sequence Synthetic peptide
EASKLAT

SEQ ID NO: 322 - protein, 11 aa, Artificial Sequence [sequence omitted]

SEQ ID NO: 323 - protein, 117 aa, Artificial Sequence
  Description of Artificial Sequence Synthetic polypeptide
EVQLLESGGGLVQPGGSLRLSCAASGFTFRNHWMSWVRQAPGKGLEWVSGISWNSGSIDYADSVKGRFTISRDNSKNTLYLQMNSLRAEDTAVYYCAREEYATFDYWGQGTLVTVSS

SEQ ID NO: 324 - protein, 9 aa, Artificial Sequence
  Description of Artificial Sequence Synthetic peptide
FTFRNHWMS

SEQ ID NO: 325 - protein, 13 aa, Artificial Sequence
  Description of Artificial Sequence Synthetic peptide
SGISWNSGSIDYA

SEQ ID NO: 326 - protein, 12 aa, Artificial Sequence
  Description of Artificial Sequence Synthetic peptide
CAREEYATFDYW

SEQ ID NO: 327 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPPTFGPGTKVDIK

SEQ ID NO: 328 - protein, 16 aa, Artificial Sequence
  Description of Artificial Sequence Synthetic peptide
RSSQSLLHSNGYNYLD

SEQ ID NO: 329 - protein, 7 aa, Artificial Sequence
  Description of Artificial Sequence Synthetic peptide
LGSNRAS

SEQ ID NO: 330 - protein, 11 aa, Artificial Sequence [sequence omitted]

SEQ ID NO: 331 - protein, 122 aa, Artificial Sequence
  Description of Artificial Sequence Synthetic polypeptide
QVQLVQSGAEVKKPGASVKVSCKASGGSFSDYAVSWVRQAPGQGLEWMGWMNPNSGNTGYAQKFQGRVTMTRDTSTSTVYMELSSLRSEDTAVYYCARELVRDGYNFALDYWGQGTLVTVSS

SEQ ID NO: 332 - protein, 9 aa, Artificial Sequence
  Description of Artificial Sequence Synthetic peptide
GSFSDYAVS

SEQ ID NO: 333 - protein, 13 aa, Artificial Sequence
  Description of Artificial Sequence Synthetic peptide
GWMNPNSGNTGYA

SEQ ID NO: 334 - protein, 17 aa, Artificial Sequence
  Description of Artificial Sequence Synthetic peptide
CARELVRDGYNFALDYW

SEQ ID NO: 335 - protein, 107 aa, Artificial Sequence
  Description of Artificial Sequence Synthetic polypeptide
DIQMTQSPSSLSASVGDRVTITCRASQDIRNDLAWYQQKPGKAPKLLIYAASSLQSGVPSRFSGSGSGTDFTLTISSLQPEDFATYYCQQSFSTFYTFGQGTKLEIK

SEQ ID NO: 336 - protein, 11 aa, Artificial Sequence
  Description of Artificial Sequence Synthetic peptide
RASQDIRNDLA

SEQ ID NO: 337 - protein, 7 aa, Artificial Sequence
  Description of Artificial Sequence Synthetic peptide
AASSLQS

SEQ ID NO: 338 - protein, 11 aa, Artificial Sequence
  Description of Artificial Sequence Synthetic peptide
CQQSFSTFYTF

SEQ ID NO: 339 - protein, 117 aa, Artificial Sequence
  Description of Artificial Sequence Synthetic polypeptide
QVQLVQSGAEVKKPGASVKVSCKASGYIFTNYWIQWVRQAPGQGLEWMGWINPHSGATKYAERFQGRVTMTRDTSTSTVYMELSSLRSEDTAVYYCARGLGSAFDIWGQGTMVTVSS

SEQ ID NO: 340 - protein, 9 aa, Artificial Sequence
  Description of Artificial Sequence Synthetic peptide
YIFTNYWIQ

SEQ ID NO: 341 - protein, 13 aa, Artificial Sequence
  Description of Artificial Sequence Synthetic peptide
GWINPHSGATKYA

SEQ ID NO: 342 - protein, 12 aa, Artificial Sequence
  Description of Artificial Sequence Synthetic peptide
CARGLGSAFDIW

SEQ ID NO: 343 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 344 - protein, 16 aa, Artificial Sequence
  Description of Artificial Sequence Synthetic peptide
RSSQSLLHSNGYNYLD

SEQ ID NO: 345 - protein, 7 aa, Artificial Sequence
  Description of Artificial Sequence Synthetic peptide
LGSNRAS

SEQ ID NO: 346 - protein, 11 aa, Artificial Sequence [sequence omitted]

SEQ ID NO: 347 - protein, 126 aa, Artificial Sequence
  Description of Artificial Sequence Synthetic polypeptide
QVQLVQSGAEVKKPGASVKVSCKASGYTFTSHDIDWVRQAPGQGLEWMGWMNPNNGNTVYAQRFQGRVTMTRDTSTSTVYMELSSLRSEDTAVYYCARWKVYSGSYYGGAGYFDLWGRGTLVTVSS

SEQ ID NO: 348 - protein, 9 aa, Artificial Sequence
  Description of Artificial Sequence Synthetic peptide
YTFTSHDID

SEQ ID NO: 349 - protein, 13 aa, Artificial Sequence
  Description of Artificial Sequence Synthetic peptide
GWMNPNNGNTVYA

SEQ ID NO: 350 - protein, 21 aa, Artificial Sequence
  Description of Artificial Sequence Synthetic peptide
CARWKVYSGSYYGGAGYFDLW

SEQ ID NO: 351 - protein, 107 aa, Artificial Sequence
  Description of Artificial Sequence Synthetic polypeptide
DIQMTQSPSSLSASVGDRVTITCRASQSISSYLNWYQQKPGKAPKLLIYRASHLEGGVPSRFSGSGSGTDFTLTISSLQPEDFATYYCQQADSLPLTFGQGTKVEIK

SEQ ID NO: 352 - protein, 11 aa, Artificial Sequence
  Description of Artificial Sequence Synthetic peptide
RASQSISSYLN

SEQ ID NO: 353 - protein, 7 aa, Artificial Sequence
  Description of Artificial Sequence Synthetic peptide
RASHLEG

SEQ ID NO: 354 - protein, 11 aa, Artificial Sequence [sequence omitted]

SEQ ID NO: 355 - protein, 120 aa, Artificial Sequence
  Description of Artificial Sequence Synthetic polypeptide
QVQLVQSGAEVKKPGASVKVSCKASGYTFTSHDIDWVRQAPGQGLEWMGWINPSGDSTSYAQKFQGRVTMTRDTSTSTVYMELSSLRSEDTAVYYCARYYGGNSYAFDIWGQGTMVTVSS

SEQ ID NO: 356 - protein, 9 aa, Artificial Sequence
  Description of Artificial Sequence Synthetic peptide
YTFTSHDID

SEQ ID NO: 357 - protein, 13 aa, Artificial Sequence
  Description of Artificial Sequence Synthetic peptide
GWINPSGDSTSYA

SEQ ID NO: 358 - protein, 15 aa, Artificial Sequence
  Description of Artificial Sequence Synthetic peptide
CARYYGGNSYAFDIW

SEQ ID NO: 359 - protein, 107 aa, Artificial Sequence
  Description of Artificial Sequence Synthetic polypeptide
DIQMTQSPSSLSASVGDRVTITCRASESISPWVAWYQQKPGKAPKLLIYAASTLQSGVPSRFSGSGSGTDFTLTISSLQPEDFATYYCQQGTSTPYTFGQGTKLEIK

SEQ ID NO: 360 - protein, 11 aa, Artificial Sequence
  Description of Artificial Sequence Synthetic peptide
RASESISPWVA

SEQ ID NO: 361 - protein, 7 aa, Artificial Sequence
  Description of Artificial Sequence Synthetic peptide
AASTLQS

SEQ ID NO: 362 - protein, 11 aa, Artificial Sequence
  Description of Artificial Sequence Synthetic peptide
CQQGTSTPYTF

SEQ ID NO: 363 - protein, 117 aa, Artificial Sequence
  Description of Artificial Sequence Synthetic polypeptide
QVQLVQSGAEVKKPGASVKVSCKASGYTFTSYGISWVRQAPGQGLEWMGWMNPNSGNTGYAQKFQGRVTMTRDTSTSTVYMELSSLRSEDTAVYYCAREVYGDLDYWGQGTLVTVSS

SEQ ID NO: 364 - protein, 9 aa, Artificial Sequence
  Description of Artificial Sequence Synthetic peptide
YTFTSYGIS

SEQ ID NO: 365 - protein, 13 aa, Artificial Sequence
  Description of Artificial Sequence Synthetic peptide
GWMNPNSGNTGYA

SEQ ID NO: 366 - protein, 12 aa, Artificial Sequence
  Description of Artificial Sequence Synthetic peptide
CAREVYGDLDYW

SEQ ID NO: 367 - protein, 107 aa, Artificial Sequence
  Description of Artificial Sequence Synthetic polypeptide
DIQMTQSPSSLSASVGDRVTITCRASQGIDNWLAWYQQKPGKAPKLLIYGASTLQSGVPSRFSGSGSGTDFTLTISSLQPEDFATYYCQQSYTARFTFGPGTKVDIK

SEQ ID NO: 368 - protein, 11 aa, Artificial Sequence
  Description of Artificial Sequence Synthetic peptide
RASQGIDNWLA

SEQ ID NO: 369 - protein, 7 aa, Artificial Sequence
  Description of Artificial Sequence Synthetic peptide
GASTLQS

SEQ ID NO: 370 - protein, 11 aa, Artificial Sequence
  Description of Artificial Sequence Synthetic peptide
CQQSYTARFTF

SEQ ID NO: 371 - protein, 121 aa, Artificial Sequence
  Description of Artificial Sequence Synthetic polypeptide
QVQLVQSGAEVKKPGASVKVSCKASGGSFSNYAINWVRQAPGQGLEWMGWMNPYSGNTGYAQKFQGRVTMTRDTSTSTVYMELSSLRSEDTAVYYCAREITANYYYGMDVWGQGTKVTVSS

SEQ ID NO: 372 - protein, 9 aa, Artificial Sequence
  Description of Artificial Sequence Synthetic peptide
GSFSNYAIN

SEQ ID NO: 373 - protein, 13 aa, Artificial Sequence
  Description of Artificial Sequence Synthetic peptide
GWMNPYSGNTGYA

SEQ ID NO: 374 - protein, 16 aa, Artificial Sequence
  Description of Artificial Sequence Synthetic peptide
CAREITANYYYGMDVW

SEQ ID NO: 375 - protein, 107 aa, Artificial Sequence
  Description of Artificial Sequence Synthetic polypeptide
DIQMTQSPSSLSASVGDRVTITCQASQDISNYLNWYQQKPGKAPKLLIYEASVLESGVPSRFSGSGSGTDFTLTISSLQPEDFATYYCQQSYTTQYTFGQGTKVEIK

SEQ ID NO: 376 - protein, 11 aa, Artificial Sequence
  Description of Artificial Sequence Synthetic peptide
QASQDISNYLN

SEQ ID NO: 377 - protein, 7 aa, Artificial Sequence
  Description of Artificial Sequence Synthetic peptide
EASVLES

SEQ ID NO: 378 - protein, 11 aa, Artificial Sequence [sequence omitted]

SEQ ID NO: 379 - protein, 119 aa, Artificial Sequence
  Description of Artificial Sequence Synthetic polypeptide
QVQLVQSGAEVKKPGSSVKVSCKASGGTFSTYAINWVRQAPGQGLEWMGIINPTDGDTSYAQKFQGRVTITADESTSTAYMELSSLRSEDTAVYYCARAAYYYYGMDVWGQGTTVTVSS

SEQ ID NO: 380 - protein, 9 aa, Artificial Sequence
  Description of Artificial Sequence Synthetic peptide
GTFSTYAIN

SEQ ID NO: 381 - protein, 13 aa, Artificial Sequence
  Description of Artificial Sequence Synthetic peptide
GIINPTDGDTSYA

SEQ ID NO: 382 - protein, 14 aa, Artificial Sequence
  Description of Artificial Sequence Synthetic peptide
CARAAYYYYGMDVW

SEQ ID NO: 383 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALHIPYTFGQGTKLEIK

SEQ ID NO: 384 - protein, 16 aa, Artificial Sequence
  Description of Artificial Sequence Synthetic peptide
RSSQSLLHSNGYNYLD

SEQ ID NO: 385 - protein, 7 aa, Artificial Sequence
  Description of Artificial Sequence Synthetic peptide
LGSNRAS

SEQ ID NO: 386 - protein, 11 aa, Artificial Sequence [sequence omitted]

SEQ ID NO: 387 - protein, 125 aa, Artificial Sequence
  Description of Artificial Sequence Synthetic polypeptide
QVQLVQSGAEVKKPGASVKVSCKASGYSFTSHDIDWVRQAPGQGLEWMGWINPNSGGTNYAQKFQGRVTMTRDTSTSTVYMELSSLRSEDTAVYYCARIRGYYGSGSYHDAFDIWGQGTTVTVSS

SEQ ID NO: 388 - protein, 9 aa, Artificial Sequence
  Description of Artificial Sequence Synthetic peptide
YSFTSHDID

SEQ ID NO: 389 - protein, 13 aa, Artificial Sequence
  Description of Artificial Sequence Synthetic peptide
GWINPNSGGTNYA

SEQ ID NO: 390 - protein, 20 aa, Artificial Sequence
  Description of Artificial Sequence Synthetic peptide
CARIRGYYGSGSYHDAFDIW

SEQ ID NO: 391 - protein, 107 aa, Artificial Sequence
  Description of Artificial Sequence Synthetic polypeptide
DIQMTQSPSSLSASVGDRVTITCRASQTISTYLNWYQQKPGKAPKLLIYAASSLQSGVPSRFSGSGSGTDFTLTISSLQPEDFATYYCQQSYSTPWTFGQGTKVEIK

SEQ ID NO: 392 - protein, 11 aa, Artificial Sequence
  Description of Artificial Sequence Synthetic peptide
RASQTISTYLN

SEQ ID NO: 393 - protein, 7 aa, Artificial Sequence
  Description of Artificial Sequence Synthetic peptide
AASSLQS

SEQ ID NO: 394 - protein, 11 aa, Artificial Sequence [sequence omitted]

SEQ ID NO: 395 - protein, 235 aa, Macaca fascicularis
MCACGTLCCLALLCAASLGQRPTGGPGCGPGRLLLGTGKDARCCRVHPTRCCRDYQSEECCSEWDCVCVQPEFHCGNPCCTTCQHHPCPSGQGVQPQGKFSFGFRCVDCALGTFSRGHDGHCKPWTDCTQFGFLTVFPGNKTHNAVCVPGSPPAEPPGWLTIVLLAVAACVLLLTSAQLGLHIWQLGSQPTGPRETQLLLEVPPSTEDASSCQFPEEERGERLAEEKGRLGDLWV

(19) United States
(12) Patent Application Publication
(10) Pub. No.: US 2022/0106398 A1
(43) Pub. Date: Apr. 7, 2022

(54) ANTI-GITR ANTIGEN-BINDING DOMAINS AND USES THEREOF

(71) Applicant: IGM Biosciences, Inc., Mountain View, CA (US)

(72) Inventors: Ramesh BALIGA, Redwood City, CA (US); Bruce Alan KEYT, Hillsborough, CA (US)

(21) Appl. No.: 17/427,882

(22) PCT Filed: Feb. 6, 2020
(86) PCT No.: PCT/US2020/017083

§ 371 Date: Aug. 2, 2021

Related U.S. Application Data
(60) Provisional application No. 62/836,810, filed on Apr. 22, 2019.
(60) Provisional application No. 62/802,775, filed on Feb. 8, 2019.

(51) Int. Cl. C07K 16/28

(52) U.S. Cl. CPC C07K 16/2878; C07K 2317/21; C07K 2317/565; C07K 2317/622; C07K 2317/92; C07K 2317/33

(57) ABSTRACT

This disclosure provides an antibody or antigen-binding fragment or derivative thereof that specifically binds to glu-cocorticoid-induced TNF receptor (GITR). Also provided are polynucleotides encoding the antibody or antigen-binding fragment or derivative thereof and vectors and host cell comprising said polynucleotides. This disclosure further provides methods for producing and/or using an antibody or antigen-binding fragment or derivative thereof that specifically binds to glucocorticoid-induced TNF re-ceptor (GITR).

### CROSS-REFERENCE TO RELATED APPLICATIONS

This application claims the benefit of U.S. Provisional Patent Application Ser. No. 62/802,775, filed Feb. 8, 2019 and U.S. Provisional Patent Application Ser. No. 62/836,810, filed Apr. 22, 2019, which are each incorporated herein by reference in their entireties.

### SEQUENCE LISTING

The instant application contains a Sequence Listing which has been submitted electronically in ASCII format and is hereby incorporated by reference in its entirety. The ASCII copy was created on Sep. 30, 2019, is named 09789-025WO1-Sequence-Listing, and is 212,148 bytes in size.

### BACKGROUND

Tumor Necrosis Factor superfamily receptor (TNFSFR) proteins are important targets for immuno oncology therapeutic agents. For example, agonist monoclonal antibodies directed against TNFSFR targets such as CD40, GITR, CD137, and OX40, among many others, are currently in clinical trials for myriad cancer indications.

In many instances, activation of the TNFSFR targets requires that at least three non-interacting receptor monomers on the surface of a cell expressing the receptor be cross-linked to form a stabilized receptor trimer, resulting in signal transduction across the cell membrane. Clustering of TNFSFR protein trimers into “rafts” of trimers leads to more effective activation of the signaling cascade. (See, Valley et al., J. Biol. Chem., 287(25):21265-21278, 2012). Typically, clustering of TNFSFR on the surface of a cell can be accomplished via engagement by multimeric, e.g., trimeric ligands. Recent work has demonstrated that a multimeric agonistic IgM antibody directed against the TNFSFR DRS can effectively bind multiple DRS receptor monomers on the surface of a cell in the absence of secondary cross linking, and with increased cytotoxicity over an IgG molecule with identical antigen-binding domains. See U.S. Pat. No. 9,938,347, which is incorporated herein by reference in its entirety.

Glucocorticoid-Induced TNF Receptor (“GITR,” also known as AITR or TNFRSF18) is a TNFSFR expressed on activated T cells, NK cells, and NKT cells. GITR has low basal expression on nave murine effector CD4+ and CD8 T+ cells, and very low expression on human effector T cells, e.g., cytotoxic T lymphocytes (CTLs). Murine and human CD4+ CD25+ FoxP3+ regulatory T cells (Tregs) constitutively express GITR (Schaer, D A, et al., Curr Opin. Immunol. 24:217-224 (2012)). Upon activation, both effector T cells and Tregs upregulate GITR expression (Id.). Interaction with its trimeric ligand (GITRL, TNFSF18, AITRL) expressed on activated antigen-presenting cells (APCs), e.g., macrophages and dendritic cells (DC), provides enhanced costimulatory proliferation and effector functions in CD4+ and CD8 effector T cells (Tone M, et al., Proc Natl Acad Sci USA. 100:15059-15064 (2003); Ronchetti, S., et al., Eur J. Immunol. 34:613-622 (2004)). GITR signaling can also block the immunosuppressive abilities of Tregs, thereby enhancing cytotoxic T lymphocyte (CTL) function (Shimizu, J., et al., Nature Immunol 3:135-142 (2002)). GITR agonist mAbs can enhance the effector functions and proliferation of CTLs and can impair intratumoral CD25+ CD4+ FoxP3+ Treg stability (Schaer D A, et al. Cancer Immunol Res. 1:320-31 (2013)). Agonist monoclonal antibodies directed against GITR have shown therapeutic activity in preclinical models (See, e.g., Cohen, A D, et al., PLoS One 5(5):e10436. doi: 10.1371/journal.pone.0010436(2010)) . Moreover, several GITR IgG agonist mAbs are being investigated in human clinical trials, including, but not limited to TRX518 (humanized agly IgG1) (Schaer, D A, et al., Curr Opin. Immunol. 24:217-224 (2012)); MK-4166 (ClinicalTrials.gov #NCT02132754); and INCAGN1876 (ClinicalTrials.gov #NCT02697591). Typical bivalent IgG agonist antibodies, however, require cross-linking to sufficiently engage TNFSFRs on the surface of a cell to trigger signal transduction.

There remains a need to develop more potent and therefore more effective GITR agonist antibodies for use in cancer immunotherapy.

### SUMMARY

The disclosure provides an antibody or antigen-binding fragment or derivative thereof including an antigen-binding domain that specifically binds to glucocorticoid-induced TNF receptor (GITR), where the antigen-binding domain includes a heavy chain variable region (VH) and light chain variable region (VL), where the VH and VL include six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 include, respectively, the amino acid sequences SEQ ID NO: 2, SEQ ID NO: 3, SEQ ID NO:4, SEQ ID NO: 6, SEQ ID NO: 7, and SEQ ID NO: 8, SEQ ID NO: 10, SEQ ID NO: 11, SEQ ID NO: 12, SEQ ID NO: 14, SEQ ID NO: 15, and SEQ ID NO: 16; SEQ ID NO: 18, SEQ ID NO: 19, SEQ ID NO: 20, SEQ ID NO: 22, SEQ ID NO: 23, and SEQ ID NO: 24, SEQ ID NO: 26, SEQ ID NO: 27, SEQ ID NO: 28, SEQ ID NO: 30, SEQ ID NO: 31, and SEQ ID NO: 32, SEQ ID NO: 34, SEQ ID NO: 35, SEQ ID NO: 36, SEQ ID NO: 38, SEQ ID NO: 39, and SEQ ID NO: 40; SEQ ID NO: 42, SEQ ID NO: 43, SEQ ID NO: 44, SEQ ID NO: 46, SEQ ID NO: 47, and SEQ ID NO: 48; SEQ ID NO: 50, SEQ ID NO: 51, SEQ ID NO: 52, SEQ ID NO: 54, SEQ ID NO: 55, and SEQ ID NO: 56; SEQ ID NO: 58, SEQ ID NO: 59, SEQ ID NO: 60, SEQ ID NO: 62, SEQ ID NO: 63, and SEQ ID NO: 64; SEQ ID NO: 66, SEQ ID NO: 74, SEQ ID NO: 75, SEQ ID NO: 76, SEQ ID NO: 78, SEQ ID NO: 79, and SEQ ID NO: 80; SEQ ID NO: 82, SEQ ID NO: 83, SEQ ID NO: 84, SEQ ID NO: 86, SEQ ID NO: 87, and SEQ ID NO: 88; SEQ ID NO: 90, SEQ ID NO: 91, SEQ ID NO: 92, SEQ ID NO: 94, SEQ ID NO: 95, and SEQ ID NO: 96; SEQ ID NO: 98, SEQ ID NO: 99, SEQ ID NO: 100, SEQ ID NO: 102, SEQ ID NO: 103, and SEQ ID NO: 104; SEQ ID NO: 106, SEQ ID NO: 107, SEQ ID NO: 108, SEQ ID NO: 110, SEQ ID NO: 111, and SEQ ID NO: 112; SEQ ID NO: 114, SEQ ID NO: 115, SEQ ID NO: 116, SEQ ID NO: 118, SEQ ID NO: 119, and SEQ ID NO: 120; SEQ ID NO: 122, SEQ ID NO: 123, SEQ ID NO: 124, SEQ ID NO: 126, SEQ ID NO: 127, and SEQ ID NO: 128; SEQ ID NO: 130, SEQ ID NO: 131, SEQ ID NO: 132, SEQ ID NO: 134, SEQ ID NO: 135, and SEQ ID NO: 136; SEQ ID NO: 138, SEQ ID NO: 139, SEQ ID NO: 140, SEQ ID NO: 142, SEQ ID NO: 143, and SEQ ID NO: 144; SEQ ID NO: 146, SEQ ID NO: 147, SEQ ID NO: 148, SEQ ID NO: 150, SEQ ID NO: 151, and SEQ ID NO: 152; SEQ ID NO: 154, SEQ ID NO: 155, SEQ ID NO: 156, SEQ ID NO: 158, SEQ ID NO: 159, and SEQ ID NO: 160; SEQ ID NO: 162, SEQ ID NO: 163, SEQ ID NO: 164, SEQ ID NO: 166, SEQ ID NO: 167, and SEQ ID NO: 168; SEQ ID NO: 170, SEQ ID NO: 171, SEQ ID NO: 172, SEQ ID NO: 174, SEQ ID NO: 175, and SEQ ID NO: 176; SEQ ID NO: 178, SEQ ID NO: 179, SEQ ID NO: 180, SEQ ID NO: 182, SEQ ID NO: 183, and SEQ ID NO: 184; SEQ ID NO: 186, SEQ ID NO: 187, SEQ ID NO: 188, SEQ ID NO: 190, SEQ ID NO: 191, and SEQ ID NO: 192; SEQ ID NO: 204, SEQ ID NO: 205, SEQ ID NO: 206, SEQ ID NO: 208, SEQ ID NO: 209, and SEQ ID NO: 210; SEQ ID NO: 212, SEQ ID NO: 213, SEQ ID NO: 214, SEQ ID NO: 216, SEQ ID NO: 217, and SEQ ID NO: 218; SEQ ID NO: 220, SEQ ID NO: 221, SEQ ID NO: 222, SEQ ID NO: 224, SEQ ID NO: 225, and SEQ ID NO: 226; SEQ ID NO: 228, SEQ ID NO: 229, SEQ ID NO: 230, SEQ ID NO: 232, SEQ ID NO: 233, and SEQ ID NO: 234; SEQ ID NO: 236, SEQ ID NO: 237, SEQ ID NO: 238, SEQ ID NO: 240, SEQ ID NO: 241, and SEQ ID NO: 242; SEQ ID NO: 244, SEQ ID NO: 245, SEQ ID NO: 246, SEQ ID NO: 248, SEQ ID NO: 249, and SEQ ID NO: 250; SEQ ID NO: 252, SEQ ID NO: 253, SEQ ID NO: 254, SEQ ID NO: 256, SEQ ID NO: 257, and SEQ ID NO: 258; SEQ ID NO: 260, SEQ ID NO: 261, SEQ ID NO: 262, SEQ ID NO: 264, SEQ ID NO: 265, and SEQ ID NO: 266; SEQ ID NO: 268, SEQ ID NO: 269, SEQ ID NO: 270, SEQ ID NO: 272, SEQ ID NO: 273, and SEQ ID NO: 274; SEQ ID NO: 276, SEQ ID NO: 277, SEQ ID NO: 278, SEQ ID NO: 280, SEQ ID NO: 281, and SEQ ID NO: 282; SEQ ID NO: 284, SEQ ID NO: 285, SEQ ID NO: 286, SEQ ID NO: 288, SEQ ID NO: 289, and SEQ ID NO: 290; SEQ ID NO: 292, SEQ ID NO: 293, SEQ ID NO: 294, SEQ ID NO: 296, SEQ ID NO: 297, and SEQ ID NO: 298; SEQ ID NO: 300, SEQ ID NO: 301, SEQ ID NO: 302, SEQ ID NO: 304, SEQ ID NO: 305, and SEQ ID NO: 306; SEQ ID NO: 308, SEQ ID NO: 309, SEQ ID NO: 310, SEQ ID NO: 312, SEQ ID NO: 313, and SEQ ID NO: 314; SEQ ID NO: 316, SEQ ID NO: 317, SEQ ID NO: 318, SEQ ID NO: 320, SEQ ID NO: 321, and SEQ ID NO: 322; SEQ ID NO: 324, SEQ ID NO: 325, SEQ ID NO: 326, SEQ ID NO: 328, SEQ ID NO: 329, and SEQ ID NO: 330; SEQ ID NO: 332, SEQ ID NO: 333, SEQ ID NO: 334, SEQ ID NO: 336, SEQ ID NO: 337, and SEQ ID NO: 338; SEQ ID NO: 340, SEQ ID NO: 341, SEQ ID NO: 342, SEQ ID NO: 344, SEQ ID NO: 345, and SEQ ID NO: 346; SEQ ID NO: 348, SEQ ID NO: 349, SEQ ID NO: 350, SEQ ID NO: 352, SEQ ID NO: 353, and SEQ ID NO: 354; SEQ ID NO: 356, SEQ ID NO: 357, SEQ ID NO: 358, SEQ ID NO: 360, SEQ ID NO: 361, and SEQ ID NO: 362; SEQ ID NO: 364, SEQ ID NO: 365, SEQ ID NO: 366, SEQ ID NO: 368, SEQ ID NO: 369, and SEQ ID NO: 370; SEQ ID NO: 372, SEQ ID NO: 373, SEQ ID NO: 374, SEQ ID NO: 376, SEQ ID NO: 377, and SEQ ID NO: 378; SEQ ID NO: 380, SEQ ID NO: 381, SEQ ID NO: 382, SEQ ID NO: 384, SEQ ID NO: 385, and SEQ ID NO: 386; or SEQ ID NO: 388, SEQ ID NO: 389, SEQ ID NO: 390, SEQ ID NO: 392, SEQ ID NO: 393, and SEQ ID NO: 394; or where the HCDR1, HCDR2, HCDR3, LCDR1, LCDR2, and LCDR3 include the amino acid sequences recited above except for one or two amino acid substitutions in one or more of the CDRs.

In certain aspects, the VH of the antibody or fragment or derivative thereof can further include framework regions (HFWs) HFW1, HFW2, HFW3, and HFW4, and the VL can further include framework regions (LFWs) LFW1, LFW2, LFW3, and LFW4. In certain aspects the framework regions can be derived from a human antibody. Alternatively, the framework regions can be derived from a non-human antibody.

In certain aspects the VH of the antibody or fragment or derivative thereof includes the amino acid sequence SEQ ID NO: 1, SEQ ID NO: 9, SEQ ID NO: 17, SEQ ID NO: 25, SEQ ID NO: 33, SEQ ID NO: 41, SEQ ID NO: 49, SEQ ID NO: 57, SEQ ID NO: 65, SEQ ID NO: 73, SEQ ID NO: 81, SEQ ID NO: 89, SEQ ID NO: 97, SEQ ID NO: 105, SEQ ID NO: 113, SEQ ID NO: 121, SEQ ID NO: 129, SEQ ID NO: 137, SEQ ID NO: 145, SEQ ID NO: 153, SEQ ID NO: 161, SEQ ID NO: 169, SEQ ID NO: 177, SEQ ID NO: 185, SEQ ID NO: 203, SEQ ID NO: 211, SEQ ID NO: 219, SEQ ID NO: 227, SEQ ID NO: 235, SEQ ID NO: 243, SEQ ID NO: 251, SEQ ID NO: 259, SEQ ID NO: 267, SEQ ID NO: 275, SEQ ID NO: 283, SEQ ID NO: 291, SEQ ID NO: 299, SEQ ID NO: 307, SEQ ID NO: 315, SEQ ID NO: 323, SEQ ID NO: 331, SEQ ID NO: 339, SEQ ID NO: 347, SEQ ID NO: 355, SEQ ID NO: 363, SEQ ID NO: 371, SEQ ID NO: 379, or SEQ ID NO: 387. In certain aspects the VL of the antibody or fragment or derivative thereof includes the amino acid sequence SEQ ID NO: 5, SEQ ID NO: 13, SEQ ID NO: 21, SEQ ID NO: 29, SEQ ID NO: 37, SEQ ID NO: 45, SEQ ID NO: 53, SEQ ID NO: 61, SEQ ID NO: 69, SEQ ID NO: 77, SEQ ID NO: 85, SEQ ID NO: 93, SEQ ID NO: 101, SEQ ID NO: 109, SEQ ID NO: 117, SEQ ID NO: 125, SEQ ID NO: 133, SEQ ID NO: 141, SEQ ID NO: 149, SEQ ID NO: 157, SEQ ID NO: 165, SEQ ID NO: 173, SEQ ID NO: 181, SEQ ID NO: 189, SEQ ID NO: 207, SEQ ID NO: 215, SEQ ID NO: 223, SEQ ID NO: 231, SEQ ID NO: 239, SEQ ID NO: 247, SEQ ID NO: 255, SEQ ID NO: 263, SEQ ID NO: 271, SEQ ID NO: 279, SEQ ID NO: 287, SEQ ID NO: 295, SEQ ID NO: 303, SEQ ID NO: 311, SEQ ID NO: 319, SEQ ID NO: 327, SEQ ID NO: 335, SEQ ID NO: 343, SEQ ID NO: 351, SEQ ID NO: 359, SEQ ID NO: 367, SEQ ID NO: 375, SEQ ID NO: 383, or SEQ ID NO: 391. In certain aspects the VH and VL of the antibody or fragment or derivative thereof include, respectively, the amino acid sequences SEQ ID NO: 1 and SEQ ID NO: 5, SEQ ID NO: 9 and SEQ ID NO: 13, SEQ ID NO: 17 and SEQ ID NO: 21, SEQ ID NO: 25 and SEQ ID NO: 29, SEQ ID NO: 33 and SEQ ID NO: 37, SEQ ID NO: 41 and SEQ ID NO: 45, SEQ ID NO: 49 and SEQ ID NO: 53, SEQ ID NO: 57 and SEQ ID NO: 61, SEQ ID NO: 65 and SEQ ID NO: 69, SEQ ID NO: 73 and SEQ ID NO: 77, SEQ ID NO: 81 and SEQ ID NO: 85, SEQ ID NO: 89 and SEQ ID NO: 93, SEQ ID NO: 97 and SEQ ID NO: 101, SEQ ID NO: 105 and SEQ ID NO: 109, SEQ ID NO: 113 and SEQ ID NO: 117, SEQ ID NO: 121 and SEQ ID NO: 125, SEQ ID NO: 129 and SEQ ID NO: 133, SEQ ID NO: 137 and SEQ ID NO: 141, SEQ ID NO: 145 and SEQ ID NO: 149, SEQ ID NO: 153 and SEQ ID NO: 157, SEQ ID NO: 161 and SEQ ID NO: 165, SEQ ID NO: 169 and SEQ ID NO: 173, SEQ ID NO: 177 and SEQ ID NO: 181, SEQ ID NO: 185 and SEQ ID NO: 189, SEQ ID NO: 203 and SEQ ID NO: 207, SEQ ID NO: 211 and SEQ ID NO: 215, SEQ ID NO: 219 and SEQ ID NO: 223, SEQ ID NO: 227 and SEQ ID NO: 231, SEQ ID NO: 235 and SEQ ID NO: 239, SEQ ID NO: 243 and SEQ ID NO: 247, SEQ ID NO: 251 and SEQ ID NO: 255, SEQ ID NO: 259 and SEQ ID NO: 263, SEQ ID NO: 267 and SEQ ID NO: 271, SEQ ID NO: 275 and SEQ ID NO: 279, SEQ ID NO: 283 and SEQ ID NO: 287, SEQ ID NO: 291 and SEQ ID NO: 295, SEQ ID NO: 299 and SEQ ID NO: 303, SEQ ID NO: 307 and SEQ ID NO: 311, SEQ ID NO: 315 and SEQ ID NO: 319, SEQ ID NO: 323 and SEQ ID NO: 327, SEQ ID NO: 331 and SEQ ID NO: 335, SEQ ID NO: 339 and SEQ ID NO: 343, SEQ ID NO: 347 and SEQ ID NO: 351, SEQ ID NO: 355 and SEQ ID NO: 359, SEQ ID NO: 363 and SEQ ID NO: 367, SEQ ID NO: 371 and SEQ ID NO: 375, SEQ ID NO: 379 and SEQ ID NO: 383, or SEQ ID NO: 387 and SEQ ID NO: 391.

The disclosure further provides an antibody or antigen-binding fragment or derivative thereof including an antigen-binding domain that specifically binds to glucocorticoid-induced TNF receptor (GITR), where the antigen-binding domain includes a heavy chain variable region (VH) and light chain variable region (VL), where the VH includes an amino acid sequence at least 80%, 85%, 90%, 95%, or 100% identical to SEQ ID NO: 1, SEQ ID NO: 9, SEQ ID NO: 17, SEQ ID NO: 25, SEQ ID NO: 33, SEQ ID NO: 41, SEQ ID NO: 49, SEQ ID NO: 57, SEQ ID NO: 65, SEQ ID NO: 73, SEQ ID NO: 81, SEQ ID NO: 89, SEQ ID NO: 97, SEQ ID NO: 105, SEQ ID NO: 113, SEQ ID NO: 121, SEQ ID NO: 129, SEQ ID NO: 137, SEQ ID NO: 145, SEQ ID NO: 153, SEQ ID NO: 161, SEQ ID NO: 169, SEQ ID NO: 177, SEQ ID NO: 185, SEQ ID NO: 203, SEQ ID NO: 211, SEQ ID NO: 219, SEQ ID NO: 227, SEQ ID NO: 235, SEQ ID NO: 243, SEQ ID NO: 251, SEQ ID NO: 259, SEQ ID NO: 267, SEQ ID NO: 275, SEQ ID NO: 283, SEQ ID NO: 291, SEQ ID NO: 299, SEQ ID NO: 307, SEQ ID NO: 315, SEQ ID NO: 323, SEQ ID NO: 331, SEQ ID NO: 339, SEQ ID NO: 347, SEQ ID NO: 355, SEQ ID NO: 363, SEQ ID NO: 371, SEQ ID NO: 379, or SEQ ID NO: 387. In certain aspects the VL includes an amino acid sequence at least 80%, 85%, 90%, 95%, or 100% identical to SEQ ID NO: 5, SEQ ID NO: 13, SEQ ID NO: 21, SEQ ID NO: 29, SEQ ID NO: 37, SEQ ID NO: 45, SEQ ID NO: 53, SEQ ID NO: 61, SEQ ID NO: 69, SEQ ID NO: 77, SEQ ID NO: 85, SEQ ID NO: 93, SEQ ID NO: 101, SEQ ID NO: 109, SEQ ID NO: 117, SEQ ID NO: 125, SEQ ID NO: 133, SEQ ID NO: 141, SEQ ID NO: 149, SEQ ID NO: 157, SEQ ID NO: 165, SEQ ID NO: 173, SEQ ID NO: 181, SEQ ID NO: 189, SEQ ID NO: 207, SEQ ID NO: 215, SEQ ID NO: 223, SEQ ID NO: 231, SEQ ID NO: 239, SEQ ID NO: 247, SEQ ID NO: 255, SEQ ID NO: 263, SEQ ID NO: 271, SEQ ID NO: 279, SEQ ID NO: 287, SEQ ID NO: 295, SEQ ID NO: 303, SEQ ID NO: 311, SEQ ID NO: 319, SEQ ID NO: 327, SEQ ID NO: 335, SEQ ID NO: 343, SEQ ID NO: 351, SEQ ID NO: 359, SEQ ID NO: 367, SEQ ID NO: 375, SEQ ID NO: 383, or SEQ ID NO: 391.

The disclosure further provides an antibody or antigen-binding fragment or derivative thereof including an antigen-binding domain that specifically binds to glucocorticoid-induced TNF receptor (GITR), where the antigen-binding domain includes a heavy chain variable region (VH) and light chain variable region (VL), where the VL includes an amino acid sequence at least 80%, 85%, 90%, 95%, or 100% identical to SEQ ID NO: 5, SEQ ID NO: 13, SEQ ID NO: 21, SEQ ID NO: 29, SEQ ID NO: 37, SEQ ID NO: 45, SEQ ID NO: 53, SEQ ID NO: 61, SEQ ID NO: 69, SEQ ID NO: 77, SEQ ID NO: 85, SEQ ID NO: 93, SEQ ID NO: 101, SEQ ID NO: 109, SEQ ID NO: 117, SEQ ID NO: 125, SEQ ID NO: 133, SEQ ID NO: 141, SEQ ID NO: 149, SEQ ID NO: 157, SEQ ID NO: 165, SEQ ID NO: 173, SEQ ID NO: 181, SEQ ID NO: 189, SEQ ID NO: 207, SEQ ID NO: 215, SEQ ID NO: 223, SEQ ID NO: 231, SEQ ID NO: 239, SEQ ID NO: 247, SEQ ID NO: 255, SEQ ID NO: 263, SEQ ID NO: 271, SEQ ID NO: 279, SEQ ID NO: 287, SEQ ID NO: 295, SEQ ID NO: 303, SEQ ID NO: 311, SEQ ID NO: 319, SEQ ID NO: 327, SEQ ID NO: 335, SEQ ID NO: 343, SEQ ID NO: 351, SEQ ID NO: 359, SEQ ID NO: 367, SEQ ID NO: 375, SEQ ID NO: 383, or SEQ ID NO: 391. in certain aspects the VH includes an amino acid sequence at least 80%, 85%, 90%, 95%, or 100% identical to SEQ ID NO: 1, SEQ ID NO: 9, SEQ ID NO: 17, SEQ ID NO: 25, SEQ ID NO: 33, SEQ ID NO: 41, SEQ ID NO: 49, SEQ ID NO: 57, SEQ ID NO: 65, SEQ ID NO: 73, SEQ ID NO: 81, SEQ ID NO: 89, SEQ ID NO: 97, SEQ ID NO: 105, SEQ ID NO: 113, SEQ ID NO: 121, SEQ ID NO: 129, SEQ ID NO: 137, SEQ ID NO: 145, SEQ ID NO: 153, SEQ ID NO: 161, SEQ ID NO: 169, SEQ ID NO: 177, SEQ ID NO: 185, SEQ ID NO: 203, SEQ ID NO: 211, SEQ ID NO: 219, SEQ ID NO: 227, SEQ ID NO: 235, SEQ ID NO: 243, SEQ ID NO: 251, SEQ ID NO: 259, SEQ ID NO: 267, SEQ ID NO: 275, SEQ ID NO: 283, SEQ ID NO: 291, SEQ ID NO: 299, SEQ ID NO: 307, SEQ ID NO: 315, SEQ ID NO: 323, SEQ ID NO: 331, SEQ ID NO: 339, SEQ ID NO: 347, SEQ ID NO: 355, SEQ ID NO: 363, SEQ ID NO: 371, SEQ ID NO: 379, or SEQ ID NO: 387.

The disclosure further provides an antibody or antigen-binding fragment or derivative thereof including an antigen-binding domain that specifically binds to glucocorticoid-induced TNF receptor (GITR), where the antigen-binding domain includes a heavy chain variable region (VH) and light chain variable region (VL), where the VH and VL include amino acid sequences at least 80%, at least 85%, at least 90%, at least 95% or 100% identical to the mature VH and VL amino acid sequences including, respectively, SEQ ID NO: 1 and SEQ ID NO: 5, SEQ ID NO: 9 and SEQ ID NO: 13, SEQ ID NO: 17 and SEQ ID NO: 21, SEQ ID NO: 25 and SEQ ID NO: 29, SEQ ID NO: 33 and SEQ ID NO: 37, SEQ ID NO: 41 and SEQ ID NO: 45, SEQ ID NO: 49 and SEQ ID NO: 53, SEQ ID NO: 57 and SEQ ID NO: 61, SEQ ID NO: 65 and SEQ ID NO: 69, SEQ ID NO: 73 and SEQ ID NO: 77, SEQ ID NO: 81 and SEQ ID NO: 85, SEQ ID NO: 89 and SEQ ID NO: 93, SEQ ID NO: 97 and SEQ ID NO: 101, SEQ ID NO: 105 and SEQ ID NO: 109, SEQ ID NO: 113 and SEQ ID NO: 117, SEQ ID NO: 121 and SEQ ID NO: 125, SEQ ID NO: 129 and SEQ ID NO: 133, SEQ ID NO: 137 and SEQ ID NO: 141, SEQ ID NO: 145 and SEQ ID NO: 149, SEQ ID NO: 153 and SEQ ID NO: 157, SEQ ID NO: 161 and SEQ ID NO: 165, SEQ ID NO: 169 and SEQ ID NO: 173, SEQ ID NO: 177 and SEQ ID NO: 181, SEQ ID NO: 185 and SEQ ID NO: 189, SEQ ID NO: 203 and SEQ ID NO: 207, SEQ ID NO: 211 and SEQ ID NO: 215, SEQ ID NO: 219 and SEQ ID NO: 223, SEQ ID NO: 227 and SEQ ID NO: 231, SEQ ID NO: 235 and SEQ ID NO: 239, SEQ ID NO: 243 and SEQ ID NO: 247, SEQ ID NO: 251 and SEQ ID NO: 255, SEQ ID NO: 259 and SEQ ID NO: 263, SEQ ID NO: 267 and SEQ ID NO: 271, SEQ ID NO: 275 and SEQ ID NO: 279, SEQ ID NO: 283 and SEQ ID NO: 287, SEQ ID NO: 291 and SEQ ID NO: 295, SEQ ID NO: 299 and SEQ ID NO: 303, SEQ ID NO: 307 and SEQ ID NO: 311, SEQ ID NO: 315 and SEQ ID NO: 319, SEQ ID NO: 323 and SEQ ID NO: 327, SEQ ID NO: 331 and SEQ ID NO: 335, SEQ ID NO: 339 and SEQ ID NO: 343, SEQ ID NO: 347 and SEQ ID NO: 351, SEQ ID NO: 355 and SEQ ID NO: 359, SEQ ID NO: 363 and SEQ ID NO: 367, SEQ ID NO: 371 and SEQ ID NO: 375, SEQ ID NO: 379 and SEQ ID NO: 383, or SEQ ID NO: 387 and SEQ ID NO: 391.

An antibody or fragment or derivative thereof provided by this disclosure can be, e.g., a complete antibody, an Fv fragment, a single-chain Fv fragment (scFv), or a disulfide-linked Fv fragment (sdFv).

In certain aspects, an antibody or fragment or derivative thereof provided by this disclosure can be a single bivalent binding unit that includes two antigen-binding domains where at least one antigen-binding domain specifically binds to GITR. In certain aspects the binding unit includes two heavy chains each including a heavy chain constant region or fragment or variant thereof. In certain aspects at least one heavy chain constant region or variant thereof of the binding unit is fused to a copy of the VH. In certain aspects, both heavy chain constant regions or fragments or variants thereof of the single binding unit are fused to a copy of the VH. In certain aspects the heavy chain constant regions are IgG heavy chain constant regions or fragments or variants thereof. In certain aspects the single binding unit further includes two light chains each including a light chain constant region or fragment or variant thereof, and where at least one light chain constant region is fused to a copy of the VL. In certain aspects, both light chain constant regions or fragments or variants thereof of the single binding unit are fused to a copy of the VL. In certain aspects, the single binding unit can be, e.g., a complete antibody, an Fab fragment, an Fab′ fragment, or an F(ab′)2 fragment. In certain aspects, the single binding unit is a human antibody, fragment, or derivative thereof.

In certain aspects, an antibody or fragment or derivative thereof provided by this disclosure can be a multimeric antibody including two, five, or six bivalent binding units and four, ten, or twelve antigen-binding domains, where at least one, two, three, four, five, six, seven, eight, nine, ten, eleven, or twelve antigen-binding domains specifically binds to GITR. Each binding unit includes two heavy chains each including an IgA or IgM constant region or a multimerizing fragment or variant thereof, and at least one heavy chain constant region of each binding unit is fused to a copy of the VH. In certain aspects a multimeric antibody or fragment or derivative thereof as provided by this disclosure can be a human antibody, fragment, or derivative thereof.

In those aspects where the antibody or fragment or derivative thereof is dimeric, it can include two bivalent IgA binding units and a J chain or fragment or variant thereof, where each binding unit includes two IgA heavy chain constant regions or multimerizing fragments or variants thereof. The dimeric antibody or fragment or derivative thereof can further include a secretory component, or fragment or variant thereof. In certain aspects, the IgA heavy chain constant regions or fragments or variants thereof each include a Cα3-tp domain, and can further include a Cα1 domain, a Cα2 domain, an IgA hinge region, or any combination thereof.

In those aspects where the antibody or fragment or derivative thereof is hexameric or pentameric, it can include five or six bivalent IgM binding units, where each binding unit includes two IgM heavy chain constant regions or multimerizing fragments or variants thereof. In certain aspects, the IgM heavy chain constant regions or fragments or variants thereof each include a Cμ4-tp domain or fragment or variant thereof, and can further include a Cμ1 domain, a Cμ2 domain, a Cμ3 domain, or any combination thereof. In those aspects where the antibody or fragment or derivative thereof is pentameric, it can further include a J chain, or fragment thereof, or variant thereof.

In certain aspects, each binding unit of a multimeric antibody or fragment or derivative thereof as provided by this disclosure can further include two light chains each including a light chain constant region or fragment or variant thereof. In certain aspects, at least one, two, three, four, five, six, seven eight, nine, ten, eleven, or twelve light chain constant regions are fused to a copy of the VL.

In certain aspects, an antibody or fragment or derivative thereof as provided by this disclosure can be multispecific.

In certain aspects, an antibody or fragment or derivative thereof as provided by this disclosure can specifically bind to human GITR, mouse GITR, and/or non-human primate GITR, e.g., cynomolgus monkey GITR.

In certain aspects, an antibody or fragment or derivative thereof as provided by the disclosure can specifically bind to GITR with an affinity characterized by a dissociation constant KD no greater than 500 nM, 100 nM, 50.0 nM, 40.0 nM, 30.0 nM, 20.0 nM, 10.0 nM, 9.0 nM, 8.0 nM, 7.0 nM, 6.0 nM, 5.0 nM, 4.0 nM, 3.0 nM, 2.0 nM, 1.0 nM, 0.50 nM, 0.10 nM, 0.050 nM, 0.01 nM, 0.005 nM, or 0.001 nM; and where the GITR is human GITR, mouse GITR, cynomolgus monkey GITR, or any combination thereof.

The disclosure further provides a composition that includes the provided antibody or fragment or derivative thereof.

Also provided by the disclosure is a polynucleotide that includes a nucleic acid sequence that encodes the antibody or fragment or derivative thereof of any one of claims 1 to 35 or any subunit thereof. Also provided is a vector and/or a host cell that includes the provided polynucleotide. Also provided is a method of producing the provided antibody or fragment or derivative thereof, where the method includes culturing the provided host cell and recovering the antibody or fragment or derivative thereof.

The disclosure further provides a method of inducing GITR-mediated activation in a GITR-expressing cell, where the method includes contacting the GITR-expressing cell with the provided antibody or fragment or derivative thereof.

The disclosure further provides a method of inducing GITR translocation and clustering in GITR-expressing T cells, where the method includes contacting GITR-expressing T cells with the provided antibody or fragment or derivative thereof.

The disclosure further provides a method of treating cancer, where the method includes administering to a subject in need of treatment an effective amount of the provided antibody or fragment or derivative thereof, where the antibody or fragment or derivative thereof can activate GITR-expressing CTL cells thereby triggering a tumoricidal CTL response. In certain aspects the subject to be treated is human.

### BRIEF DESCRIPTION OF THE DRAWINGS/FIGURES

FIG. 1A-D: Binding of IgMJ* and IgG versions of GITR-Mab-3, GITR-Mab-6, GITR-Mab-11, and GITR-Mab 14 to human GITR-expressing HEK 293 cells. FIG. 1A: GITR-Mab-3, FIG. 1B: GITR-Mab-6, FIG. 1C: GITR-Mab-11, FIG. 1D: GITR-Mab-14. Closed circles: IgG, open squares: IgMJ*.

FIG. 2A-B: Activity of IgMJ* and IgG versions of GITR-Mab-11 and GITR-Mab-14 in a reporter assay using NFKB-luc2/GITR Jurkat cells.

### DETAILED DESCRIPTION

### Definitions

It is to be noted that the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations. As used herein, the term glycoprotein refers to a protein coupled to at least one carbohydrate moiety that is attached to the protein via an oxygen-containing or a nitrogen-containing side chain of an amino acid, e.g., a serine or an asparagine.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. In certain aspects, variants can be non-naturally occurring. Non-naturally occurring variants can be produced using art-known mutagenesis techniques. Variant polypeptides can comprise conservative or non-conservative amino acid substitutions, deletions or additions. Derivatives are polypeptides, e.g., antibodies as provided herein, that have been altered so as to exhibit additional features not found on the original polypeptide. Examples include fusion proteins. Variant polypeptides can also be referred to herein as “polypeptide analogs.” As used herein a “derivative” of a polypeptide, e.g., an antibody derivative as provided herein, can also refer to a subject polypeptide having one or more amino acids chemically derivatized by reaction of a functional side group. Also included as “derivatives” are those polypeptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the present disclosure do not abrogate the binding of the polypeptide or antibody containing the amino acid sequence, to the antigen to which the binding molecule binds. Methods of identifying nucleotide and amino acid conservative substitutions which do not eliminate antigen-binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:.412-417 (1997)).

The term “polynucleotide” is intended to encompass a singular nucleic acid as well as plural nucleic acids and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, the term “a non-naturally occurring polynucleotide” or any grammatical variants thereof, is a conditional definition that explicitly excludes, but only excludes, those forms of the nucleic acid or polynucleotide that are, or might be, determined or interpreted by a judge, or an administrative or judicial body, to be “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit B-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse B-glucuronidase.

As used herein, the term “binding molecule” refers in its broadest sense to a molecule that specifically binds to a receptor, e.g., an epitope or an antigenic determinant. As described further herein, a binding molecule can comprise one of more “antigen-binding domains” described herein. A non-limiting example of a binding molecule is an antibody or fragment thereof that retains antigen-specific binding.

As used herein, the terms “binding domain” or “antigen-binding domain” refer to a region of a binding molecule that is necessary and sufficient to specifically bind to an epitope. For example, an “Fv,” e.g., a variable heavy chain and variable light chain of an antibody, either as two separate polypeptide subunits or as a single chain, is considered to be a “binding domain.” Other antigen-binding domains include, without limitation, the variable heavy chain (VHH) of an antibody derived from a camelid species, or six immunoglobulin complementarity determining regions (CDRs) expressed in a fibronectin scaffold. A “binding molecule” as described herein can include one, two, three, four, five, six, seven, eight, nine, ten, eleven, twelve or more “antigen-binding domains.”

The terms “antibody” and “immunoglobulin” can be used interchangeably herein. An antibody (or a fragment, variant, or derivative thereof as disclosed herein) includes at least the variable domain of a heavy chain (for camelid species) or at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains, an IgA antibody that includes four complete heavy chains and four complete light chains and optionally includes a J-chain and/or a secretory component, or an IgM antibody that includes ten or twelve complete heavy chains and ten or twelve complete light chains and optionally includes a J-chain or functional fragment or variant thereof.

The term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 or α1-α2)). It is the nature of this chain that determines the “isotype” of the antibody as IgG, IgM, IgA IgD, or IgE, respectively. The immunoglobulin subclasses (subtypes) e.g., IgG1, IgG2, IgG3, IgG4, IgA1, IgA2, etc. are well characterized and are known to confer functional specialization. Modified versions of each of these immunoglobulins are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are expressed, e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure, or a “binding unit.”

The term “binding unit” is used herein to refer to the portion of a binding molecule, e.g., an antibody or antigen-binding fragment thereof, which corresponds to a standard “H2L2” immunoglobulin structure, i.e., two heavy chains or fragments thereof and optionally (in the case of, e.g., camelid antibodies) two light chains or fragments thereof. In certain aspects, e.g., where the binding molecule is a bivalent IgG antibody or antigen-binding fragment thereof, the terms “binding molecule” and “binding unit” are equivalent. In other aspects, e.g., where the binding molecule is multimeric, e.g., an IgA dimer, an IgM pentamer, or an IgM hexamer, the binding molecule comprises two or more “binding units.” Two in the case of an IgA dimer, or five or six in the case of an IgM pentamer or hexamer, respectively. A binding unit need not include full-length antibody heavy and light chains, but will typically be bivalent, i.e., will include two “antigen-binding domains,” as defined above. As used herein, certain binding molecules provided in this disclosure are “dimeric,” and include two bivalent binding units that include IgA constant regions or fragments thereof. Certain binding molecules provided in this disclosure are “pentameric” or “hexameric,” and include five or six bivalent binding units that include IgM constant regions or fragments thereof. A binding molecule comprising two or more, e.g., two, five, or six binding units, is referred to herein as “multimeric.”

As used herein, an “IgM-like antibody” refers to a variant antibody that still retains the ability to form hexamers, or in association with J-chain, form pentamers. An IgM-like antibody typically includes at least the Cμ4-tp domain of the IgM constant region but can include heavy chain constant region domains from other antibody isotypes, e.g., IgG, from the same species or from a different species. An IgM-like antibody can likewise be an antibody fragment in which one or more constant regions are deleted, as long as the IgM-like antibody is capable of forming hexamers and/or pentamers. Thus, an IgM-like antibody can be a hybrid IgM/IgG antibody or can be a “multimerizing fragment” of an IgM antibody. As used herein, a “multimeric antibody” refers to an antibody comprising two or more binding units.

The terms “valency,” “bivalent,” “multivalent” and grammatical equivalents, refer to the number of antigen-binding domains in given binding molecule, antibody, or binding unit. As such, the terms “bivalent”, “tetravalent”, and “hexavalent” in reference to a given binding molecule, e.g., an IgM antibody, denote the presence of two antigen-binding domains, four antigen-binding domains, and six antigen-binding domains, respectively. In a typical IgM-derived binding molecule where each binding unit is bivalent, the binding molecule itself can have 10 or 12 valencies. A bivalent or multivalent binding molecule can be monospecific, i.e., all of the antigen-binding domains are the same, or can be bispecific or multispecific, e.g., where two or more antigen-binding domains are different, e.g., bind to different epitopes on the same antigen, or bind to entirely different antigens.

The term “epitope” includes any molecular determinant capable of specific binding to an antibody. In certain aspects, an epitope can include chemically active surface groupings of molecules such as amino acids, sugar side chains, phosphoryl, or sulfonyl, and, in certain aspects, can have a three-dimensional structural characteristics, and or specific charge characteristics. An epitope is a region of a target that is bound by an antibody.

The term “target” is used in the broadest sense to include substances that can be bound by a binding molecule. A target can be, e.g., a polypeptide, a nucleic acid, a carbohydrate, a lipid, or other molecule. Moreover, a “target” can, for example, be a cell, an organ, or an organism that comprises an epitope bound that can be bound by a binding molecule.

Both the light and heavy chains are divided into regions of structural and functional homology. The terms “constant” and “variable” are used functionally. In this regard, it will be appreciated that the variable domains of both the variable light (VL) and variable heavy (VH) chain portions determine antigen recognition and specificity. Conversely, the constant domains of the light chain (CL) and the heavy chain (e.g., CH1, CH2 or CH3) confer biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 (or CH4 in the case of IgM) and CL domains actually comprise the carboxy-terminus of the heavy and light chain, respectively.

A “full length IgM antibody heavy chain” is a polypeptide that includes, in N-terminal to C-terminal direction, an antibody heavy chain variable domain (VH), an antibody constant heavy chain constant domain 1 (CM1 or Cμ1), an antibody heavy chain constant domain 2 (CM2 or Cμ2), an antibody heavy chain constant domain 3 (CM3 or CO), and an antibody heavy chain constant domain 4 (CM4 or Cμ4) that can include a tailpiece.

As indicated above, variable region(s) allows a binding molecule to selectively recognize and specifically bind epitopes on antigens. That is, the VL domain and VH domain, or subset of the complementarity determining regions (CDRs), of a binding molecule, e.g., an antibody, combine to form the antigen-binding domain. More specifically, an antigen-binding domain can be defined by three CDRs on each of the VH and VL chains. Certain antibodies form larger structures. For example, IgA can form a molecule that includes two H2L2 binding units and a J-chain covalently connected via disulfide bonds, which can be further associated with a secretory component, and IgM can form a pentameric or hexameric molecule that includes five or six H2L2 binding units and optionally a J-chain covalently connected via disulfide bonds.

The six “complementarity determining regions” or “CDRs” present in an antibody antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domain, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

Binding molecules, e.g., antibodies or antigen-binding fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019.

By “specifically binds,” it is generally meant that a binding molecule, e.g., an antibody or fragment, variant, or derivative thereof binds to an epitope via its antigen-binding domain, and that the binding entails some complementarity between the antigen-binding domain and the epitope. According to this definition, a binding molecule is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen with an off rate (k(off)) of less than or equal to 5×10−2 sec−1, 10−2 sec−1, 5×10−3 sec−1, 10−3 sec−1, 5×10−4 sec−1, 10−4 sec−1, 5×10−5 sec−1, or 10−5 sec−1 5×10−6 sec−1, 10−6 sec−1, 5×10−7 sec−1 or 10−7 sec−1.

A binding molecule, e.g., an antibody or antigen-binding fragment, variant, or derivative disclosed herein can be said to bind a target antigen with an on rate (k(on)) of greater than or equal to 103 M−1 sec−1, 5×103 M−1 sec−1, 104 M−1 sec−1, 5×104 M−1 sec−1, 105 M−1 sec−1, 5×105 M−1 sec−1, 106 M−1 sec−1, or 5×106 M−1 sec−1 or 107 M−1 sec−1.

A binding molecule, e.g., an antibody or fragment, variant, or derivative thereof is said to competitively inhibit binding of a reference antibody or antigen binding fragment to a given epitope if it preferentially binds to that epitope to the extent that it blocks, to some degree, binding of the reference antibody or antigen binding fragment to the epitope. Competitive inhibition can be determined by any method known in the art, for example, competition ELISA assays. A binding molecule can be said to competitively inhibit binding of the reference antibody or antigen binding fragment to a given epitope by at least 90%, at least 80%, at least 70%, at least 60%, or at least 50%.

As used herein, the term “affinity” refers to a measure of the strength of the binding of an individual epitope with one or more antigen-binding domains, e.g., of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of antigen-binding domains and an antigen. See, e.g., Harlow at pages 29-34. Avidity is related to both the affinity of individual antigen-binding domains in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a between a bivalent monoclonal antibody with a receptor present at a high density on a cell surface would also be of high avidity.

Binding molecules, e.g., antibodies or fragments, variants, or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross-reactive epitope generally contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or KD no greater than 5×10−2 M, 10−2 M, 5×10−3 M, 10−3 M, 5×10−4 M, 10−4 M, 5×10−5 M, 10−5 M, 5×10−6 M, 10−6 M, 5×10−7 M, 10−7 M, 5×10−8 M, 10−8 M, 5×10−9 M, 10−9 M, 5×10−10 M, 10−10 M, 5×10−11 M, 10−11 M, 5×10−12 M, 10−12 M, 5×10−13 M, 10−13 M, 5×10−14 M, 10−14 M, 5×10−15 M, or 10−15 M.

Antigen-binding fragments of a binding molecule or antibody as provided herein including single-chain antibodies or other antigen-binding domains that can exist alone or in combination with one or more of the following: hinge region, CH1, CH2, CH3, or CH4 domains, J-chain, or secretory component. Also included are antigen-binding fragments that can include any combination of variable region(s) with one or more of a hinge region, CH1, CH2, CH3, or CH4 domains, a J-chain, or a secretory component. Binding molecules, e.g., antibodies, or antigen-binding fragments thereof can be from any animal origin including birds and mammals. The antibodies can be human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in, U.S. Pat. No. 5,939,598 by Kucherlapati et al.

As used herein, the term “heavy chain subunit” includes amino acid sequences derived from an immunoglobulin heavy chain, a binding molecule, e.g., an antibody comprising a heavy chain subunit can include at least one of: a VH domain, a CH1 domain, a hinge (e.g., upper, middle, and/or lower hinge region) domain, a CH2 domain, a CH3 domain, a CH4 domain, or a variant or fragment thereof. For example, a binding molecule, e.g., an antibody or fragment, variant, or derivative thereof can include without limitation, in addition to a VH domain:, a CH1 domain; a CH1 domain, a hinge, and a CH2 domain; a CH1 domain and a CH3 domain; a CH1 domain, a hinge, and a CH3 domain; or a CH1 domain, a hinge domain, a CH2 domain, and a CH3 domain. In certain aspects a binding molecule, e.g., an antibody or fragment, variant, or derivative thereof can include, in addition to a VH domain, a CH3 domain and a CH4 domain; or a CH3 domain, a CH4 domain, and a J-chain. Further, a binding molecule for use in the disclosure can lack certain constant region portions, e.g., all or part of a CH2 domain. It will be understood by one of ordinary skill in the art that these domains (e.g., the heavy chain subunit) can be modified such that they vary in amino acid sequence from the original immunoglobulin molecule.

As used herein, the term “light chain subunit” includes amino acid sequences derived from an immunoglobulin light chain The light chain subunit includes at least a VL, and can further include a CL (e.g., Cκ or Cλ) domain.

Binding molecules, e.g., antibodies or antigen-binding fragments, variants, or derivatives thereof can be described or specified in terms of the epitope(s) or portion(s) of an antigen that they recognize or specifically bind. The portion of a target antigen that specifically interacts with the antigen-binding domain of an antibody is an “epitope,” or an “antigenic determinant.” A target antigen can comprise a single epitope or at least two epitopes, and can include any number of epitopes, depending on the size, conformation, and type of antigen.

As previously indicated, the subunit structures and three-dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of a typical IgG heavy chain molecule.

As used herein the term “CH2 domain” includes the portion of a heavy chain molecule that extends, e.g., from about amino acid 244 to amino acid 360 of an IgG antibody using conventional numbering schemes (amino acids 244 to 360, Kabat numbering system; and amino acids 231-340, EU numbering system; see Kabat E A et al., op. cit. The CH3 domain extends from the CH2 domain to the C-terminal of the IgG molecule and comprises approximately 108 amino acids. Certain immunoglobulin classes, e.g., IgM, further include a CH4 region.

As used herein, the term “hinge region” includes the portion of a heavy chain molecule that joins the CH1 domain to the CH2 domain in IgG, IgA, and IgD heavy chains. This hinge region comprises approximately 25 amino acids and is flexible, thus allowing the two N-terminal antigen binding regions to move independently.

As used herein the term “disulfide bond” includes the covalent bond formed between two sulfur atoms. The amino acid cysteine comprises a thiol group that can form a disulfide bond or bridge with a second thiol group.

As used herein, the term “chimeric antibody” refers to an antibody in which the immunoreactive region or site is obtained or derived from a first species and the constant region (which can be intact, partial or modified) is obtained from a second species. In some embodiments the target binding region or site will be from a non-human source (e.g. mouse or primate) and the constant region is human.

The terms “multispecific antibody” or “bispecific antibody” refer to an antibody that has antigen-binding domains for two or more different epitopes within a single antibody molecule. Other binding molecules in addition to the canonical antibody structure can be constructed with two binding specificities. Epitope binding by bispecific or multispecific antibodies can be simultaneous or sequential.

As used herein, the term “engineered antibody” refers to an antibody in which the variable domain in either the heavy and light chain or both is altered by at least partial replacement of one or more amino acids in either the CDR or framework regions. In certain aspects entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain aspects, not all of the CDRs are replaced with the complete CDRs from the donor variable region and yet the antigen binding capacity of the donor can still be transferred to the recipient variable domains. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly, a portion of a polypeptide that is “carboxy-terminal” or “C-terminal” to another portion of a polypeptide is that portion that comes later in the sequential polypeptide chain. For example, in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

Terms such as “treating” or “treatment” or “to treat” or “alleviating” or “to alleviate” refer to therapeutic measures that cure, slow down, lessen symptoms of, and/or halt or slow the progression of an existing diagnosed pathologic condition or disorder. Terms such as “prevent,” “prevention,” “avoid,” “deterrence” and the like refer to prophylactic or preventative measures that prevent the development of an undiagnosed targeted pathologic condition or disorder. Thus, “those in need of treatment” can include those already with the disorder; those prone to have the disorder; and those in whom the disorder is to be prevented.

As used herein the terms “serum half-life” or “plasma half-life” refer to the time it takes (e.g., in minutes, hours, or days) following administration for the serum or plasma concentration of a drug, e.g., a binding molecule such as an antibody or fragment thereof as described herein, to be reduced by 50%. Two half-lives can be described: the alpha half-life or a half-life, which is the rate of decline in plasma concentrations due to the process of drug redistribution from the central compartment, e.g., the blood in the case of intravenous delivery, to a peripheral compartment (e.g., a tissue or organ), and the beta half-life or β half-life, which is the rate of decline due to the processes of excretion or metabolism.

As used herein the term “area under the plasma drug concentration-time curve” or “AUC” reflects the actual body exposure to drug after administration of a dose of the drug and is expressed in mg*h/L. This area under the curve is dependent on the rate of elimination of the drug from the body and the dose administered.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, phrases such as “a subject that would benefit from therapy” and “an animal in need of treatment” refers to a subset of subjects, from amongst all prospective subjects, which would benefit from administration of a given therapeutic agent, e.g., a binding molecule such as an antibody, comprising one or more antigen-binding domains. Such binding molecules, e.g., antibodies, can be used, e.g., for a diagnostic procedure and/or for treatment or prevention of a disease.

As used herein, the terms “TNF superfamily receptor proteins,” “TNFSFR,” “TNF receptor family,” “TNF receptors” or any combination of such phrases, refer to the family of Tumor Necrosis Factor transmembrane receptor proteins expressed on the surface of various cells and tissues. Family members of this superfamily include those that, upon activation by ligand binding or agonist antibody binding can trigger: activation, an inflammatory response, apoptosis (or inhibit apoptosis), proliferation, and/or morphogenesis in a cell in which the receptor protein is expressed. TNFSFRs include, but are not limited to TNFR1 (DR1), TNFR2, TNFR1/2, CD40 (p50), Fas (CD95, Apo 1, DR2), CD30, 4-1BB (CD137, ILA), TRAILR1 (DR4, Apo2), TRAILR2 (DR5), TRAILR3 (DcR1), TRAILR4 (DcR2), OPG (OCIF), TWEAKR (FN14), LIGHTR (HVEM), DcR3, DR3, EDAR, XEDAR, LT-(3R, GITR (AITR), TACI, BCMA, CD27, 0X40 (CD134), RANK (TRANCER), RELT, and BAFF-R. See, e.g., Wajant, H. Cell Death and Differentiation 22:1727-1741 (2015).

Disclosed herein are certain binding molecules, or antigen-binding fragments, variants, or derivatives thereof that bind to the TNFSFR GITR. Disclosed herein are certain binding molecules, or antigen-binding fragments, variants, or derivatives thereof that agonistically bind to the TNFSFR GITR, and can thereby elicit, e.g., proliferation and enhanced effector function in activated CTLs expressing GITR, and impairment of immune suppression by CD25+ CD4+ FoxP3+ Tregs, e.g., in the microenvironment surrounding a tumor, thus promoting anti-tumor immunity. Unless specifically referring to full-sized antibodies, the term “binding molecule” encompasses full-sized antibodies as well as antigen-binding subunits, fragments, variants, analogs, or derivatives of such antibodies, e.g., engineered antibody molecules or fragments that bind antigen in a manner similar to antibody molecules, but which use a different scaffold.

The precursor form of isoform 1 of human GITR comprises the amino acid sequence SEQ ID NO: 196 (UniProtKB/Swiss-Prot: 035714.1). Other isoforms share significant homology with SEQ ID NO: 196. The mature protein includes amino acids 26 to 241 of SEQ ID NO: 196, with amino acids 1-25 comprising the signal peptide. The extracellular domain of human GITR includes amino acids 26 to 162 of SEQ ID NO: 196. The transmembrane domain of human GITR includes amino acids 163 to 183 of SEQ ID NO: 196. The cytoplasmic domain of human GITR includes amino acids 184 to 241 of SEQ ID NO: 196.

SEQ ID NO: 196:
MAQHGAMGAFRALCGLALLCALSLGQRPTGGPGCGPGRLL
LGTGTDARCCRVHTTRCCRDYPGEECCSEWDCMCVQPEFH
CGDPCCTTCRHHPCPPGQGVQSQGKFSFGFQCIDCASGTF
SGGHEGHCKPWTDCTQFGFLTVFPGNKTHNAVCVPGSPPA
EPLGWLTVVLLAVAACVLLLTSAQLGLHIWQLRSQCMWPR
ETQLLLEVPPSTEDARSCQFPEEERGERSAEEKGRLGDLW
V

The predicted precursor form of cynomolgus monkey GITR comprises the amino acid sequence SEQ ID NO: 395 (GenBank Accession No. XP_005545180.1). The mature protein includes amino acids 20 to 235 of SEQ ID NO: 395, with amino acids 1-19 comprising the signal peptide.

SEQ ID NO: 395:
MCACGTLCCLALLCAASLGQRPTGGPGCGPGRLLLGTGKD
ARCCRVHPTRCCRDYQSEECCSEWDCVCVQPEFHCGNPCC
TTCQHHPCPSGQGVQPQGKFSFGFRCVDCALGTFSRGHDG
HCKPWTDCTQFGFLTVFPGNKTHNAVCVPGSPPAEPPGWL
TIVLLAVAACVLLLTSAQLGLHIWQLGSQPTGPRETQLLL
EVPPSTEDASSCQFPEEERGERLAEEKGRLGDLWV

The precursor form of murine GITR comprises the amino acid sequence SEQ ID NO: 197 (UniProtKB/Swiss-Prot: 035714.1). Other isoforms share significant homology with SEQ ID NO: 197. The mature protein includes amino acids 20 to 228 of SEQ ID NO: 197, with amino acids 1-19 comprising the signal peptide. The extracellular domain of murine GITR includes amino acids 20 to 153 of SEQ ID NO: 197. The transmembrane domain of murine GITR includes amino acids 154 to 174 of SEQ ID NO: 197. The cytoplasmic domain of murine GITR includes amino acids 175 to 228 of SEQ ID NO: 197.

SEQ ID NO: 197:
MGAWAMLYGVSMLCVLDLGQPSVVEEPGCGPGKVQNGSG
NNTRCCSLYAPGKEDCPKERCICVTPEYHCGDPQCKICK
HYPCQPGQRVESQGDIVFGFRCVACAMGTFSAGRDGHCR
LWTNCSQFGFLTMFPGNKTHNAVCIPEPLPTEQYGHLTV
IFLVMAACIFFLTTVQLGLHIWQLRRQHMCPRETQPFAE
VQLSAEDACSFQFPEEERGEQTEEKCHLGGRWP

### Anti-GITR Antigen-Binding Domains

This disclosure provides an antigen-binding domain that specifically binds to glucocorticoid-induced TNF receptor (GITR). The provided antigen-binding domain includes a heavy chain variable region (VH) and light chain variable region (VL), where the VH and VL include six immunoglobulin complementarity determining regions HCDR1, HCDR2, HCDR3, LCDR1, LCDR2, and LCDR3, comprising, respectively, the amino acid sequences SEQ ID NO: 2, SEQ ID NO: 3, SEQ ID NO: 4, SEQ ID NO: 6, SEQ ID NO: 7, and SEQ ID NO: 8; SEQ ID NO: 10, SEQ ID NO: 11, SEQ ID NO: 12, SEQ ID NO: 14, SEQ ID NO: 15, and SEQ ID NO: 16; SEQ ID NO: 18, SEQ ID NO: 19, SEQ ID NO: 20, SEQ ID NO: 22, SEQ ID NO: 23, and SEQ ID NO: 24; SEQ ID NO: 26, SEQ ID NO: 27, SEQ ID NO: 28, SEQ ID NO: 30, SEQ ID NO: 31, and SEQ ID NO: 32; SEQ ID NO: 34, SEQ ID NO: 35, SEQ ID NO: 36, SEQ ID NO: 38, SEQ ID NO: 39, and SEQ ID NO: 40; SEQ ID NO: 42, SEQ ID NO: 43, SEQ ID NO: 44, SEQ ID NO: 46, SEQ ID NO: 47, and SEQ ID NO: 48; SEQ ID NO: 50, SEQ ID NO: 51, SEQ ID NO: 52, SEQ ID NO: 54, SEQ ID NO: 55, and SEQ ID NO: 56; SEQ ID NO: 58, SEQ ID NO: 59, SEQ ID NO: 60, SEQ ID NO: 62, SEQ ID NO: 63, and SEQ ID NO: 64; SEQ ID NO: 66, SEQ ID NO: 67, SEQ ID NO: 68, SEQ ID NO: 70, SEQ ID NO: 71, and SEQ ID NO: 72; SEQ ID NO: 74, SEQ ID NO: 75, SEQ ID NO: 76, SEQ ID NO: 78, SEQ ID NO: 79, and SEQ ID NO: 80; SEQ ID NO: 82, SEQ ID NO: 83, SEQ ID NO: 84, SEQ ID NO: 86, SEQ ID NO: 87, and SEQ ID NO: 88; SEQ ID NO: 90, SEQ ID NO: 91, SEQ ID NO: 92, SEQ ID NO: 94, SEQ ID NO: 95, and SEQ ID NO: 96; SEQ ID NO: 98, SEQ ID NO: 99, SEQ ID NO: 100, SEQ ID NO: 102, SEQ ID NO: 103, and SEQ ID NO: 104; SEQ ID NO: 106, SEQ ID NO: 107, SEQ ID NO: 108, SEQ ID NO: 110, SEQ ID NO: 111, and SEQ ID NO: 112; SEQ ID NO: 114, SEQ ID NO: 115, SEQ ID NO: 116, SEQ ID NO: 118, SEQ ID NO: 119, and SEQ ID NO: 120; SEQ ID NO: 122, SEQ ID NO: 123, SEQ ID NO: 124, SEQ ID NO: 126, SEQ ID NO: 127, and SEQ ID NO: 128; SEQ ID NO: 130, SEQ ID NO: 131, SEQ ID NO: 132, SEQ ID NO: 134, SEQ ID NO: 135, and SEQ ID NO: 136; SEQ ID NO: 138, SEQ ID NO: 139, SEQ ID NO: 140, SEQ ID NO: 142, SEQ ID NO: 143, and SEQ ID NO: 144; SEQ ID NO: 146, SEQ ID NO: 147, SEQ ID NO: 148, SEQ ID NO: 150, SEQ ID NO: 151, and SEQ ID NO: 152; SEQ ID NO: 154, SEQ ID NO: 155, SEQ ID NO: 156, SEQ ID NO: 158, SEQ ID NO: 159, and SEQ ID NO: 160; SEQ ID NO: 162, SEQ ID NO: 163, SEQ ID NO: 164, SEQ ID NO: 166, SEQ ID NO: 167, and SEQ ID NO: 168; SEQ ID NO: 170, SEQ ID NO: 171, SEQ ID NO: 172, SEQ ID NO: 174, SEQ ID NO: 175, and SEQ ID NO: 176; SEQ ID NO: 178, SEQ ID NO: 179, SEQ ID NO: 180, SEQ ID NO: 182, SEQ ID NO: 183, and SEQ ID NO: 184; SEQ ID NO: 186, SEQ ID NO: 187, SEQ ID NO: 188, SEQ ID NO: 190, SEQ ID NO: 191, and SEQ ID NO: 192; SEQ ID NO: 204, SEQ ID NO: 205, SEQ ID NO: 206, SEQ ID NO: 208, SEQ ID NO: 209, and SEQ ID NO: 210; SEQ ID NO: 212, SEQ ID NO: 213, SEQ ID NO: 214, SEQ ID NO: 216, SEQ ID NO: 217, and SEQ ID NO: 218; SEQ ID NO: 220, SEQ ID NO: 221, SEQ ID NO: 222, SEQ ID NO: 224, SEQ ID NO: 225, and SEQ ID NO: 226; SEQ ID NO: 228, SEQ ID NO: 229, SEQ ID NO: 230, SEQ ID NO: 232, SEQ ID NO: 233, and SEQ ID NO: 234; SEQ ID NO: 236, SEQ ID NO: 237, SEQ ID NO: 238, SEQ ID NO: 240, SEQ ID NO: 241, and SEQ ID NO: 242; SEQ ID NO: 244, SEQ ID NO: 245, SEQ ID NO: 246, SEQ ID NO: 248, SEQ ID NO: 249, and SEQ ID NO: 250; SEQ ID NO: 252, SEQ ID NO: 253, SEQ ID NO: 254, SEQ ID NO: 256, SEQ ID NO: 257, and SEQ ID NO: 258; SEQ ID NO: 260, SEQ ID NO: 261, SEQ ID NO: 262, SEQ ID NO: 264, SEQ ID NO: 265, and SEQ ID NO: 266; SEQ ID NO: 268, SEQ ID NO: 269, SEQ ID NO: 270, SEQ ID NO: 272, SEQ ID NO: 273, and SEQ ID NO: 274; SEQ ID NO: 276, SEQ ID NO: 277, SEQ ID NO: 278, SEQ ID NO: 280, SEQ ID NO: 281, and SEQ ID NO: 282; SEQ ID NO: 284, SEQ ID NO: 285, SEQ ID NO: 286, SEQ ID NO: 288, SEQ ID NO: 289, and SEQ ID NO: 290; SEQ ID NO: 292, SEQ ID NO: 293, SEQ ID NO: 294, SEQ ID NO: 296, SEQ ID NO: 297, and SEQ ID NO: 298; SEQ ID NO: 300, SEQ ID NO: 301, SEQ ID NO: 302, SEQ ID NO: 304, SEQ ID NO: 305, and SEQ ID NO: 306; SEQ ID NO: 308, SEQ ID NO: 309, SEQ ID NO: 310, SEQ ID NO: 312, SEQ ID NO: 313, and SEQ ID NO: 314; SEQ ID NO: 316, SEQ ID NO: 317, SEQ ID NO: 318, SEQ ID NO: 320, SEQ ID NO: 321, and SEQ ID NO: 322; SEQ ID NO: 324, SEQ ID NO: 325, SEQ ID NO: 326, SEQ ID NO: 328, SEQ ID NO: 329, and SEQ ID NO: 330; SEQ ID NO: 332, SEQ ID NO: 333, SEQ ID NO: 334, SEQ ID NO: 336, SEQ ID NO: 337, and SEQ ID NO: 338; SEQ ID NO: 340, SEQ ID NO: 341, SEQ ID NO: 342, SEQ ID NO: 344, SEQ ID NO: 345, and SEQ ID NO: 346; SEQ ID NO: 348, SEQ ID NO: 349, SEQ ID NO: 350, SEQ ID NO: 352, SEQ ID NO: 353, and SEQ ID NO: 354; SEQ ID NO: 356, SEQ ID NO: 357, SEQ ID NO: 358, SEQ ID NO: 360, SEQ ID NO: 361, and SEQ ID NO: 362; SEQ ID NO: 364, SEQ ID NO: 365, SEQ ID NO: 366, SEQ ID NO: 368, SEQ ID NO: 369, and SEQ ID NO: 370; SEQ ID NO: 372, SEQ ID NO: 373, SEQ ID NO: 374, SEQ ID NO: 376, SEQ ID NO: 377, and SEQ ID NO: 378; SEQ ID NO: 380, SEQ ID NO: 381, SEQ ID NO: 382, SEQ ID NO: 384, SEQ ID NO: 385, and SEQ ID NO: 386; or SEQ ID NO: 388, SEQ ID NO: 389, SEQ ID NO: 390, SEQ ID NO: 392, SEQ ID NO: 393, and SEQ ID NO: 394; or the amino acid sequences SEQ ID NO: 2, SEQ ID NO: 3, SEQ ID NO: 4, SEQ ID NO: 6, SEQ ID NO: 7, and SEQ ID NO: 8; SEQ ID NO: 10, SEQ ID NO: 11, SEQ ID NO: 12, SEQ ID NO: 14, SEQ ID NO: 15, and SEQ ID NO: 16; SEQ ID NO: 18, SEQ ID NO: 19, SEQ ID NO: 20, SEQ ID NO: 22, SEQ ID NO: 23, and SEQ ID NO: 24; SEQ ID NO: 26, SEQ ID NO: 27, SEQ ID NO: 28, SEQ ID NO: 30, SEQ ID NO: 31, and SEQ ID NO: 32; SEQ ID NO: 34, SEQ ID NO: 35, SEQ ID NO: 36, SEQ ID NO: 38, SEQ ID NO: 39, and SEQ ID NO: 40; SEQ ID NO: 42, SEQ ID NO: 43, SEQ ID NO: 44, SEQ ID NO: 46, SEQ ID NO: 47, and SEQ ID NO: 48; SEQ ID NO: 50, SEQ ID NO: 51, SEQ ID NO: 52, SEQ ID NO: 54, SEQ ID NO: 55, and SEQ ID NO: 56; SEQ ID NO: 58, SEQ ID NO: 59, SEQ ID NO: 60, SEQ ID NO: 62, SEQ ID NO: 63, and SEQ ID NO: 64; SEQ ID NO: 66, SEQ ID NO: 67, SEQ ID NO: 68, SEQ ID NO: 70, SEQ ID NO: 71, and SEQ ID NO: 72; SEQ ID NO: 74, SEQ ID NO: 75, SEQ ID NO: 76, SEQ ID NO: 78, SEQ ID NO: 79, and SEQ ID NO: 80; SEQ ID NO: 82, SEQ ID NO: 83, SEQ ID NO: 84, SEQ ID NO: 86, SEQ ID NO: 87, and SEQ ID NO: 88; SEQ ID NO: 90, SEQ ID NO: 91, SEQ ID NO: 92, SEQ ID NO: 94, SEQ ID NO: 95, and SEQ ID NO: 96; SEQ ID NO: 98, SEQ ID NO: 99, SEQ ID NO: 100, SEQ ID NO: 102, SEQ ID NO: 103, and SEQ ID NO: 104; SEQ ID NO: 106, SEQ ID NO: 107, SEQ ID NO: 108, SEQ ID NO: 110, SEQ ID NO: 111, and SEQ ID NO: 112; SEQ ID NO: 114, SEQ ID NO: 115, SEQ ID NO: 116, SEQ ID NO: 118, SEQ ID NO: 119, and SEQ ID NO: 120; SEQ ID NO: 122, SEQ ID NO: 123, SEQ ID NO: 124, SEQ ID NO: 126, SEQ ID NO: 127, and SEQ ID NO: 128; SEQ ID NO: 130, SEQ ID NO: 131, SEQ ID NO: 132, SEQ ID NO: 134, SEQ ID NO: 135, and SEQ ID NO: 136; SEQ ID NO: 138, SEQ ID NO: 139, SEQ ID NO: 140, SEQ ID NO: 142, SEQ ID NO: 143, and SEQ ID NO: 144; SEQ ID NO: 146, SEQ ID NO: 147, SEQ ID NO: 148, SEQ ID NO: 150, SEQ ID NO: 151, and SEQ ID NO: 152; SEQ ID NO: 154, SEQ ID NO: 155, SEQ ID NO: 156, SEQ ID NO: 158, SEQ ID NO: 159, and SEQ ID NO: 160; SEQ ID NO: 162, SEQ ID NO: 163, SEQ ID NO: 164, SEQ ID NO: 166, SEQ ID NO: 167, and SEQ ID NO: 168; SEQ ID NO: 170, SEQ ID NO: 171, SEQ ID NO: 172, SEQ ID NO: 174, SEQ ID NO: 175, and SEQ ID NO: 176; SEQ ID NO: 178, SEQ ID NO: 179, SEQ ID NO: 180, SEQ ID NO: 182, SEQ ID NO: 183, and SEQ ID NO: 184; SEQ ID NO: 186, SEQ ID NO: 187, SEQ ID NO: 188, SEQ ID NO: 190, SEQ ID NO: 191, and SEQ ID NO: 192; SEQ ID NO: 204, SEQ ID NO: 205, SEQ ID NO: 206, SEQ ID NO: 208, SEQ ID NO: 209, and SEQ ID NO: 210; SEQ ID NO: 212, SEQ ID NO: 213, SEQ ID NO: 214, SEQ ID NO: 216, SEQ ID NO: 217, and SEQ ID NO: 218; SEQ ID NO: 220, SEQ ID NO: 221, SEQ ID NO: 222, SEQ ID NO: 224, SEQ ID NO: 225, and SEQ ID NO: 226; SEQ ID NO: 228, SEQ ID NO: 229, SEQ ID NO: 230, SEQ ID NO: 232, SEQ ID NO: 233, and SEQ ID NO: 234; SEQ ID NO: 236, SEQ ID NO: 237, SEQ ID NO: 238, SEQ ID NO: 240, SEQ ID NO: 241, and SEQ ID NO: 242; SEQ ID NO: 244, SEQ ID NO: 245, SEQ ID NO: 246, SEQ ID NO: 248, SEQ ID NO: 249, and SEQ ID NO: 250; SEQ ID NO: 252, SEQ ID NO: 253, SEQ ID NO: 254, SEQ ID NO: 256, SEQ ID NO: 257, and SEQ ID NO: 258; SEQ ID NO: 260, SEQ ID NO: 261, SEQ ID NO: 262, SEQ ID NO: 264, SEQ ID NO: 265, and SEQ ID NO: 266; SEQ ID NO: 268, SEQ ID NO: 269, SEQ ID NO: 270, SEQ ID NO: 272, SEQ ID NO: 273, and SEQ ID NO: 274; SEQ ID NO: 276, SEQ ID NO: 277, SEQ ID NO: 278, SEQ ID NO: 280, SEQ ID NO: 281, and SEQ ID NO: 282; SEQ ID NO: 284, SEQ ID NO: 285, SEQ ID NO: 286, SEQ ID NO: 288, SEQ ID NO: 289, and SEQ ID NO: 290; SEQ ID NO: 292, SEQ ID NO: 293, SEQ ID NO: 294, SEQ ID NO: 296, SEQ ID NO: 297, and SEQ ID NO: 298; SEQ ID NO: 300, SEQ ID NO: 301, SEQ ID NO: 302, SEQ ID NO: 304, SEQ ID NO: 305, and SEQ ID NO: 306; SEQ ID NO: 308, SEQ ID NO: 309, SEQ ID NO: 310, SEQ ID NO: 312, SEQ ID NO: 313, and SEQ ID NO: 314; SEQ ID NO: 316, SEQ ID NO: 317, SEQ ID NO: 318, SEQ ID NO: 320, SEQ ID NO: 321, and SEQ ID NO: 322; SEQ ID NO: 324, SEQ ID NO: 325, SEQ ID NO: 326, SEQ ID NO: 328, SEQ ID NO: 329, and SEQ ID NO: 330; SEQ ID NO: 332, SEQ ID NO: 333, SEQ ID NO: 334, SEQ ID NO: 336, SEQ ID NO: 337, and SEQ ID NO: 338; SEQ ID NO: 340, SEQ ID NO: 341, SEQ ID NO: 342, SEQ ID NO: 344, SEQ ID NO: 345, and SEQ ID NO: 346; SEQ ID NO: 348, SEQ ID NO: 349, SEQ ID NO: 350, SEQ ID NO: 352, SEQ ID NO: 353, and SEQ ID NO: 354; SEQ ID NO: 356, SEQ ID NO: 357, SEQ ID NO: 358, SEQ ID NO: 360, SEQ ID NO: 361, and SEQ ID NO: 362; SEQ ID NO: 364, SEQ ID NO: 365, SEQ ID NO: 366, SEQ ID NO: 368, SEQ ID NO: 369, and SEQ ID NO: 370; SEQ ID NO: 372, SEQ ID NO: 373, SEQ ID NO: 374, SEQ ID NO: 376, SEQ ID NO: 377, and SEQ ID NO: 378; SEQ ID NO: 380, SEQ ID NO: 381, SEQ ID NO: 382, SEQ ID NO: 384, SEQ ID NO: 385, and SEQ ID NO: 386; or SEQ ID NO: 388, SEQ ID NO: 389, SEQ ID NO: 390, SEQ ID NO: 392, SEQ ID NO: 393, and SEQ ID NO: 394, except for one, two, three, or four amino acid substitutions in one or more of the CDRs. In certain aspects, the provided antigen binding domain can be included in an antibody or antigen-binding fragment, variant, or derivative thereof. In certain aspects the antibody is a multimeric, e.g., a dimeric pentameric, or hexameric anti-GITR antibody as described elsewhere herein. In certain aspects the VH of the antigen-binding domain further comprises framework regions (HFWs) HFW1, HFW2, HFW3, and HFW4, and the VL of the antigen-binding domain further comprises framework regions (LFWs) LFW1, LFW2, LFW3, and LFW4. In certain aspects the framework regions are derived from a human antibody. In certain aspects the framework regions are derived from a non-human antibody. In certain aspects the VH of the provided anti-GITR antigen-binding domain comprises the amino acid sequence SEQ ID NO: 1, SEQ ID NO: 9, SEQ ID NO: 17, SEQ ID NO: 25, SEQ ID NO: 33, SEQ ID NO: 41, SEQ ID NO: 49, SEQ ID NO: 57, SEQ ID NO: 65, SEQ ID NO: 73, SEQ ID NO: 81, SEQ ID NO: 89, SEQ ID NO: 97, SEQ ID NO: 105, SEQ ID NO: 113, SEQ ID NO: 121, SEQ ID NO: 129, SEQ ID NO: 137, SEQ ID NO: 145, SEQ ID NO: 153, SEQ ID NO: 161, SEQ ID NO: 169, SEQ ID NO: 177, SEQ ID NO: 185, SEQ ID NO: 203, SEQ ID NO: 211, SEQ ID NO: 219, SEQ ID NO: 227, SEQ ID NO: 235, SEQ ID NO: 243, SEQ ID NO: 251, SEQ ID NO: 259, SEQ ID NO: 267, SEQ ID NO: 275, SEQ ID NO: 283, SEQ ID NO: 291, SEQ ID NO: 299, SEQ ID NO: 307, SEQ ID NO: 315, SEQ ID NO: 323, SEQ ID NO: 331, SEQ ID NO: 339, SEQ ID NO: 347, SEQ ID NO: 355, SEQ ID NO: 363, SEQ ID NO: 371, SEQ ID NO: 379, or SEQ ID NO: 387. In certain aspects the VL of the provided anti-GITR antigen-binding domain comprises the amino acid sequence SEQ ID NO: 5, SEQ ID NO: 13, SEQ ID NO: 21, SEQ ID NO: 29, SEQ ID NO: 37, SEQ ID NO: 45, SEQ ID NO: 53, SEQ ID NO: 61, SEQ ID NO: 69, SEQ ID NO: 77, SEQ ID NO: 85, SEQ ID NO: 93, SEQ ID NO: 101, SEQ ID NO: 109, SEQ ID NO: 117, SEQ ID NO: 125, SEQ ID NO: 133, SEQ ID NO: 141, SEQ ID NO: 149, SEQ ID NO: 157, SEQ ID NO: 165, SEQ ID NO: 173, SEQ ID NO: 181, SEQ ID NO: 189, SEQ ID NO: 207, SEQ ID NO: 215, SEQ ID NO: 223, SEQ ID NO: 231, SEQ ID NO: 239, SEQ ID NO: 247, SEQ ID NO: 255, SEQ ID NO: 263, SEQ ID NO: 271, SEQ ID NO: 279, SEQ ID NO: 287, SEQ ID NO: 295, SEQ ID NO: 303, SEQ ID NO: 311, SEQ ID NO: 319, SEQ ID NO: 327, SEQ ID NO: 335, SEQ ID NO: 343, SEQ ID NO: 351, SEQ ID NO: 359, SEQ ID NO: 367, SEQ ID NO: 375, SEQ ID NO: 383, or SEQ ID NO: 391. In certain aspects the VH and VL comprise, respectively, the amino acid sequences SEQ ID NO: 1 and SEQ ID NO: 5, SEQ ID NO: 9 and SEQ ID NO: 13, SEQ ID NO: 17 and SEQ ID NO: 21, SEQ ID NO: 25 and SEQ ID NO: 29, SEQ ID NO: 33 and SEQ ID NO: 37, SEQ ID NO: 41 and SEQ ID NO: 45, SEQ ID NO: 49 and SEQ ID NO: 53, SEQ ID NO: 57 and SEQ ID NO: 61, SEQ ID NO: 65 and SEQ ID NO: 69, SEQ ID NO: 73 and SEQ ID NO: 77, SEQ ID NO: 81 and SEQ ID NO: 85, SEQ ID NO: 89 and SEQ ID NO: 93, SEQ ID NO: 97 and SEQ ID NO: 101, SEQ ID NO: 105 and SEQ ID NO: 109, SEQ ID NO: 113 and SEQ ID NO: 117, SEQ ID NO: 121 and SEQ ID NO: 125, SEQ ID NO: 129 and SEQ ID NO: 133, SEQ ID NO: 137 and SEQ ID NO: 141, SEQ ID NO: 145 and SEQ ID NO: 149, SEQ ID NO: 153 and SEQ ID NO: 157, SEQ ID NO: 161 and SEQ ID NO: 165, SEQ ID NO: 169 and SEQ ID NO: 173, SEQ ID NO: 177 and SEQ ID NO: 181, SEQ ID NO: 185 and SEQ ID NO: 189, SEQ ID NO: 203 and SEQ ID NO: 207, SEQ ID NO: 211 and SEQ ID NO: 215, SEQ ID NO: 219 and SEQ ID NO: 223, SEQ ID NO: 227 and SEQ ID NO: 231, SEQ ID NO: 235 and SEQ ID NO: 239, SEQ ID NO: 243 and SEQ ID NO: 247, SEQ ID NO: 251 and SEQ ID NO: 255, SEQ ID NO: 259 and SEQ ID NO: 263, SEQ ID NO: 267 and SEQ ID NO: 271, SEQ ID NO: 275 and SEQ ID NO: 279, SEQ ID NO: 283 and SEQ ID NO: 287, SEQ ID NO: 291 and SEQ ID NO: 295, SEQ ID NO: 299 and SEQ ID NO: 303, SEQ ID NO: 307 and SEQ ID NO: 311, SEQ ID NO: 315 and SEQ ID NO: 319, SEQ ID NO: 323 and SEQ ID NO: 327, SEQ ID NO: 331 and SEQ ID NO: 335, SEQ ID NO: 339 and SEQ ID NO: 343, SEQ ID NO: 347 and SEQ ID NO: 351, SEQ ID NO: 355 and SEQ ID NO: 359, SEQ ID NO: 363 and SEQ ID NO: 367, SEQ ID NO: 371 and SEQ ID NO: 375, SEQ ID NO: 379 and SEQ ID NO: 383, or SEQ ID NO: 387 and SEQ ID NO: 391.

This disclosure also provides an antigen-binding domain that specifically binds to glucocorticoid-induced TNF receptor (GITR). The provided antigen-binding domain includes a heavy chain variable region (VH) and light chain variable region (VL), where the VH comprises an amino acid sequence at least 80%, 85%, 90%, 95%, or 100% identical to SEQ ID NO: 1, SEQ ID NO: 9, SEQ ID NO: 17, SEQ ID NO: 25, SEQ ID NO: 33, SEQ ID NO: 41, SEQ ID NO: 49, SEQ ID NO: 57, SEQ ID NO: 65, SEQ ID NO: 73, SEQ ID NO: 81, SEQ ID NO: 89, SEQ ID NO: 97, SEQ ID NO: 105, SEQ ID NO: 113, SEQ ID NO: 121, SEQ ID NO: 129, SEQ ID NO: 137, SEQ ID NO: 145, SEQ ID NO: 153, SEQ ID NO: 161, SEQ ID NO: 169, SEQ ID NO: 177, SEQ ID NO: 185, SEQ ID NO: 203, SEQ ID NO: 211, SEQ ID NO: 219, SEQ ID NO: 227, SEQ ID NO: 235, SEQ ID NO: 243, SEQ ID NO: 251, SEQ ID NO: 259, SEQ ID NO: 267, SEQ ID NO: 275, SEQ ID NO: 283, SEQ ID NO: 291, SEQ ID NO: 299, SEQ ID NO: 307, SEQ ID NO: 315, SEQ ID NO: 323, SEQ ID NO: 331, SEQ ID NO: 339, SEQ ID NO: 347, SEQ ID NO: 355, SEQ ID NO: 363, SEQ ID NO: 371, SEQ ID NO: 379, or SEQ ID NO: 387. In certain aspects the VL comprises an amino acid sequence at least 80%, 85%, 90%, 95%, or 100% identical to SEQ ID NO: 5, SEQ ID NO: 13, SEQ ID NO: 21, SEQ ID NO: 29, SEQ ID NO: 37, SEQ ID NO: 45, SEQ ID NO: 53, SEQ ID NO: 61, SEQ ID NO: 69, SEQ ID NO: 77, SEQ ID NO: 85, SEQ ID NO: 93, SEQ ID NO: 101, SEQ ID NO: 109, SEQ ID NO: 117, SEQ ID NO: 125, SEQ ID NO: 133, SEQ ID NO: 141, SEQ ID NO: 149, SEQ ID NO: 157, SEQ ID NO: 165, SEQ ID NO: 173, SEQ ID NO: 181, SEQ ID NO: 189, SEQ ID NO: 207, SEQ ID NO: 215, SEQ ID NO: 223, SEQ ID NO: 231, SEQ ID NO: 239, SEQ ID NO: 247, SEQ ID NO: 255, SEQ ID NO: 263, SEQ ID NO: 271, SEQ ID NO: 279, SEQ ID NO: 287, SEQ ID NO: 295, SEQ ID NO: 303, SEQ ID NO: 311, SEQ ID NO: 319, SEQ ID NO: 327, SEQ ID NO: 335, SEQ ID NO: 343, SEQ ID NO: 351, SEQ ID NO: 359, SEQ ID NO: 367, SEQ ID NO: 375, SEQ ID NO: 383, or SEQ ID NO: 391. In certain aspects, the provided antigen binding domain can be included in an antibody or antigen-binding fragment, variant, or derivative thereof. In certain aspects the antibody is a multimeric, e.g., a dimeric pentameric, or hexameric anti-GITR antibody as described elsewhere herein.

This disclosure also provides an antigen-binding domain that specifically binds to glucocorticoid-induced TNF receptor (GITR). The provided antigen-binding domain includes a heavy chain variable region (VH) and light chain variable region (VL), where the VL comprises an amino acid sequence at least 80%, 85%, 90%, 95%, or 100% identical to SEQ ID NO: 5, SEQ ID NO: 13, SEQ ID NO: 21, SEQ ID NO: 29, SEQ ID NO: 37, SEQ ID NO: 45, SEQ ID NO: 53, SEQ ID NO: 61, SEQ ID NO: 69, SEQ ID NO: 77, SEQ ID NO: 85, SEQ ID NO: 93, SEQ ID NO: 101, SEQ ID NO: 109, SEQ ID NO: 117, SEQ ID NO: 125, SEQ ID NO: 133, SEQ ID NO: 141, SEQ ID NO: 149, SEQ ID NO: 157, SEQ ID NO: 165, SEQ ID NO: 173, SEQ ID NO: 181, SEQ ID NO: 189, SEQ ID NO: 207, SEQ ID NO: 215, SEQ ID NO: 223, SEQ ID NO: 231, SEQ ID NO: 239, SEQ ID NO: 247, SEQ ID NO: 255, SEQ ID NO: 263, SEQ ID NO: 271, SEQ ID NO: 279, SEQ ID NO: 287, SEQ ID NO: 295, SEQ ID NO: 303, SEQ ID NO: 311, SEQ ID NO: 319, SEQ ID NO: 327, SEQ ID NO: 335, SEQ ID NO: 343, SEQ ID NO: 351, SEQ ID NO: 359, SEQ ID NO: 367, SEQ ID NO: 375, SEQ ID NO: 383, or SEQ ID NO: 391. In certain aspects the VH comprises an amino acid sequence at least 80%, 85%, 90%, 95%, or 100% identical to SEQ ID NO: 1, SEQ ID NO: 9, SEQ ID NO: 17, SEQ ID NO: 25, SEQ ID NO: 33, SEQ ID NO: 41, SEQ ID NO: 49, SEQ ID NO: 57, SEQ ID NO: 65, SEQ ID NO: 73, SEQ ID NO: 81, SEQ ID NO: 89, SEQ ID NO: 97, SEQ ID NO: 105, SEQ ID NO: 113, SEQ ID NO: 121, SEQ ID NO: 129, SEQ ID NO: 137, SEQ ID NO: 145, SEQ ID NO: 153, SEQ ID NO: 161, SEQ ID NO: 169, SEQ ID NO: 177, SEQ ID NO: 185, SEQ ID NO: 203, SEQ ID NO: 211, SEQ ID NO: 219, SEQ ID NO: 227, SEQ ID NO: 235, SEQ ID NO: 243, SEQ ID NO: 251, SEQ ID NO: 259, SEQ ID NO: 267, SEQ ID NO: 275, SEQ ID NO: 283, SEQ ID NO: 291, SEQ ID NO: 299, SEQ ID NO: 307, SEQ ID NO: 315, SEQ ID NO: 323, SEQ ID NO: 331, SEQ ID NO: 339, SEQ ID NO: 347, SEQ ID NO: 355, SEQ ID NO: 363, SEQ ID NO: 371, SEQ ID NO: 379, or SEQ ID NO: 387. In certain aspects, the provided antigen binding domain can be included in an antibody or antigen-binding fragment, variant, or derivative thereof. In certain aspects the antibody is a multimeric, e.g., a dimeric pentameric, or hexameric anti-GITR antibody as described elsewhere herein.

This disclosure also provides an antigen-binding domain that specifically binds to glucocorticoid-induced TNF receptor (GITR). The provided antigen-binding domain includes a heavy chain variable region (VH) and light chain variable region (VL), where the wherein the VH and VL comprise, respectively, amino acid sequences at least 80%, at least 85%, at least 90%, at least 95% or 100% identical to the mature VH and VL amino acid sequences comprising, respectively, SEQ ID NO: 1 and SEQ ID NO: 5, SEQ ID NO: 9 and SEQ ID NO: 13, SEQ ID NO: 17 and SEQ ID NO: 21, SEQ ID NO: 25 and SEQ ID NO: 29, SEQ ID NO: 33 and SEQ ID NO: 37, SEQ ID NO: 41 and SEQ ID NO: 45, SEQ ID NO: 49 and SEQ ID NO: 53, SEQ ID NO: 57 and SEQ ID NO: 61, SEQ ID NO: 65 and SEQ ID NO: 69, SEQ ID NO: 73 and SEQ ID NO: 77, SEQ ID NO: 81 and SEQ ID NO: 85, SEQ ID NO: 89 and SEQ ID NO: 93, SEQ ID NO: 97 and SEQ ID NO: 101, SEQ ID NO: 105 and SEQ ID NO: 109, SEQ ID NO: 113 and SEQ ID NO: 117, SEQ ID NO: 121 and SEQ ID NO: 125, SEQ ID NO: 129 and SEQ ID NO: 133, SEQ ID NO: 137 and SEQ ID NO: 141, SEQ ID NO: 145 and SEQ ID NO: 149, SEQ ID NO: 153 and SEQ ID NO: 157, SEQ ID NO: 161 and SEQ ID NO: 165, SEQ ID NO: 169 and SEQ ID NO: 173, SEQ ID NO: 177 and SEQ ID NO: 181, SEQ ID NO: 185 and SEQ ID NO: 189, SEQ ID NO: 203 and SEQ ID NO: 207, SEQ ID NO: 211 and SEQ ID NO: 215, SEQ ID NO: 219 and SEQ ID NO: 223, SEQ ID NO: 227 and SEQ ID NO: 231, SEQ ID NO: 235 and SEQ ID NO: 239, SEQ ID NO: 243 and SEQ ID NO: 247, SEQ ID NO: 251 and SEQ ID NO: 255, SEQ ID NO: 259 and SEQ ID NO: 263, SEQ ID NO: 267 and SEQ ID NO: 271, SEQ ID NO: 275 and SEQ ID NO: 279, SEQ ID NO: 283 and SEQ ID NO: 287, SEQ ID NO: 291 and SEQ ID NO: 295, SEQ ID NO: 299 and SEQ ID NO: 303, SEQ ID NO: 307 and SEQ ID NO: 311, SEQ ID NO: 315 and SEQ ID NO: 319, SEQ ID NO: 323 and SEQ ID NO: 327, SEQ ID NO: 331 and SEQ ID NO: 335, SEQ ID NO: 339 and SEQ ID NO: 343, SEQ ID NO: 347 and SEQ ID NO: 351, SEQ ID NO: 355 and SEQ ID NO: 359, SEQ ID NO: 363 and SEQ ID NO: 367, SEQ ID NO: 371 and SEQ ID NO: 375, SEQ ID NO: 379 and SEQ ID NO: 383, or SEQ ID NO: 387 and SEQ ID NO: 391. In certain aspects, the provided antigen binding domains can be included in an antibody or antigen-binding fragment, variant, or derivative thereof. In certain aspects the antibody is a multimeric, e.g., a dimeric pentameric, or hexameric anti-GITR antibody as described elsewhere herein.

In certain aspects the antigen-binding domain as provided above is an Fv fragment, e.g., a single-chain Fv fragment (scFv), or a disulfide-linked Fv fragment (sdFv).

In certain aspects the antigen-binding domain as provided above is included in an antibody or fragment or derivative thereof as described elsewhere herein.

In certain aspects the antibody or fragment or derivative thereof comprises a single bivalent binding unit comprising two antigen-binding domains wherein at least one antigen-binding domain specifically binds to GITR. According to this aspect, the binding unit comprises two heavy chains each comprising a heavy chain constant region or fragment or variant thereof, and wherein at least one heavy chain constant region or variant thereof of the binding unit is fused to a copy of the provided VH of the antigen-binding domain. In certain aspects, both heavy chain constant regions or fragments or variants thereof of the binding unit are fused to a copy of the provided VH of the antigen-binding domain. In certain aspects, the heavy chains comprise IgG heavy chain constant regions or fragments or variants thereof. IgG heavy chain constant regions and fragments thereof are described elsewhere herein and are well-known by persons of skill in the art. In certain aspects the single bivalent binding unit further comprises two light chains each comprising a light chain constant region or fragment or variant thereof. In certain aspects at least one light chain constant region is fused to a copy of the provided VL of the antigen-binding domain. In certain aspects both light chain constant regions or fragments or variants thereof of the binding unit are fused to a copy of the provided VL of the antigen-binding domain. In certain aspects, the single bivalent binding unit comprises a complete antibody, e.g., a complete IgG antibody, a Fab fragment, a Fab′ fragment, or an F(ab′)2 fragment. In certain aspects, the single bivalent binding unit is a human antibody, fragment, or derivative thereof.

In certain aspects, the provided antigen-binding domain is included in a multimeric antibody or fragment or derivative thereof comprising two, five, or six bivalent binding units, where the antibody comprises four, ten, or twelve antigen-binding domains. In certain aspects at least one, two, three, four, five, six, seven, eight, nine, ten, eleven, or twelve of the antigen-binding domains specifically binds to GITR. As provided herein, at least one, two, three, four, five, six, seven, eight, nine, ten, eleven, or twelve of the antigen-binding domains comprise the VH and VL amino acid sequences as provided above. According to these aspects, each binding unit comprises two heavy chains each comprising an IgA or IgM constant region or a multimerizing fragment or variant thereof, and at least one of the heavy chain constant regions of the binding unit is fused to a copy of the provided VH of the provided antigen-binding domain. In certain aspects the antibody or fragment or derivative thereof is a human antibody, fragment, or derivative thereof.

In certain aspects, the provided antibody or fragment or derivative thereof is dimeric and comprises two bivalent IgA binding units and a J chain or fragment or variant thereof, where each binding unit comprises two IgA heavy chain constant regions or multimerizing fragments or variants thereof. In certain aspects the dimeric antibody or fragment or derivative thereof can further comprise a secretory component, or fragment or variant thereof. In certain aspects, the IgA heavy chain constant regions or fragments or variants thereof each comprise a Cα3-tp domain, and can further comprise a Cα1 domain, a Cα2 domain, an IgA hinge region, or any combination thereof.

In certain aspects, the provided antibody or fragment or derivative thereof is hexameric or pentameric and comprises five or six bivalent IgM binding units, wherein each binding unit comprises two IgM heavy chain constant regions or multimerizing fragments or variants thereof. In certain aspects the IgM heavy chain constant regions or fragments or variants thereof each comprise a Cμ4-tp domain or fragment or variant thereof, and can further comprise a Cλ1 domain, a Cμ2 domain, a Cμ3 domain, or any combination thereof. In certain aspects the antibody or fragment or derivative thereof is pentameric, and further comprises a J chain, or fragment thereof, or variant thereof. In certain aspects, each binding unit further comprises two light chains each comprising a light chain constant region or fragment or variant thereof, and wherein at least one, two, three, four, five, six, seven eight, nine, ten, eleven, or twelve light chain constant regions are fused to a copy of the provided VL of the antigen-binding domain. In certain aspects the antibody or fragment or derivative thereof is a human antibody, fragment, or derivative thereof.

The antibody or fragment or derivative thereof as provided herein can, in certain aspects, be multispecific.

In certain aspects the provided antigen-binding domain, or an antibody or fragment or derivative comprising the antigen binding domain can specifically bind to human GITR, mouse GITR, non-human primate GITR, or any combination thereof. In certain aspects the non-human primate GITR is cynomolgus monkey GITR. In certain aspects the provided antigen-binding domain, or an antibody or fragment or derivative comprising the antigen binding domain binds to GITR with an affinity characterized by a dissociation constant KD no greater than 500 nM, 100 nM, 50.0 nM, 40.0 nM, 30.0 nM, 20.0 nM, 10.0 nM, 9.0 nM, 8.0 nM, 7.0 nM, 6.0 nM, 5.0 nM, 4.0 nM, 3.0 nM, 2.0 nM, 1.0 nM, 0.50 nM, 0.10 nM, 0.050 nM, 0.01 nM, 0.005 nM, or 0.001 nM; and wherein the GITR is human GITR, mouse GITR, cynomolgus monkey GITR, or any combination thereof.

IgM or IgM-Like antibodies

IgM is the first immunoglobulin produced by B cells in response to stimulation by antigen and is naturally present at around 1.5 mg/ml in serum with a half-life of about 5 days. IgM is typically a pentameric or hexameric molecule. An IgM binding unit includes two light and two heavy chains. While IgG contains three heavy chain constant domains (CH1, CH2 and CH3), the heavy (μ) chain of IgM additionally contains a fourth constant domain (CH4), that includes a C-terminal “tailpiece.” The human IgM constant region typically comprises the amino acid sequence SEQ ID NO: 193. The human Cμ1 region ranges from about amino acid 5 to about amino acid 102 of SEQ ID NO: 193; the human Cμ2 region ranges from about amino acid 114 to about amino acid 205 of SEQ ID NO: 193, the human Cμ3 region ranges from about amino acid 224 to about amino acid 319 of SEQ ID NO: 193, the Cμ4 region ranges from about amino acid 329 to about amino acid 430 of SEQ ID NO: 193, and the tailpiece ranges from about amino acid 431 to about amino acid 453 of SEQ ID NO: 193. SEQ ID NO: 193 is presented below.

SEQ ID NO: 193:
GSASAPTLFPLVSCENSPSDTSSVAVGCLAQDFLPD
SITLSWKYKNNSDISSTRGFPSVLRGGKYAATSQV
LLPSKDVMQGTDEHVVCKVQHPNGNKEKNVPLPVI
AELPPKVSVFVPPRDGFFGNPRKSKLICQATGFSP
RQIQVSWLREGKQVGSGVTTDQVQAEAKESGPTTY
KVTSTLTIKESDWLGQSMFTCRVDHRGLTFQQNAS
SMCVPDQDTAIRVFAIPPSFASIFLTKSTKLTCLV
TDLTTYDSVTISWTRQNGEAVKTHTNISESHPNAT
FSAVGEASICEDDWNSGERFTCTVTHTDLPSPLKQ
TISRPKGVALHRPDVYLLPPAREQLNLRESATITC
LVTGFSPADVFVQWMQRGQPLSPEKYVTSAPMPEP
QAPGRYFAHSILTVSEEEWNTGETYTCVAHEALPN
RVTERTVDKSTGKPTLYNVSLVMSDTAGTCY

Five IgM binding units can form a complex with an additional small polypeptide chain (the J-chain) to form an IgM antibody. The precursor human J-chain comprises the amino acid sequence SEQ ID NO: 194. SEQ ID NO: 1 is presented below.

SEQ ID NO: 194:
MKNHLLFWGVLAVFIKAVHVKAQEDERIVLVDNKCKCARI
TSRIIRSSEDPNEDIVERNIRIIVPLNNRENISDPTSPLR
TRFVYHLSDLCKKCDPTEVELDNQIVTATQSNICDEDSAT
ETCYTYDRNKCYTAVVPLVYGGETKMVETALTPDACYPD

The mature human J-chain comprises the amino acid sequence SEQ ID NO: 195. Without the J-chain, IgM binding units typically assemble into a hexamer. While not wishing to be bound by theory, the assembly of IgM binding units into a pentameric or hexameric binding molecule is thought to involve the Cμ3 and Cμ4 domains. Accordingly, a pentameric or hexameric binding molecule provided in this disclosure typically includes IgM constant regions that include at least the Cμ3 and Cμ4 domains. SEQ ID NO: 195 is presented below.

SEQ ID NO: 195:
QEDERIVLVDNKCKCARITSRIIRSSEDPNEDIVE
RNIRIIVPLNNRENISDPTSPLRTRFVYHLSDLCK
KCDPTEVELDNQIVTATQSNICDEDSATETCYTYD
RNKCYTAVVPLVYGGETKMVETALTPDACYPD

An IgM heavy chain constant region can additionally include a Cμ2 domain or a fragment thereof, a Cμ1 domain or a fragment thereof, and/or other IgM heavy chain domains. In certain aspects, a binding molecule, e.g., an antibody or fragment, variant, or derivative thereof as provided herein can include a complete IgM heavy (μ) chain constant domain, e.g., SEQ ID NO: 193, or a variant, derivative, or analog thereof.

In certain aspects, the disclosure provides a pentameric IgM or IgM-like antibody comprising five bivalent binding units, respectively, where each binding unit includes two IgM heavy chain constant regions or fragments or variants thereof. In certain aspects, the two IgM heavy chain constant regions are human heavy chain constant regions.

Where the IgM or IgM-like antibody provided herein is pentameric, the IgM or IgM-like antibody further comprises a J-chain, or functional fragment thereof, or variant thereof. In certain aspects the J-chain can be modified or mutated to affect serum half-life of the IgM or IgM-like antibody provided herein, as discussed elsewhere herein.

An IgM heavy chain constant region can include one or more of a Cμ1 domain or fragment or variant thereof, a Cμ2 domain or fragment or variant thereof, a Cμ3 domain or fragment or variant thereof, and/or a Cμ4 domain or fragment or variant thereof, provided that the constant region can serve a desired function in the an IgM or IgM-like antibody, e.g., associate with second IgM constant region to form a binding domain, or associate with other binding units to form a hexamer or a pentamer. In certain aspects the two IgM heavy chain constant regions or fragments or variants thereof within an individual binding unit each comprise a Cμ3 domain or fragment or variant thereof, a Cμ4 domain or fragment or variant thereof, a tailpiece (TP) or fragment or variant thereof, or any combination of a Cμ3 domain a Cu domain, and a TP or fragment or variant thereof. In certain aspects the two IgM heavy chain constant regions or fragments or variants thereof within an individual binding unit each further comprise a Cμ2 domain or fragment or variant thereof, a Cμ1 domain or fragment or variant thereof, or a Cμ1 domain or fragment or variant thereof and a Cμ2 domain or fragment or variant thereof.

### Agonistic Pentameric or Hexameric GITR Antibodies

This disclosure provides a pentameric or hexameric antibody, e.g., an antibody, or fragment, variant, or derivative thereof with five or six “binding units” as defined herein, that can specifically bind to three or more, e.g., four or more, e.g., five, six, seven, eight, nine, ten, eleven, or twelve GITR monomers, e.g., murine, non-human primate, and/or human GITR monomers. In certain aspects, where GITR is expressed on a cell, e.g., a T cell, e.g., a Treg or an activated effector CTL, a pentameric or hexameric antibody or multimerizing fragment, variant, or derivative thereof as provided herein can sufficiently engage multiple, e.g., three or more GITR monomers on the cell to trigger a signal transduction pathway in the absence of a secondary cross-linking moiety, thereby inducing anti-tumor immunity. A pentameric or hexameric antibody or multimerizing fragment, variant, or derivative thereof as provided herein can possess improved binding characteristics or biological activity as compared to a binding molecule composed of a single binding unit, e.g., a bivalent IgG antibody. For example, a pentameric or hexameric antibody or multimerizing fragment, variant, or derivative thereof can more efficiently cross-link multiple, e.g., three or more GITR receptors on the surface of a cell, and/or can effectively cross-link multiple, e.g., three or more GITR receptors on the surface of a cell in the absence of a secondary cross-linking moiety such as, but not limited to an FcγR, thereby facilitating anti-tumor immunity.

A pentameric or hexameric antibody or multimerizing fragment, variant, or derivative thereof as provided herein can likewise possess distinctive characteristics compared to multivalent binding molecules composed of synthetic or chimeric structures. For example, use of human IgM constant regions can afford reduced immunogenicity and thus increased safety relative to a binding molecule containing chimeric constant regions or synthetic structures. Moreover, an IgM-based antibody can consistently form hexameric or pentameric oligomers resulting in a more homogeneous expression product. Superior complement fixation can also be an advantageous effector function of IgM-based antibodies.

In certain aspects, the disclosure provides a pentameric or hexameric antibody or multimerizing fragment, variant, or derivative thereof comprising five or six bivalent binding units, respectively, where each binding unit includes two IgM heavy chain constant regions or multimerizing fragments or variants or derivatives thereof. In certain aspects, the two IgM heavy chain constant regions are human heavy chain constant regions.

Where the binding molecule provided herein is pentameric, the binding molecule can further comprise a J chain, or fragment thereof, or variant thereof. In certain aspects the J chain can be modified, as discussed elsewhere herein.

An IgM heavy chain constant region can include one or more of a Cμ1 domain or fragment or variant thereof, a Cμ2 domain or fragment or variant thereof, a Cμ3 domain or fragment or variant thereof, and/or a Cμ4-tp domain or fragment or variant thereof, provided that the constant region can serve a desired function in the binding molecule, e.g., associate with second IgM constant region to form a binding domain, or associate with other binding units to form a hexamer or a pentamer. In certain aspects the two IgM heavy chain constant regions or fragments or variants thereof within an individual binding unit each comprise a Cμ4 domain or fragment or variant thereof and a tailpiece (TP) or fragment or variant thereof. In certain aspects the two IgM heavy chain constant regions or fragments or variants thereof within an individual binding unit each further comprise a Cμ3 domain or fragment or variant thereof, a Cμ2 domain or fragment or variant thereof, a Cμ1 domain or fragment or variant thereof, or a Cμ1 domain or fragment or variant thereof and a Cμ2 domain or fragment or variant thereof, and a Cμ3 domain or fragment or variant thereof.

In certain aspects each of the two IgM heavy chain constant regions in a given binding unit is associated with an antigen-binding domain, for example an Fv portion of an antibody, e.g., a VH and a VL of a human or murine antibody, where the VL can be associated with a light chain constant region. In a hexameric or pentameric antibody or multimerizing fragment, variant, or derivative thereof as provided herein at least three antigen-binding domains of the binding molecule are GITR binding domains that can specifically and agonistically bind to GITR, e.g., human, non-human primate, and/or murine GITR.

### IgA and IgA-Like Antibodies

IgA plays a critical role in mucosal immunity and comprises about 15% of total immunoglobulin produced. IgA is a monomeric or dimeric molecule. An IgA binding unit includes two light and two heavy chains. IgA contains three heavy chain constant domains (Cα1, Cα2 and Cα3), and includes a C-terminal “tailpiece.” Human IgA has two subtypes, IgA1 and IgA2. The human IgA1 constant region typically comprises the amino acid sequence SEQ ID NO: 198. The human Cα1 region ranges from about amino acid 6 to about amino acid 98 of SEQ ID NO: 198; the human Cα2 region ranges from about amino acid 125 to about amino acid 220 of SEQ ID NO: 198, the human Cα3 region ranges from about amino acid 228 to about amino acid 330 of SEQ ID NO: 198, and the tailpiece ranges from about amino acid 331 to about amino acid 352 of SEQ ID NO: 198. The human IgA2 constant region typically comprises the amino acid sequence SEQ ID NO: 199. The human Cα1 region ranges from about amino acid 6 to about amino acid 98 of SEQ ID NO: 199; the human Cα2 region ranges from about amino acid 112 to about amino acid 207 of SEQ ID NO: 199, the human Cα3 region ranges from about amino acid 215 to about amino acid 317 of SEQ ID NO: 199, and the tailpiece ranges from about amino acid 318 to about amino acid 340 of SEQ ID NO: 199. SEQ ID NOS: 3 and 4 are presented below:

SEQ ID NO: 198
ASPTSPKVFPLSLCSTQPDGNVVIACLVQGFFPQE
PLSVTWSESGQGVTARNFPPSQDASGDLYTTSSQL
TLPATQCLAGKSVTCHVKHYTNPSQDVTVPCPVPS
TPPTPSPSTPPTPSPSCCHPRLSLHRPALEDLLLG
SEANLTCTLTGLRDASGVTFTWTPSSGKSAVQGPP
ERDLCGCYSVSSVLPGCAEPWNHGKTFTCTAAYPE
SKTPLTATLSKSGNTFRPEVHLLPPPSEELALNEL
VTLTCLARGFSPKDVLVRWLQGSQELPREKYLTWA
SRQEPSQGTTTFAVTSILRVAAEDWKKGDTFSCMV
GHEALPLAFTQKTIDRLAGKPTHVNVSVVMAEVDG
TCY
SEQ ID NO: 199
ASPTSPKVFPLSLDSTPQDGNVVVACLVQGFFPQE
PLSVTWSESGQNVTARNFPPSQDASGDLYTTSSQL
TLPATQCPDGKSVTCHVKHYTNPSQDVTVPCPVPP
PPPCCHPRLSLHRPALEDLLLGSEANLTCTLTGLR
DASGATFTWTPSSGKSAVQGPPERDLCGCYSVSSV
LPGCAQPWNHGETFTCTAAHPELKTPLTANITKSG
NTFRPEVHLLPPPSEELALNELVTLTCLARGFSPK
DVLVRWLQGSQELPREKYLTWASRQEPSQGTTTFA
VTSILRVAAEDWKKGDTFSCMVGHEALPLAFTQKT
IDRMAGKPTHVNVSVVMAEVDGTCY

Two IgA binding units can form a complex with two additional polypeptide chains, the J chain (SEQ ID NO: 195) and the secretory component (precursor, SEQ ID NO: 200, mature, SEQ ID NO: 201) to form a secretory IgA (sIgA) antibody. While not wishing to be bound by theory, the assembly of IgA binding units into a dimeric sIgA binding molecule is thought to involve the Cα3 and tailpiece domains. Accordingly, a dimeric sIgA binding molecule provided in this disclosure typically includes IgA constant regions that include at least the Cα3 and tailpiece domains. SEQ ID NO: 200 and SEQ ID NO: 201 are presented below:

SEQ ID NO: 200:
MLLFVLTCLLAVFPAISTKSPIFGPEEVNSVEGNS
VSITCYYPPTSVNRHTRKYWCRQGARGGCITLISS
EGYVSSKYAGRANLTNFPENGTFVVNIAQLSQDDS
GRYKCGLGINSRGLSFDVSLEVSQGPGLLNDTKVY
TVDLGRTVTINCPFKTENAQKRKSLYKQIGLYPVL
VIDSSGYVNPNYTGRIRLDIQGTGQLLFSVVINQL
RLSDAGQYLCQAGDDSNSNKKNADLQVLKPEPELV
YEDLRGSVTFHCALGPEVANVAKFLCRQSSGENCD
VVVNTLGKRAPAFEGRILLNPQDKDGSFSVVITGL
RKEDAGRYLCGAHSDGQLQEGSPIQAWQLFVNEES
TIPRSPTVVKGVAGGSVAVLCPYNRKESKSIKYWC
LWEGAQNGRCPLLVDSEGWVKAQYEGRLSLLEEPG
NGTFTVILNQLTSRDAGFYWCLTNGDTLWRTTVEI
KIIEGEPNLKVPGNVTAVLGETLKVPCHFPCKFSS
YEKYWCKWNNTGCQALPSQDEGPSKAFVNCDENSR
LVSLTLNLVTRADEGWYWCGVKQGHFYGETAAVYV
AVEERKAAGSRDVSLAKADAAPDEKVLDSGFREIE
NKAIQDPRLFAEEKAVADTRDQADGSRASVDSGSS
EEQGGSSRALVSTLVPLGLVLAVGAVAVGVARARH
RKNVDRVSIRSYRTDISMSDFENSREFGANDNMGA
SSITQETSLGGKEEFVATTESTTETKEPKKAKRSS
KEEAEMAYKDFLLQSSTVAAEAQDGPQEA
SEQ ID NO: 201:
KSPIFGPEEVNSVEGNSVSITCYYPPTSVNRHTRK
YWCRQGARGGCITLISSEGYVSSKYAGRANLTNFP
ENGTFVVNIAQLSQDDSGRYKCGLGINSRGLSFDV
SLEVSQGPGLLNDTKVYTVDLGRTVTINCPFKTEN
AQKRKSLYKQIGLYPVLVIDSSGYVNPNYTGRIRL
DIQGTGQLLFSVVINQLRLSDAGQYLCQAGDDSNS
NKKNADLQVLKPEPELVYEDLRGSVTFHCALGPEV
ANVAKFLCRQSSGENCDVVVNTLGKRAPAFEGRIL
LNPQDKDGSFSVVITGLRKEDAGRYLCGAHSDGQL
QEGSPIQAWQLFVNEESTIPRSPTVVKGVAGGSVA
VLCPYNRKESKSIKYWCLWEGAQNGRCPLLVDSEG
WVKAQYEGRLSLLEEPGNGTFTVILNQLTSRDAGF
YWCLTNGDTLWRTTVEIKIIEGEPNLKVPGNVTAV
LGETLKVPCHFPCKFSSYEKYWCKWNNTGCQALPS
QDEGPSKAFVNCDENSRLVSLTLNLVTRADEGWYW
CGVKQGHFYGETAAVYVAVEERKAAGSRDVSLAKA
DAAPDEKVLDSGFREIENKAIQDPR

An IgA heavy chain constant region can additionally include a Cα2 domain or a fragment thereof, a Cα1 domain or a fragment thereof, and/or other IgA heavy chain domains. In certain aspects, a binding molecule as provided herein can include a complete IgA heavy (a) chain constant domain (e.g., SEQ ID NO: 198 or SEQ ID NO: 199), or a variant, derivative, or analog thereof.

### Agonistic Dimeric GITR Antibodies

This disclosure provides a dimeric antibody, e.g., an antibody, or fragment, variant, or derivative thereof with two IgA “binding units” as defined herein that can specifically bind to three or more or up to four GITR monomers, e.g., human, non-human primate, or murine GITR monomers. In certain aspects, where GITR is expressed on a cell, e.g., a T cell, e.g., a Treg or an activated effector CTL, contacting multiple GITR receptors on the cell with a dimeric antibody or multimerizing fragment, variant, or derivative thereof as provided herein can trigger a signal transduction pathway in the absence of a secondary cross-linking moiety, thereby inducing anti-tumor immunity. A dimeric antibody or multimerizing fragment, variant, or derivative thereof as provided herein can possess improved binding characteristics or biological activity as compared to a binding molecule composed of a single binding unit, e.g., a bivalent IgG antibody. For example, a dimeric antibody or multimerizing fragment, variant, or derivative thereof can more efficiently cross-link multiple, e.g., three or more GITR receptors on the surface of a cell, and/or can effectively cross-link multiple, e.g., three or more GITR receptors on the surface of a cell in the absence of a secondary cross-linking moiety such as, but not limited to a FcγR, thereby facilitating anti-tumor immunity. Moreover, a dimeric antibody or multimerizing fragment, variant, or derivative thereof can reach mucosal sites providing greater tissue distribution for the binding molecules provided herein. Use of an IgA-based dimeric antibody or multimerizing fragment, variant, or derivative thereof can allow, for example, greater tissue distribution for an antibody as provided herein. Mucosal distribution could be beneficial to reach the tumor microenvironment of certain cancers, e.g., lung cancer, ovarian cancer, colorectal cancer, or squamous cell carcinoma. Likewise, a dimeric antibody or multimerizing fragment, variant, or derivative thereof as provided herein can possess binding characteristics or biological activity that can be distinguished from an antibody comprising five or six binding units, e.g., a hexameric or pentameric IgM antibody. For example, a dimeric antibody or multimerizing fragment, variant, or derivative thereof would be smaller, and could, for example, achieve better tissue penetration in certain solid tumors.

In certain aspects, the disclosure provides a dimeric antibody or multimerizing fragment, variant, or derivative thereof comprising two bivalent binding units, where each binding unit includes two IgA heavy chain constant regions or multimerizing fragments or variants thereof. In certain aspects, the two IgA heavy chain constant regions are human heavy chain constant regions.

A dimeric IgA antibody or multimerizing fragment, variant, or derivative thereof as provided herein can further comprise a J chain, or fragment thereof, or variant thereof, e.g., a modified J chain as disclosed elsewhere herein. A dimeric IgA antibody or multimerizing fragment, variant, or derivative thereof as provided herein can further comprise a secretory component, or fragment thereof, or variant thereof.

An IgA heavy chain constant region can include one or more of a Cα1 domain, a Cα2 domain, and/or a Cα3 domain, provided that the constant region can serve a desired function in the antibody, e.g., associate with a light chain constant region to facilitate formation of an antigen-binding domain, or associate with another IgA binding unit to form a dimeric antibody or multimerizing fragment, variant, or derivative thereof. In certain aspects the two IgA heavy chain constant regions or multimerizing fragments or variants thereof within an individual binding unit each comprise a Cα3 domain or fragment or variant thereof, a tailpiece (TP) or fragment or variant thereof, or any combination of a Cα3 domain, a TP, or fragment or variant thereof. In certain aspects the two IgA heavy chain constant regions or multimerizing fragments thereof within an individual binding unit each further comprise a Cα2 domain or fragment or variant thereof, a Cα1 domain or fragment or variant thereof, or a Cα1 domain or fragment or variant thereof and a Cα2 domain or fragment or variant thereof.

In certain aspects each of the two IgA heavy chain constant regions in a given binding unit is associated with an antigen binding domain, for example an Fv portion of an antibody, e.g., a VH and a VL of a human or murine antibody, where the VL can be associated with a light chain constant region. In a binding molecule as provided herein at least three antigen-binding domains of the binding molecule specifically and agonistically bind to GITR, e.g., human and/or murine GITR.

### Multispecific Dimeric, Pentameric, or Hexameric GITR Agonist Antibodies

A multi-specific, e.g., bispecific dimeric GITR agonist antibody or fragment, variant, or derivative thereof as provided herein can be based on the dimeric form of an IgA antibody, in which two pairs of IgA heavy chain sequences can be present with or without associated light chain sequences. For example, a bispecific dimeric GITR agonist antibody or fragment, variant, or derivative thereof as provided herein can be composed of two IgA (IgA1 or IgA2) dimers, including a J chain, e.g., a modified J chain as provided elsewhere herein.

A multi-specific, e.g., bispecific dimeric GITR agonist antibody or fragment, variant, or derivative thereof as provided herein can include mono- and bispecific binding units as long as the molecule as a whole has at least two binding specificities, e.g., at least two non-identical antigen-binding domains, e.g., different epitopes of GITR, epitopes from other TNFSFR molecules, or heterologous antigens.

Thus, in one embodiment, a multi-specific, e.g., bispecific dimeric antibody or fragment, variant, or derivative thereof as provided herein can include two monospecific binding units (AA, BB), each having bivalent binding specificity to a different binding target. In another embodiment, a multi-specific, e.g., bispecific dimeric antibody or fragment, variant, or derivative thereof as provided herein can include two bispecific binding units, each binding unit binding to the same two binding targets (AB, AB) to form a bispecific dimeric binding molecule. In a further embodiment, one binding unit present in a multi-specific dimeric antibody or fragment, variant, or derivative thereof as provided herein is monospecific (AA) while the other binding units are bispecific (BC), resulting in a multispecific binding molecule with three (A, B, C) binding specificities. In a further embodiment, each binding unit is bispecific, but one specificity is overlapping (e.g. AB, AC), resulting in a multispecific binding molecule with three (A, B, C) binding specificities. Other combinations, e.g., with four non-identical antigen binding domains (A, B, C, D) can be readily made based on this disclosure.

A multi-specific, e.g., bispecific pentameric or hexameric GITR agonist antibody or fragment, variant, or derivative thereof as provided herein can be based on the pentameric or hexameric forms of an IgM or IgM-like antibody, in which five or six pairs of IgM heavy chain sequences can be present with or without associated light chain sequences. For example, a bispecific hexameric or pentameric GITR agonist antibody or fragment, variant, or derivative thereof as provided herein can be composed of five IgM dimers, including a J chain, e.g., a modified J chain as provided elsewhere herein, or six IgM dimers.

A multi-specific, e.g., bispecific pentameric or hexameric GITR agonist antibody or fragment, variant, or derivative thereof as provided herein can include mono- and/or bispecific binding units as long as the molecule as a whole has at least two binding specificities, e.g., at least two non-identical antigen-binding domains, e.g., different epitopes of GITR, epitopes from other TNFSFR molecules, or heterologous antigens.

As discussed above for multispecific dimeric antibodies, each of the five or six binding units can independently be monospecific or bispecific (e.g., AA, BB, CC, etc.) or one or more binding units can be bispecific (e.g., AB, AB, AC, CD, etc.). Thus, a multi-specific, e.g., bispecific pentameric or hexameric antibody or fragment, variant, or derivative thereof as provided herein can include at least two independent antigen binding domains, and up to twelve different, independent antigen binding domains.

### Modified J Chains

In certain aspects, the J chain of a dimeric or pentameric antibody or fragment or derivative thereof as provided herein can be modified, e.g., by introduction of a heterologous moiety, or two or more heterologous moieties, without interfering with the ability of the IgM or IgA antibody to assemble and bind to its binding target(s). See U.S. Pat. No. 9,951,134, PCT Application No. PCT/US2016/055053 (Publication WO 2017/059387), PCT Application No. PCT/US2016/055041 (Publication WO 2017/059380), and PCT Appl. No. PCT/US2019/20374, each of which is incorporated herein by reference in its entirety. Accordingly, dimeric or pentameric antibodies or multimerizing fragments or derivatives thereof as provided herein, including multispecific dimeric or pentameric antibodies or multimerizing fragments or derivatives thereof as described elsewhere herein, can comprise a modified J chain or functional fragment thereof comprising a heterologous moiety introduced into the J chain or fragment thereof. In certain aspects heterologous moiety can be a peptide or polypeptide sequence fused in frame to the J chain or chemically conjugated to the J chain. In certain aspects the heterologous moiety can be a chemical moiety conjugated to the J chain. Heterologous moieties to be attached to a J chain can include, without limitation, a binding moiety, e.g., an antibody or antigen binding fragment thereof, e.g., a single chain Fv (ScFv) molecule, a stabilizing peptide that can increase the half-life of the dimeric or pentameric binding molecule, or a chemical moiety such as a polymer or a cytotoxin.

In some aspects, a modified J chain can comprise an antigen binding domain that can include, without limitation, a polypeptide (including small peptides) capable of specifically binding to a target antigen. In certain aspects, an antigen binding domain associated with a modified J chain can be an antibody or an antigen-binding fragment thereof, as described elsewhere herein. In certain aspects the antigen binding domain can be a scFv binding domain or a single-chain binding domain derived, e.g., from a camelid or condricthoid antibody. The antigen binding domain can be introduced into the J chain at any location that allows the binding of the antigen binding domain to its binding target without interfering with J chain function or the function of an associated IgM or IgA antibody. Insertion locations include but are not limited to: at or near the C-terminus, at or near the N-terminus or at an internal location that, based on the three-dimensional structure of the J chain, is accessible. In certain aspects, the antigen binding domain can be introduced into the human J chain of SEQ ID NO: 195 between cysteine residues 92 and 101 of SEQ ID NO: 195. In a further aspect, the antigen binding domain can be introduced into the human J chain of SEQ ID NO: 2 at or near a glycosylation site. In a further aspect, the antigen binding domain can be introduced into the human J chain of SEQ ID NO: 195 within about 10 amino acid residues from the C-terminus.

Pentameric IgM or IgM-Like Antibodies with J-Chain Mutations That Alter Serum Half-Life

In certain aspects an IgM antibody or multimerizing fragment thereof, or a pentameric

IgM-like antibody, or a multimerizing fragment thereof as provided herein comprises alterations that can enhance serum half-life. In certain aspects, such an IgM or IgM-like antibody comprises a functional variant and/or derivative of a J-chain or functional fragment thereof. By a “functional variant, derivative, or fragment” of a J-chain is meant a J-chain variant, derivative, or fragment that remains capable of associating with five IgM binding units to form a pentamer. As provided herein, the variant and/or derivative J-chain or functional fragment thereof can include one or more single amino acid substitutions, deletions, or insertions that can affect serum half-life of an antibody comprising the J-chain or functional fragment, variant, and/or derivative thereof. The term “one or more single amino acid substitutions, insertions, and deletions” means that each amino acid of the J-chain or functional fragment, variant, and/or derivative thereof amino acid sequence can individually be substituted, deleted, or can have a single amino acid inserted adjacent thereto, but the J-chain or functional fragment, variant, and/or derivative thereof must still be able to serve the function of assembling with IgM heavy chains or IgM-like heavy chains and antibody light chains to form an IgM pentamer or IgM-like pentamer. In certain aspects the J-chain or functional fragment, variant, and/or derivative thereof as provided herein can have a single amino acid substitution, insertion or deletion, a combination of two single amino acid substitutions, insertions, or deletions (e.g., two single amino acid substitutions or one single amino acid substitution and one single amino acid insertion or deletion), a combination of three single amino acid substitutions, insertions, or deletions, a combination of four single amino acid substitutions, insertions, or deletions or more, where the one, two, three, four, or more single amino acid substitutions, insertions or deletions can affect the serum half-life of an IgM antibody or IgM-like antibody comprising the J-chain or functional fragment, variant, and/or derivative thereof. Accordingly, the provided IgM or IgM-like antibody exhibits an increased serum half-life upon administration to an animal relative to a reference IgM or IgM-like antibody that is identical, except for the one or more single amino acid substitutions, deletions, or insertions in the J-chain or functional fragment, variant, and/or derivative thereof, where both the provided antibody and the reference IgM or IgM-like antibody are administered in the same way to the same animal species.

In certain aspects, the serum half-life of the IgM or IgM-like antibody, e.g., the α half-life, the β half-life, or the overall half-life, can be increased by at least 0.1-fold, at least 0.5-fold, at least 1-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, at least 500-fold, at least 1000-fold or more over the reference IgM or IgM-like antibody that is identical, except for the one or more single amino acid substitutions, deletions, or insertions in the J-chain or functional fragment, variant, and/or derivative thereof, where both the provided antibody and the reference IgM or IgM-like antibody are administered in the same way to the same animal species. In certain aspects, the increase in serum half-life approaches that of an IgG antibody comprising the same antigen-binding domains.

In certain aspects, the J-chain of the IgM antibody or IgM-like antibody as provided herein comprises an amino acid substitution at the amino acid position corresponding to amino acid Y102 of the mature human J-chain (SEQ ID NO: 195). By “an amino acid corresponding to amino acid Y102 of the wild-type human J-chain” is meant the amino acid in the sequence of the J-chain of any species which is homologous to Y102 in the human J-chain. The position corresponding to Y102 in SEQ ID NO: 195 is conserved in the J-chain amino acid sequences of at least 43 other species. See FIG. 4 of U.S. Pat. No. 9,951,134, which is incorporated by reference herein. In certain aspects, Y102 of SEQ ID NO: 195 can be substituted with any amino acid. In certain aspects, Y102 of SEQ ID NO: 195 can be substituted with alanine (A), serine (S) or arginine (R). In a particular aspect, Y102 of SEQ ID NO: 195 can be substituted with alanine. In a particular aspect the J-chain or functional fragment, variant, and/or derivative thereof is a variant human J-chain and comprises the amino acid sequence SEQ ID NO: 202. SEQ ID NO: 202 is presented below.

SEQ ID NO: 202:
QEDERIVLVDNKCKCARITSRIIRSSEDPNEDIV
ERNIRIIVPLNNRENISDPTSPLRTRFVYHLSDL
CKKCDPTEVELDNQIVTATQSNICDEDSATETCA
TYDRNKCYTAVVPLVYGGETKMVETALTPDACYP
D

Additional J-chain mutations affecting serum half-life, as well as IgM Fc mutations that affect serum half-life are disclosed in PCT Appl. No. PCT/US2019/20374, which is incorporated herein by reference in its entirety.

### Polynucleotides, Vectors, and Host Cells

The disclosure further provides a polynucleotide, e.g., an isolated, recombinant, and/or non-naturally-occurring polynucleotide, comprising a nucleic acid sequence that encodes an antigen-binding domain as provided herein or a polypeptide subunit of the dimeric, hexameric, or pentameric antibody or fragment or derivative thereof as provided herein. By “polypeptide subunit” is meant a portion of an antibody, binding unit, or antigen-binding domain that can be independently translated. Examples include, without limitation, an antibody variable domain, e.g., a VH or a VL, a J chain, a secretory component, a single chain Fv, an antibody heavy chain, an antibody light chain, an antibody heavy chain constant region, an antibody light chain constant region, and/or any fragment, variant, or derivative thereof.

To form the antigen-binding domains or the variable regions of antibodies that specifically bind to GITR, the provided polynucleotides can be inserted into expression vector templates, e.g., for a monomeric antibody, e.g., an IgG antibody, or for IgM and/or IgA structures, thereby creating monomeric antibodies comprising a single binding unit, or multimeric antibodies or multimerizing fragments or derivatives thereof having at least two bivalent binding units. In brief, nucleic acid sequences encoding the heavy and light chain variable domain sequences can be synthesized or amplified from existing molecules and inserted into vectors in the proper orientation and in frame such that upon expression, the vector will yield a full length heavy or light chain. Vectors useful for these purposes are known in the art. Such vectors can also comprise enhancer and other sequences needed to achieve expression of the desired chains. Multiple vectors or single vectors can be used. These vectors are transfected into host cells and then the chains are expressed and purified. Upon expression the chains form fully functional multimeric binding molecules, as has been reported in the literature. The fully assembled multimeric binding molecules can then be purified by standard methods. The expression and purification processes can be performed at commercial scale, if needed.

The disclosure further provides a composition comprising two or more polynucleotides, where the two or more polynucleotides collectively can encode an antigen-binding domain or an antibody, e.g., a monomeric, dimeric, hexameric, or pentameric antibody as described herein. In certain aspects the composition can include a polynucleotide encoding an IgG, IgM and/or IgA heavy chain or fragment thereof, e.g., a human IgG, IgM, or IgA heavy chain as described above where the IgG, IgM, and/or IgA heavy chain comprises at least the provided VH of a GITR antigen-binding domain as provided herein, and a polynucleotide encoding a light chain or fragment thereof, e.g., a human kappa or lambda light chain that comprises at least the provided VL of a GITR antigen-binding domain as provided herein. A polynucleotide composition as provided can further include a polynucleotide encoding a J chain, e.g., a human J chain, or a fragment, variant, or derivative thereof. In certain aspects the polynucleotides making up a composition as provided herein can be situated on two, three, or more separate vectors, e.g., expression vectors. Such vectors are provided by the disclosure. In certain aspects two or more of the polynucleotides making up a composition as provided herein can be situated on a single vector, e.g., an expression vector. Such a vector is provided by the disclosure.

The disclosure further provides a host cell, e.g., a prokaryotic or eukaryotic host cell, comprising a polynucleotide or two or more polynucleotides encoding a monomeric, dimeric, pentameric, or hexameric GITR agonist antibody or fragment or derivative thereof comprising an antigen-binding domain as provided herein, or any subunit thereof, a polynucleotide composition as provided herein, or a vector or two, three, or more vectors that collectively encode a monomeric, dimeric, pentameric, or hexameric GITR agonist antibody or fragment or derivative thereof comprising an antigen-binding domain as provided herein, or any subunit thereof. In certain aspects a host cell provided by the disclosure can express a monomeric, dimeric, pentameric, or hexameric GITR agonist antibody or fragment or derivative thereof comprising an antigen-binding domain as provided by this disclosure, or a subunit thereof.

In a related aspect, the disclosure provides a method of producing a monomeric, dimeric, pentameric, or hexameric GITR agonist antibody or fragment or derivative thereof comprising an antigen-binding domain as provided by this disclosure, where the method comprises culturing a host cell as described above and recovering the binding molecule.

### Methods of Use

This disclosure provides methods for activating signal transduction in cells that express GITR using a monomeric, dimeric, pentameric, or hexameric GITR agonist antibody or fragment or derivative thereof comprising an antigen-binding domain as provided herein. Likewise, the disclosure provides a monomeric, dimeric, pentameric, or hexameric GITR agonist antibody or fragment or derivative thereof comprising an antigen-binding domain as provided herein for use in activating signal transduction in cells that express GITR. Likewise, the disclosure provides use of a monomeric, dimeric, pentameric, or hexameric GITR agonist antibody or fragment or derivative thereof comprising an antigen-binding domain as provided herein in the preparation of a medicament for activating signal transduction in cells that express GITR. In certain aspects, upon activation of the receptors by the binding of a monomeric, dimeric, pentameric, or hexameric GITR agonist antibody or fragment or derivative thereof comprising an antigen-binding domain as provided herein to three or more receptor monomers, the cell, e.g., a T cell, e.g., a Treg or an activated effector CTL, can trigger a signal transduction pathway in the cell and thereby can induce anti-tumor immunity. In certain aspects the use of a monomeric, dimeric, pentameric, or hexameric GITR agonist antibody or fragment or derivative thereof comprising an antigen-binding domain as provided herein can provide potent T cell activation and in turn can induce potent anti-tumor immunity through, e.g., cytokine release, CTL proliferation, killing of tumor cells, and/or interruption of the suppressive effect of Treg cells in the tumor microenvironment.

In certain aspects, this disclosure provides a method for activating a cell, e.g., a T cell, e.g., a Treg or an activated effector CTL that expresses GITR, where the method includes contacting a GITR-expressing cell with a monomeric, dimeric, pentameric, or hexameric GITR agonist antibody or fragment or derivative thereof comprising an antigen-binding domain as described herein, where the antibody or fragment or derivative thereof can trigger activation of the GITR-expressing cell. Where the cell is a CTL, “activation” can include, without limitation, increased surface expression of GITR, proliferation, production of proinflammatory cytokines, resistance to the inhibitory effects of CD4+ CD25+ FoxP3+ Treg cells, and/or enhanced killing of tumor cells. Where the cell is a Treg, “activation” can include, without limitation, interference with the cell's ability to suppress anti-tumor immunity in the tumor microenvironment. In certain aspects contacting a GITR-expressing cell with a monomeric, dimeric, pentameric, or hexameric GITR agonist antibody or fragment or derivative thereof comprising an antigen-binding domain as provided herein can induce increased GITR expression, and multimerization of GITR on the cell surface. Likewise, the disclosure provides a monomeric, dimeric, pentameric, or hexameric GITR agonist antibody or fragment or derivative thereof comprising an antigen-binding domain as provided herein for use in activating a cell, e.g., a T cell, e.g., a Treg or an activated effector CTL that expresses GITR. Likewise, the disclosure provides use of a monomeric, dimeric, pentameric, or hexameric GITR agonist antibody or fragment or derivative thereof comprising an antigen-binding domain as provided herein in the preparation of a medicament for activating a cell, e.g., a T cell, e.g., a Treg or an activated effector CTL that expresses GITR.

In yet another aspect a monomeric, dimeric, pentameric, or hexameric GITR agonist antibody or fragment or derivative thereof comprising an antigen-binding domain as provided herein can facilitate cancer treatment, e.g., by slowing tumor growth, stalling tumor growth, or reducing the size of existing tumors, when administered as an effective dose to a subject in need of cancer treatment. The disclosure provides a method of treating cancer comprising administering to a subject in need of treatment an effective dose of a monomeric, dimeric, pentameric, or hexameric GITR agonist antibody or fragment or derivative thereof comprising an antigen-binding domain as provided herein. Likewise, the disclosure provides a monomeric, dimeric, pentameric, or hexameric GITR agonist antibody or fragment or derivative thereof comprising an antigen-binding domain as provided herein for use in treating cancer. Likewise, the disclosure provides use of a monomeric, dimeric, pentameric, or hexameric GITR agonist antibody or fragment or derivative thereof comprising an antigen-binding domain as provided herein in the preparation of a medicament for treating cancer.

The terms “cancer”, “tumor”, “cancerous”, and “malignant” refer to or describe the physiological condition in mammals that is typically characterized by unregulated cell growth. Examples of cancers include but are not limited to, carcinoma including adenocarcinomas, lymphomas, blastomas, melanomas, sarcomas, and leukemias. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer, colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, various types of head and neck cancer including, but not limited to, squamous cell cancers, and cancers of mucinous origins, such as, mucinous ovarian cancer, cholangiocarcinoma (liver) and renal papillary carcinoma.

This disclosure further provides a method of preventing or treating a cancer in a subject in need thereof, comprising administering to the subject an effective amount of a monomeric, dimeric, pentameric, or hexameric GITR agonist antibody or fragment or derivative thereof comprising an antigen-binding domain as provided herein, a composition or formulation comprising the binding molecule, or a polynucleotide, a vector, or a host cell as described herein.

By “therapeutically effective dose or amount” or “effective amount” is intended an amount of a monomeric, dimeric, pentameric, or hexameric GITR agonist antibody or fragment or derivative thereof comprising an antigen-binding domain as provided herein, that when administered brings about a positive immunotherapeutic response with respect to treatment of a cancer patient.

Effective doses of compositions for treatment of cancer vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages can be titrated using routine methods known to those of skill in the art to optimize safety and efficacy.

The subject to be treated can be any animal, e.g., mammal, in need of treatment, in certain aspects, the subject is a human subject.

In its simplest form, a preparation to be administered to a subject is a monomeric, dimeric, pentameric, or hexameric GITR agonist antibody or fragment or derivative thereof comprising an antigen-binding domain as provided herein administered in conventional dosage form, which can be combined with a pharmaceutical excipient, carrier or diluent as described elsewhere herein.

In certain aspects a monomeric, dimeric, pentameric, or hexameric GITR agonist antibody or fragment or derivative thereof comprising an antigen-binding domain as provided herein can be administered in combination with other cancer therapies, including, but not limited to chemotherapy, radiation therapy, or other immune modulating therapies such as cancer vaccines, immune checkpoint blockade inhibitors, immunostimulatory agents, or adoptive cell transfer such as CAR-T cells.

The compositions of the disclosure can be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In certain aspects, a GITR agonist antibody as provided herein can be introduced locally into a tumor, or in the vicinity of a tumor cell, e.g., within the tumor microenvironment (TME).

As noted above, all types of tumors are potentially amenable to treatment by this approach including, without limitation, carcinoma of the breast, lung, pancreas, ovary, kidney, colon and bladder, as well as melanomas, sarcomas and lymphomas. Mucosal distribution could be beneficial for certain cancers, e.g., lung cancer, ovarian cancer, colorectal cancer, or squamous cell carcinoma. A GITR agonist antibody as provided herein need not contact the cancer cells or tumor itself to be effective, so it is important to note that the methods of treatment provided herein can be just as effective on cancer cells that do not express GITR as it can be on cancer cells that do express GITR.

### Pharmaceutical Compositions and Administration Methods

Methods of preparing and administering a monomeric, dimeric, pentameric, or hexameric GITR agonist antibody or fragment or derivative thereof comprising an antigen-binding domain as provided herein to a subject in need thereof are well known to or are readily determined by those skilled in the art in view of this disclosure. The route of administration can be, for example, intratumoral, oral, parenteral, by inhalation or topical. The term parenteral as used herein includes, e.g., intravenous, intraarterial, intraperitoneal, intramuscular, subcutaneous, rectal, or vaginal administration. While these forms of administration are contemplated as suitable forms, another example of a form for administration would be a solution for injection, in particular for intratumoral, intravenous, or intraarterial injection or drip. A suitable pharmaceutical composition can comprise a buffer (e.g. acetate, phosphate or citrate buffer), a surfactant (e.g. polysorbate), optionally a stabilizer agent (e.g. human albumin), etc.

As discussed herein, a monomeric, dimeric, pentameric, or hexameric GITR agonist antibody or fragment or derivative thereof comprising an antigen-binding domain as provided herein can be administered in a pharmaceutically effective amount for the in vivo immuno therapeutic treatment of cancers. In this regard, it will be appreciated that the disclosed binding molecules can be formulated so as to facilitate administration and promote stability of the active agent. Pharmaceutical compositions accordingly can comprise a pharmaceutically acceptable, non-toxic, sterile carrier such as physiological saline, non-toxic buffers, preservatives and the like. A pharmaceutically effective amount of a monomeric, dimeric, pentameric, or hexameric GITR agonist antibody or fragment or derivative thereof comprising an antigen-binding domain as provided herein means an amount sufficient to achieve effective binding to a target and to achieve a therapeutic benefit. Suitable formulations are described in Remington's Pharmaceutical Sciences (Mack Publishing Co.) 16th ed. (1980).

Certain pharmaceutical compositions provided herein can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of a monomeric, dimeric, pentameric, or hexameric GITR agonist antibody or fragment or derivative thereof comprising an antigen-binding domain as provided herein that can be combined with carrier materials to produce a single dosage form will vary depending, e.g., upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, a monomeric, dimeric, pentameric, or hexameric GITR agonist antibody or fragment or derivative thereof comprising an antigen-binding domain as provided herein can be administered to a subject in need of therapy in an amount sufficient to produce a therapeutic effect. A monomeric, dimeric, pentameric, or hexameric GITR agonist antibody or fragment or derivative thereof comprising an antigen-binding domain as provided herein can be administered to the subject in a conventional dosage form prepared by combining the antibody of the disclosure with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

This disclosure also provides for the use of a monomeric, dimeric, pentameric, or hexameric GITR agonist antibody or fragment or derivative thereof comprising an antigen-binding domain as provided herein in the manufacture of a medicament for treating, preventing, or managing cancer.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S. C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

All of the references cited above, as well as all references cited herein, are incorporated herein by reference in their entireties.

The following examples are offered by way of illustration and not by way of limitation.

### EXAMPLES

### Example 1

### Identification of Anti-GITR Antibodies from Phage Display Library

The SuperHuman-2.0 scFv Antibody Library (available from Distributed Bio, South San Francisco, Calif.) was used to perform selection with human, cynomolgus or mouse GITR proteins. Selected phage clones from overnight growth were inoculated into wells in 96 well deep well plates containing 1 mL of medium. scFv expression was induced by addition of IPTG (Teknova) to a final concentration of 1 mM. Plates were grown overnight after induction. To prepare periplasmic extracts (PPE), the plates containing the phage clones were centrifuged at 2500 rpm for 10 min, the supernatant discarded and then pellets treated with 75 μL of cold periplasmic extract buffer with appropriate concentration of protease inhibitors (Complete Mini, EDTA-free -Roche Applied Biosciences—Cat No 1836170). After shaking at room temperature for 10 min, 225 μL of cold ddH2O with added protease inhibitor (1 tablet/50 mL) was added per well and mixed by pipetting up and down. PPE extraction was completed by incubation at room temperature for an additional 1 hr. The plates were then centrifuged at 4000 rpm for 20 minutes and 200 μL of PPE supernatant was collected from each well and transferred to a new plate which was then blocked with equal volume of 4% milk (2% final) to prepare blocked PPE extracts. In the meantime, Maxisorp plates (Nunc 442404) coated with relevant antigen (human, mouse or cynomolgus monkey His6-GITR) at 50 μl/well of 1 μg/mL antigen or 2% BSA in PBS as control were prepared for ELISA analyses. Next, 50 μL of each blocked PPE extract was transferred to the pre-blocked ELISA plates coated with the relevant antigens. After incubation and washing 3× with PBS, 50 μL of anti-V5 tag antibody (tag included in the phage display constructs, Invitrogen Cat No R960-25) at 1:5000 dilution in 2% milk was added and the plates were incubated for 1 hr. After washing with three 1× PBST washes, HRP goat anti-mouse antibody (Jackson Immuno Research Cat No 115-035-003/071) at 1:5000 dilution in 2% milk was added for detection of bound anti-V5 antibody and incubated for 45 minute. After further washing 3× with PBST, 50 μl/well of TMB substrate was added and the plates were developed until positive controls were visible. The reaction was stopped with 50 μl/well of 2N H2SO4. Plates were read at 450 nm to determine ELISA signal from bound PPE derived scFv. ScFvs with at least 5-fold better binding to the hGITR-His over a background His fusion protein were identified, resulting in the original identification of 128 unique antibodies. A second similar screen was also carried out.

PPE were also screened for antibodies binding to human and cynomolgus monkey GITR using the CARTERRA® LSA monoclonal antibody screening platform according to manufacturer's specifications to measure values for dissociation constants (Kd). Forty-eight (48) of the unique scFvs, GITR-Mab-1 to GITR-Mab-48, were chosen for further processing. The binding characteristics of the 24 scFvs, to human, cynomolgus monkey, and mouse GITR-His, expressed as x-fold over background are shown in Table 2. The disassociation constants of the scFvs for human GITR (in nM), determined by CARTERRA® LSA, are also shown in Table 2. All 24 of the selected anti-GITR scFvs bound well to human GITR-His and cyno GITR-His. Only one of the selected antibodies, GITR-Mab-4, showed appreciable binding to mouse GITR-His.

TABLE 2
Binding Characteristics of Anti-GITR ScFvs

| | hGITR- | cGITR- | mGITR- | hGITR- |
|---|---|---|---|---|
| NAME | His/BKG | His/BKG | His/BKG | His KD (nM) |
| GITR-Mab-1 | 13.2 | 15.8 | 1.1 | ND |
| GITR-Mab-2 | 20.1 | 25.2 | 1.1 | 16.3 |
| GITR-Mab-3 | 12.1 | 11.1 | 1.0 | 10.5 |
| GITR-Mab-4 | 9.6 | 10.3 | 16.1 | ND |
| GITR-Mab-5 | 16.3 | 8.0 | 1.2 | 8.3 |
| GITR-Mab-6 | 35.8 | 7.5 | 1.0 | 0.1 |
| GITR-Mab-7 | 16.4 | 9.0 | 1.0 | 11.5 |
| GITR-Mab-8 | 9.6 | 16.1 | 1.4 | 49.6 |
| GITR-Mab-9 | 12.8 | 11.6 | 1.1 | 3.3 |
| GITR-Mab-10 | 34.2 | 29.4 | 1.1 | 5.3 |
| GITR-Mab-11 | 21.8 | 15.6 | 1.1 | 1.4 |
| GITR-Mab-12 | 37.8 | 16.9 | 1.0 | 7.5 |
| GITR-Mab-13 | 25.4 | 9.4 | 1.1 | 2.6 |
| GITR-Mab-14 | 34.0 | 11.5 | 0.9 | 30.3 |
| GITR-Mab-15 | 11.4 | 12.3 | 1.2 | 52.1 |
| GITR-Mab-16 | 24.1 | 21.6 | 0.8 | 0.8 |
| GITR-Mab-17 | 18.9 | 10.9 | 1.1 | 24.5 |
| GITR-Mab-18 | 17.9 | 17.7 | 1.0 | 1.0 |
| GITR-Mab-19 | 10.0 | 24.6 | 1.3 | 10.8 |
| GITR-Mab-20 | 24.7 | 15.3 | 1.1 | 7.8 |
| GITR-Mab-21 | 13.5 | 21.2 | 1.2 | 2.9 |
| GITR-Mab-22 | 25.7 | 23.3 | 1.2 | 2.3 |
| GITR-Mab-23 | 35.6 | 23.2 | 0.9 | ND |
| GITR-Mab-24 | 19.0 | 5.7 | 1.7 | 38.9 |
| GITR-Mab-25 | 22.2 | 1.1 | 1.1 | ND |
| GITR-Mab-26 | 12.7 | 0.8 | 1.1 | ND |
| GITR-Mab-27 | 24.6 | 1.0 | 1.3 | ND |
| GITR-Mab-28 | 11.5 | 1.0 | 1.0 | ND |
| GITR-Mab-29 | 12.4 | 1.1 | 1.0 | ND |
| GITR-Mab-30 | 18.7 | 1.0 | 1.0 | ND |
| GITR-Mab-31 | 17.7 | 1.2 | 1.2 | ND |
| GITR-Mab-32 | 18.0 | 4.9 | 1.0 | ND |
| GITR-Mab-33 | 17.7 | 1.3 | 1.0 | ND |
| GITR-Mab-34 | 7.7 | 1.1 | 1.2 | ND |
| GITR-Mab-35 | 25.3 | 0.9 | 0.9 | ND |
| GITR-Mab-36 | 8.8 | 1.4 | 0.9 | ND |
| GITR-Mab-37 | 13.2 | 11.7 | 13.7 | ND |
| GITR-Mab-38 | 2.9 | 3.0 | 3.8 | ND |
| GITR-Mab-39 | 4.2 | 4.0 | 4.4 | ND |
| GITR-Mab-40 | 13.9 | 15.5 | 23.2 | ND |
| GITR-Mab-41 | 9.9 | 10.3 | 10.4 | ND |
| GITR-Mab-42 | 5.5 | 4.2 | 1.0 | ND |
| GITR-Mab-43 | 11.7 | 12.0 | 14.6 | ND |
| GITR-Mab-44 | 12.4 | 12.3 | 15.6 | ND |
| GITR-Mab-45 | 1.4 | 3.2 | 4.5 | ND |
| GITR-Mab-46 | 4.0 | 5.3 | 6.5 | ND |
| GITR-Mab-47 | 13.0 | 21.0 | 24.9 | ND |
| GITR-Mab-48 | 9.4 | 8.9 | 9.5 | ND |

The selected scFvs were sequenced. The VH and VL amino acid sequences are shown in Table 3, and the CDR amino acid sequences are shown in Table 4.

TABLE 3
GITR Mab VH and VL Sequences

| | VH | | VL | |
|---|---|---|---|---|
| | SEQ | VH | SEQ | VL |
| Mab | ID | SEQUENCE | ID | SEQUENCE |
| GITR-Mab-1 | 1 | QVQLVQSGAE | 5 | EIVMTQSPAT |
| | | VKKPGASVKV | | LSVSPGERAT |
| | | SCKASGYTFT | | LSCRASQSVY |
| | | NYHLHWVRQA | | SNYLAWYQQK |
| | | PGQGLEWMGM | | PGQAPRLLIY |
| | | INPNDGSTTY | | GASTRATGIP |
| | | AQKFQGRVTM | | ARFSGSGSGT |
| | | TRDTSTSTVY | | EFTLTISSLQ |
| | | MELSSLRSED | | SEDFAVYYCQ |
| | | TAVYYCARST | | QYHSYPLTFG |
| | | YYYDSSGYYY | | GGTKVEIK |
| | | YYYGMDVWGQ | | |
| | | GTTVTVSS | | |
| GITR-Mab-2 | 9 | QVQLVQSGAE | 13 | DIVMTQSPLS |
| | | VKKPGSSVKV | | LPVTPGEPAS |
| | | SCKASGYRFT | | ISCRSSQSLL |
| | | GYHLHWVRQA | | HSNGYNYLDW |
| | | PGQGLEWMGG | | YLQKPGQSPQ |
| | | IIPIFGTANY | | LLIYLGSYRA |
| | | AQKFQGRVTI | | SGVPDRFSGS |
| | | TADKSTSTAY | | GSGTDFTLKI |
| | | MELSSLRSED | | SRVEAEDVGV |
| | | TAVYYCAYGV | | YYCRQALRTP |
| | | PPDPWGQGTL | | LTFGGGTKVE |
| | | VTVSS | | IK |
| GITR-Mab-3 | 17 | QVQLVQSGAE | 21 | EIVMTQSPAT |
| | | VKKPGASVKV | | LSVSPGERAT |
| | | SCKASGYTFT | | LSCRASQSVS |
| | | SYDINWVRQA | | SSYLAWYQQK |
| | | PGQGLEWMGW | | PGQAPRLLIY |
| | | ISGYNGNTNY | | GASTRATGIP |
| | | AQKFQGRVTM | | ARFSGSGSGT |
| | | TRDTSTSTVY | | EFTLTISSLQ |
| | | MELSSLRSED | | SEDFAVYYCQ |
| | | TAVYYCARSH | | QYYTTPFTFG |
| | | EYYYYYGMDV | | PGTKVDIK |
| | | WGQGTLVTVS | | |
| | | S | | |
| GITR-Mab-4 | 25 | EVQLLESGGG | 29 | DIVMTQSPLS |
| | | LVQPGGSLRL | | LPVTPGEPAS |
| | | SCAASGFTFS | | ISCRSSQSLL |
| | | SYDMHWVRQA | | HSNGYNYLDW |
| | | PGKGLEWVSA | | YLQKPGQSPQ |
| | | ISSSGGSIYY | | LLIYLGSNRA |
| | | ADSVKGRFTI | | SGVPDRFSGS |
| | | SRDNSKNTLY | | GSGTDFTLKI |
| | | LQMNSLRAED | | SRVEAEDVGV |
| | | TAVYYCARDS | | YYCMQATHSP |
| | | VWPKGPNRKY | | YTFGQGTKVE |
| | | YYYGMDVWGQ | | IK |
| | | GTKVTVSS | | |
| GITR-Mab-5 | 33 | EVQLLESGGG | 37 | DIQMTQSPSS |
| | | LVQPGGSLRL | | LSASVGDRVT |
| | | SCAASGFTFD | | ITCRASQGIK |
| | | AYAMHWVRQA | | NDLGWYQQKP |
| | | PGKGLEWVSA | | GKAPKLLIYA |
| | | IGTGGDTYYA | | ASSLQSGVPS |
| | | DSVKGRFTIS | | RFSGSGSGTD |
| | | RDNSKNTLYL | | FTLTISSLQP |
| | | QMNSLRAEDT | | EDFATYYCQQ |
| | | AVYYCARDLY | | SYSVPFTFGQ |
| | | GSGSPQYYYY | | GTKVEDC |
| | | YGMDVWGQGT | | |
| | | TVTVSS | | |
| GITR-Mab-6 | 41 | QVQLVQSGAE | 45 | DIVMTQSPLS |
| | | VKKPGASVKV | | LPVTPGEPAS |
| | | SCKASGYAFT | | ISCRSSQSLL |
| | | AYYLHWVRQA | | HSNGYNYLDW |
| | | PGQGLEWMGW | | YLQKPGQSPQ |
| | | MNPNSGNTGY | | LLIYLGSRRA |
| | | AQKFQGRVTM | | SGVPDRFSGS |
| | | TRDTSTSTVY | | GSGTDFTLKI |
| | | MELSSLRSED | | SRVEAEDVGV |
| | | TAVYYCAREG | | YYCMQGSHWP |
| | | WGYYDGGFDP | | PTFGPGTKVD |
| | | WGQGTLVTVS | | DC |
| | | S | | |
| GITR-Mab-7 | 49 | QVQLVQSGAE | 53 | DIQMTQSPSS |
| | | VKKPGASVKV | | LSASVGDRVT |
| | | SCKASGGTSS | | ITCQASQDIS |
| | | TYAVTWVRQA | | NYLNWYQQKP |
| | | PGQGLEWMGV | | GKAPKLLIYA |
| | | INPNDGSTTY | | ASSLQSGVPS |
| | | AQNFQGRVTM | | RFSGSGS |
| | | TRDTSTSTVY | | |
| | | MELSSLRSED | | |
| | | TAV | | |
| | | YYCARWTPLF | | GTDFTLTISS |
| | | GWIPDYYYYG | | LQPEDFATYY |
| | | MDVWGQGTLV | | CQQSYSTPPT |
| | | TVSS | | FGQGTRLEIK |
| GITR-Mab-8 | 57 | QVQLVQSGAE | 61 | DIVMTQSPDS |
| | | VKKPGASVKV | | LAVSLGERAT |
| | | SCKASGGTFS | | INCKSSQSVL |
| | | SYVISWVRQA | | YSSNNKNYLA |
| | | PGQGLEWMGW | | WYQQKPGQPP |
| | | MNPGSGNTGY | | KLLIYWASTR |
| | | AQKFQGRVTM | | ESGVPDRFSG |
| | | TRDTSTSTVY | | SGSGTDFTLT |
| | | MELSSLRSED | | ISSLQAEDVA |
| | | TAVYYCITDS | | VYYCQQYYNT |
| | | EDDYWGQGTL | | PYTFGQGTKV |
| | | VTVSS | | EIK |
| GITR-Mab-9 | 65 | QVQLVQSGAE | 69 | DIQMTQSPSS |
| | | VKKPGASVKV | | LSASVGDRVT |
| | | SCKASGHTFT | | ITCQASQDIG |
| | | SQYMHWVRQA | | NYLNWYQQKP |
| | | PGQGLEWVGV | | GKAPKLLIYG |
| | | INPNDGSTSY | | ASNLETGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCARDG | | GYSFPLTFGQ |
| | | AYYYDSSGYY | | GTKVEIK |
| | | RSSNFDYWGQ | | |
| | | GTLVTVSS | | |
| GITR-Mab-10 | 73 | QVQLVQSGAE | 77 | DIQMTQSPSS |
| | | VKKPGASVKV | | LSASVGDRVT |
| | | SCKASGYTFT | | ITCRASQGIS |
| | | SNYMHWVRQA | | SWLAWYQQKP |
| | | PGQGLEWMGW | | GKAPKLLIYA |
| | | MNPNSGNTAY | | ASTLQSGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCARGR | | YNSYSTFGQG |
| | | GGQLLFDYWG | | TKVEIK |
| | | QGTLVTVSS | | |
| GITR-Mab-11 | 81 | QVQLVQSGAE | 85 | DIQMTQSPSS |
| | | VKKPGASVKV | | LSASVGDRVT |
| | | SCKASGGTFS | | ITCRASQSIS |
| | | NYGISWVRQA | | SYLNWYQQKP |
| | | PGQGLEWMGI | | GKAPKLLIYA |
| | | INPTDGSTTY | | ASSLESGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCARWW | | SYSTPPTFGQ |
| | | GSGWSWYFDL | | STRLEIK |
| | | WGRGTLVTVS | | |
| | | S | | |
| GITR-Mab-12 | 89 | QVQLVQSGAE | 93 | DIQMTQSPSS |
| | | VKKPGASVKV | | LSASVGDRVT |
| | | SCKASGYTFT | | ITCRASQGIS |
| | | SYAINWVRQA | | NSLAWYQQKP |
| | | PGQGLEWMGI | | GKAPKLLIYA |
| | | LSPSGGGTSY | | ASSLQSGVPS |
| | | APKFQGRVTM | | RFSGSGSGTD |
| | | TRDTSTSTVY | | FTLTISSLQP |
| | | MELSSLRSED | | EDFATYYCQQ |
| | | TAVYYCARGP | | SYSTPFTFGP |
| | | WYFDLWGRGT | | GTKVDIK |
| | | LVTVSS | | |
| GITR-Mab-13 | 97 | QVQLVQSGAE | 101 | DIVMTQSPDS |
| | | VKKPGSSVKV | | LAVSLGERAT |
| | | SCKASGGTFS | | INCKSSQSLL |
| | | SYAISWVRQA | | YSSNNRNYLA |
| | | PGQGLEWMGG | | WYQQKPGQPP |
| | | IVPMLGSPHY | | KLLIYWASTR |
| | | AQKFQGRVTI | | ESGVPDRFSG |
| | | TADESTSTAY | | SGSGTDFTLT |
| | | MELSSLRSED | | ISSLQAEDVA |
| | | TAVYYCARGS | | VYYCQQYYST |
| | | WLVADFQHWG | | PITFGQGTRL |
| | | QGTLVTVSS | | EIK |
| GITR-Mab-14 | 105 | EVQLLESGGG | 109 | DIQMTQSPSS |
| | | LVKPGGSLRL | | LSASVGDRVT |
| | | SCAASGFRFS | | ITCQASQDIS |
| | | VYWMSWVRQA | | NYLNWYQQKP |
| | | PGKGLEWVSG | | GKAPKLLIFD |
| | | ISGSGGTTYY | | ASSLEAGVPS |
| | | ADSVKGRFTI | | RFSGSGSGTD |
| | | SRDDSKNTLY | | FTLTISSLQP |
| | | LQMNSLKTED | | EDFATYYCQQ |
| | | TAVYYCARVR | | ANSFPPTFGQ |
| | | RDGYNYNFDY | | GTEVEIK |
| | | WGQGTLVTVS | | |
| | | S | | |
| GITR-Mab-15 | 113 | QVQLVQSGAE | 117 | DIVMTQSPDS |
| | | VKKPGSSVKV | | LAVSLGERAT |
| | | SCKASGFTFT | | INCKSSQSVL |
| | | TSAVQWVRQA | | YNSNNLNYLA |
| | | PGQGLEWMGG | | WYQQKPGQPP |
| | | IIPIFGTANY | | KLLIYWASIR |
| | | AQKFQGRVTI | | ESGVPDRFSG |
| | | TADESTSTAY | | SGSGTDFTLT |
| | | MELSSLRSED | | ISSLQAEDVA |
| | | TAVYYCAKGS | | VYYCQQYYST |
| | | GYEFPGGSEY | | PLTFGQGTKV |
| | | FQHWGQGTLV | | EIK |
| | | TVSS | | |
| GITR-Mab-16 | 121 | QVQLVQSGAE | 125 | DIQMTQSPSS |
| | | VKKPGASVKV | | LSASVGDRVT |
| | | SCKASGGTLS | | ITCRASRSIS |
| | | SYAISWVRQA | | NYLNWYQQKP |
| | | PGQGLEWMGW | | GKAPKLLIYG |
| | | IDPNSGGTNY | | ASSLQSGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCARQG | | SYSTLVTFGQ |
| | | LLWFGESGSI | | GTKVEIK |
| | | YYYYGMDVWG | | |
| | | QGTTVTVSS | | |
| GITR-Mab-17 | 129 | QVQLVQSGAE | 133 | DIVMTQSPDS |
| | | VKKPGASVKV | | LAVSLGERAT |
| | | SCKASGYTFT | | INCKSSQSVL |
| | | SYGISWVRQA | | YSSNNKNYLA |
| | | PGQGLE | | WYQ |
| | | WMGWMNPNSG | | QKPGQPPKLL |
| | | NTGYAQKFQG | | IYWASTREPG |
| | | RVTMTRDTST | | VPDRFSGSGS |
| | | STVYMELSSL | | GTDFTLTISS |
| | | RSEDTAVYYC | | LQAEDVAVYY |
| | | ARLGLWFGEY | | CQQYYSTPLT |
| | | QYYFDYWGQG | | FGQGTKVEIK |
| | | TLVTVSS | | |
| GITR-Mab-18 | 137 | EVQLLESGGG | 141 | DIQMTQSPSS |
| | | LVKPGGSLRL | | LSASVGDRVT |
| | | SCAASGFTFS | | ITCRASESIS |
| | | SYWMSWVRQA | | TWLAWYQQKP |
| | | PGKGLEWVSG | | GKAPKLLIYA |
| | | ISWNGGTVGY | | ASSLQSGVPS |
| | | ADSVKGRFTI | | RFSGSGSGTD |
| | | SRDDSKNTLY | | FTLTISSLQP |
| | | LQMNSLKTED | | EDFATYYCQQ |
| | | TAVYYCAKLG | | SYTLPLTFGG |
| | | IAVKSHWYFD | | GTKLEIK |
| | | LWGRGTLVTV | | |
| | | SS | | |
| GITR-Mab-19 | 145 | QVQLVQSGAE | 149 | DIQMTQSPSS |
| | | VKKPGASVKV | | LSASVGDRVT |
| | | SCKASGYTFT | | ITCRASQDIV |
| | | SYDINWVRQA | | NWLAWYQQKP |
| | | PGQGLEWVGI | | GKAPKLLIYA |
| | | INPSGGSTSY | | ASSLQSGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCARAS | | YYSYPLTFGQ |
| | | SGGYYYYYGM | | GTRLEIK |
| | | DVWGQGTTVT | | |
| | | VSS | | |
| GITR-Mab-20 | 153 | QVQLVQSGAE | 157 | DIQMTQSPSS |
| | | VKKPGASVKV | | LSASVGDRVT |
| | | SCKASGGTFS | | ITCRASQSIS |
| | | NYAISWVRQA | | TYLNWYQQKP |
| | | PGQGLEWMGV | | GKAPKLLIYD |
| | | INPRGGSTTY | | ASNLETGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCARDY | | SYSTPLTFGG |
| | | SEPYYGMDVW | | GTKVEIK |
| | | GQGTTVTVSS | | |
| GITR-Mab-21 | 161 | EVQLLESGGG | 165 | DIQMTQSPSS |
| | | LVQPGGSLRL | | LSASVGDRVT |
| | | SCAASGFTFS | | ITCQASQDIS |
| | | NHYMSWVRQA | | NYLNWYQQKP |
| | | PGKGLEWVAV | | GKAPKLLIYA |
| | | IALDGSYRYY | | ASSLQSGVPS |
| | | ADSVKGRFTI | | RFSGSGSGTD |
| | | SRDNSKNTLY | | FTLTISSLQP |
| | | LQMNSLRAED | | EDFATYYCQQ |
| | | TAVYYCARVG | | SYNSPRVYTF |
| | | PGGMDVRGQG | | GQGTKVEIK |
| | | TTVTVSS | | |
| GITR-Mab-22 | 169 | QVQLVQSGAE | 173 | DIQMTQSPSS |
| | | VKKPGASVKV | | LSASVGDRVT |
| | | SCKASGGRFS | | ITCRASQSIS |
| | | TYALSWVRQA | | NWLAWYQQKP |
| | | PGQGLEWMGI | | GKAPKLLIYA |
| | | INPTDGSTTY | | ASTLQSGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCARDV | | SYSVPFTFGP |
| | | YSSSWYSDYW | | GTKVDIK |
| | | GQGTLVTVSS | | |
| GITR-Mab-23 | 177 | EVQLLESGGG | 181 | DIQMTQSPSS |
| | | LVQPGGSLRL | | LSASVGDRVT |
| | | SCAASGFPFS | | ITCRASQAIS |
| | | TYAIHWVRQA | | NSLAWYQQKP |
| | | PGKGLEWVSA | | GKAPKLLIYA |
| | | ISGSGGSTYY | | ASTLQRGVPS |
| | | ADSVKGRFTI | | RFSGSGSGTD |
| | | SRDNSKNTLY | | FTLTISSLQP |
| | | LQMNSLRAED | | EDFATYYCQQ |
| | | TAVYYCAGPD | | YYSTPYTFGQ |
| | | WYFDLWGRGT | | GTKLEIK |
| | | LVTVSS | | |
| GITR-Mab-24 | 185 | QVQLVQSGAE | 189 | DIQMTQSPSS |
| | | VKKPGASVKV | | LSASVGDRVT |
| | | SCKASGGTFN | | ITCRASQNIT |
| | | NYAINWVRQA | | NWLAWYQQKP |
| | | PGQGLEWMGT | | GKAPKLLIYA |
| | | INPRDGDTTY | | ASSLQSGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCARVG | | AYSFPWTFGQ |
| | | YYGSGSYYSY | | GTKVEIK |
| | | YGMDVWGQGT | | |
| | | TVTVSS | | |
| GITR-Mab-25 | 203 | QVQLVQSGAE | 207 | DIQMTQSPSS |
| | | VKKPGASVKV | | LSASVGDRVT |
| | | SCKASGGTFS | | ITCRTSQSIR |
| | | SYAISWVRQA | | RYLNWYQQKP |
| | | PGQGLEWMGW | | GKAPKLLIYA |
| | | VSGYNGNANY | | ASSLQSGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCARDR | | SYSIPWTFGP |
| | | VESGYSYHDA | | GTKVDIK |
| | | FDIWGQGTMV | | |
| | | TVSS | | |
| GITR-Mab-26 | 211 | QVQLVQSGAE | 215 | DIQMTQSPSS |
| | | VKKPGASVKV | | LSASVGDRVT |
| | | SCKASGYTFT | | ITCQASQSIS |
| | | SYGISWVRQA | | RWLAWYQQKP |
| | | PGQGLEWMGW | | GKAPKLLIYS |
| | | MNPNNGNTVY | | ASSLQSGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCAKDS | | SYSTPWTFGQ |
| | | DWYGAFDIWG | | GTKLEIK |
| | | QGTMVTVSS | | |
| GITR-Mab-27 | 219 | QVQLVQSGAE | 223 | DIQMTQSPSS |
| | | VKKPGASVKV | | LSASVGDRVT |
| | | SCKASGYTFT | | ITCQASHDID |
| | | SYDINWVRQA | | NYLNWYQQKP |
| | | PGQGLEWIGW | | GKAPKLLIYA |
| | | MNTNSGDTGY | | ASNLQSGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCARQA | | ANSFPLTFGP |
| | | YSSSWYWYGW | | GTKVDIK |
| | | FDPWGQGTLV | | |
| | | TVSS | | |
| GITR-Mab-28 | 227 | QVQLVQSGAE | 231 | EIVMTQSPAT |
| | | VKKPGASVKV | | LSVSPGERAT |
| | | SCKASGYTFP | | LSCRASQSVS |
| | | NYGITWVRQA | | SNYLAWYQQK |
| | | PGQGLEWMGW | | PGQAPRLLIY |
| | | MNPNSGYTGY | | GASTRATGIP |
| | | AQNFQGRVTM | | ARFSGSGSGT |
| | | TRDTSTSTVY | | EFTLTISSLQ |
| | | MELSSLRSED | | SEDFAVYYCQ |
| | | TAVYYCARGM | | QYHTYPPTFG |
| | | PGRGFDPWGQ | | QGTKLEIK |
| | | GTLVTVSS | | |
| GITR-Mab-29 | 235 | QVQLVQSGAE | 239 | DIQMTQSPSS |
| | | VKKPGASVKV | | LSASVGDRVT |
| | | SCKASGYTFS | | ITCRASQSIS |
| | | SDYIHWVRQA | | SYLNWYQQKP |
| | | PGQGLEWMGR | | GKAPKLLIYG |
| | | INPSGGSTLY | | ASSLQSGVPS |
| | | ARRFQGRVTM | | RFSGSGSGTD |
| | | TRDTSTSTVY | | FTLTISSLQP |
| | | MELSSLRSED | | EDFATYYCQQ |
| | | TAVYYCARER | | SYRTPLTFGG |
| | | GAADTWYFDL | | GTKVEIK |
| | | WGRGTLVTVS | | |
| | | S | | |
| GITR-Mab-30 | 243 | QVQLVQSGAE | 247 | DIQMTQSPSS |
| | | VKKPGASVKV | | LSASVGDRVT |
| | | SCKASGGTFS | | ITCRASQSVS |
| | | SYAISWVRQA | | TYLNWYQQKP |
| | | PGQGLEWMGI | | GKAPKLLIYA |
| | | IDPTGGATAY | | ASSLQSGVPS |
| | | AQEFQGRVTM | | RFSGSGSGTD |
| | | TRDTSTSTVY | | FTLTISSLQP |
| | | MELSSLRSED | | EDFATYYCQQ |
| | | TAVYYCARVG | | SYSTPLTFGG |
| | | YYYGMDVWGQ | | GTKVEIK |
| | | GTMVTVSS | | |
| GITR-Mab-31 | 251 | QVQLVQSGAE | 255 | EIVMTQSPAT |
| | | VKKPGSSVKV | | LSVSPGERAT |
| | | SCKASGGTFS | | LSCRASQTVG |
| | | SYAISWVRQA | | SRYLAWYQQK |
| | | PGQGLEWMGG | | PGQAPRLLIY |
| | | IIPISSATSI | | GASTRATGIP |
| | | PQKFQGRVTI | | ARFSGSGSGT |
| | | TADESTSTAY | | EFTLTISSLQ |
| | | MELSSLRSED | | SEDFAVYYCQ |
| | | TAVYYCARSY | | QYYSTPWTFG |
| | | DSRYYGMDVW | | QGTRLEIK |
| | | GQGTTVTVSS | | |
| GITR-Mab-32 | 259 | QVQLVQSGAE | 263 | DIQMTQSPSS |
| | | VKKPGASVKV | | LSASVGDRVT |
| | | SCKASGGTFS | | ITCRVSQGIS |
| | | SYAISWVRQA | | NSLAWYQQKP |
| | | PGQGLEWMGW | | GKAPKLLIYA |
| | | MNPNSGDTGY | | ASSLQSGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCARGP | | SYSTVYTFGQ |
| | | WYFDLWGRGT | | GTKLEIK |
| | | LVTVSS | | |
| GITR-Mab-33 | 267 | EVQLLESGGG | 271 | DIQMTQSPSS |
| | | LVKPGGSLRL | | LSASVGDRVT |
| | | SCAASGFMFS | | ITCRASQSIS |
| | | SYSMNWVRQA | | TWLAWYQQKP |
| | | PGKGLEWVSY | | GKAPKLLIYD |
| | | ISGNSGYTNY | | ASNLETGVPS |
| | | ADSVKGRFTI | | RFSGSGSGTD |
| | | SRDDSKNTLY | | FTLTISSLQP |
| | | LQMNSLKTED | | EDFATYYCQQ |
| | | TAVYYCARRL | | AYRFPVAFGG |
| | | HGMDVWGQGT | | GTKVEIK |
| | | TVTVSS | | |
| GITR-Mab-34 | 275 | EVQLLESGGG | 279 | DIQMTQSPSS |
| | | LVQPGGSLRL | | LSASVGDRVT |
| | | SCAASGFTFS | | ITCRASQDIR |
| | | SYWMHWVRQA | | NYLAWYQQKP |
| | | PGKGLEWVSV | | GKAPKLLIYT |
| | | ISNSGGATYY | | ASTLQSGVPS |
| | | ADSVKGRFTI | | RFSGSGSGTD |
| | | SRDNSKNTLY | | FTLTISSLQP |
| | | LQMNSLRAED | | EDFATYYCQQ |
| | | TAVYYCAREG | | TFRTPLTFGG |
| | | WGYGMDVWGQ | | GTKVEIK |
| | | GTTVTVSS | | |
| GITR-Mab-35 | 283 | QVQLVQSGAE | 287 | DIQMTQSPSS |
| | | VKKPGASVKV | | LSASVGDRVT |
| | | SCKASGYTFT | | ITCQASQGIS |
| | | NYYMHWVRQA | | SYLNWYQQKP |
| | | PGQGLEWMGL | | GKAPKLLIYA |
| | | ITPSGGRTKY | | ASTLQSGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCAREM | | SYSTPLTFGG |
| | | EYSSSWYWFD | | GTKVEIK |
| | | PWGQGTLVTV | | |
| | | SS | | |
| GITR-Mab-36 | 291 | QVQLVQSGAE | 295 | DIQMTQSPSS |
| | | VKKPGASVKV | | LSASVGDRVT |
| | | SCKASGYTFT | | ITCRASQSIS |
| | | RNYIHWVRQA | | SYLNWYQQKP |
| | | PGQGLEWMGW | | GKAPKLLIYA |
| | | INPKSGGTKY | | ASSLQSGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCARES | | SYSTPLTFGG |
| | | GWATEYWYFD | | GTKVEIK |
| | | LWGRGTLVTV | | |
| | | SS | | |
| GITR-Mab-37 | 299 | QVQLVQSGAE | 303 | DIVMTQSPDS |
| | | VKKPGASVKV | | LAVSLGERAT |
| | | SCKASGYTFT | | INCKSSQSIF |
| | | SYYMHWVRQA | | YSSNSKNYLA |
| | | PGQGLEWMGW | | WYQQKPGQPP |
| | | INPNSGGTNY | | KLLIYWASTR |
| | | AQKFQGRVTM | | ESGVPDRFSG |
| | | TRDTSTSTVY | | SGSGTDFTLT |
| | | MELSSLRSED | | ISSLQAEDVA |
| | | TAVYYCASPG | | VYYCQQYYST |
| | | DYCSGGSCYS | | PLTFGPGTKV |
| | | DDAFDIWGQG | | DIK |
| | | TMVTVSS | | |
| GITR-Mab-38 | 307 | QVQLVQSGAE | 311 | DIQMTQSPSS |
| | | VKKPGASVKV | | LSASVGDRVT |
| | | SCKASGGTFR | | ITCRASQGIS |
| | | NYAINWVRQA | | NYLAWYQQKP |
| | | PGQGLEWMGW | | GKAPKLLIYS |
| | | MNPNSGNTGY | | ASNLQSGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCARED | | GYSSRYTFGQ |
| | | VDTASQAYFD | | GTKLEIK |
| | | YWGQGTLVTV | | |
| | | SS | | |
| GITR-Mab-39 | 315 | EVQLLESGGG | 319 | DIQMTQSPSS |
| | | LVKPGGSLRL | | LSASVGDRVT |
| | | SCAASGFTFS | | ITCRASQSIS |
| | | SYWMSWVRQA | | DWLAWYQQKP |
| | | PGKGLEWVSS | | GKAPKLLIYE |
| | | ISWSSTYIYY | | ASKLATGVPS |
| | | ADSVKGRFTI | | RFSGSGSGTD |
| | | SRDDSKNTLY | | FTLTISSLQP |
| | | LQMNSLKTED | | EDFATYYCQQ |
| | | TAVYYCARDG | | SYTTPLTFGG |
| | | QLGHWHFDLW | | GTKVEIK |
| | | GRGTLVTVSS | | |
| GITR-Mab-40 | 323 | EVQLLESGGG | 327 | DIVMTQSPLS |
| | | LVQPGGSLRL | | LPVTPGEPAS |
| | | SCAASGFTFR | | ISCRSSQSLL |
| | | NHWMSWVRQA | | HSNGYNYLDW |
| | | PGKGLEWVSG | | YLQKPGQSPQ |
| | | ISWNSGSBDY | | LLIYLGSNRA |
| | | ADSVKGRFTI | | SGVPDRFSGS |
| | | SRDNSKNTLY | | GSGTDFTLKI |
| | | LQMNSLRAED | | SRVEAEDVGV |
| | | TAVYYCAREE | | YYCMQGTHWP |
| | | YATFDYWGQG | | PTFGPGTKVD |
| | | TLVTVSS | | IK |
| GITR-Mab-41 | 331 | QVQLVQSGAE | 335 | DIQMTQSPSS |
| | | VKKPGASVKV | | LSASVGDRVT |
| | | SCKASGGSFS | | ITCRASQDIR |
| | | DYAVSWVRQA | | NDLAWYQQKP |
| | | PGQGLEWMGW | | GKAPKLLIYA |
| | | MNPNSGNTGY | | ASSLQSGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCAREL | | SFSTFYTFGQ |
| | | VRDGYNFALD | | GTKLEIK |
| | | YWGQGTLVTV | | |
| | | SS | | |
| GITR-Mab-42 | 339 | QVQLVQSGAE | 343 | DIVMTQSPLS |
| | | VKKPGASVKV | | LPVTPGEPAS |
| | | SCKASGYIFT | | ISCRSSQSLL |
| | | NYWIQWVRQA | | HSNGYNYLDW |
| | | PGQGLEWMGW | | YLQKPGQSPQ |
| | | INPHSGATKY | | LLIYLGSNRA |
| | | AERFQGRVTM | | SGVPDRFSGS |
| | | TRDTSTSTVY | | GSGTDFTLKI |
| | | MELSSLRSED | | SRVEAEDVGV |
| | | TAVYYCARGL | | YYCMQALQTP |
| | | GSAFDIWGQG | | LTFGGGTKVE |
| | | TMVTVSS | | IK |
| GITR-Mab-43 | 347 | QVQLVQSGAE | 351 | DIQMTQSPSS |
| | | VKKPGASVKV | | LSASVGDRVT |
| | | SCKASGYTFT | | ITCRASQSIS |
| | | SHDIDWVRQA | | SYLNWYQQKP |
| | | PGQGLEWMGW | | GKAPKLLIYR |
| | | MNPNNGNTVY | | ASHLEGGVPS |
| | | AQRFQGRVTM | | RFSGSGSGTD |
| | | TRDTSTSTVY | | FTLTISSLQP |
| | | MELSSLRSED | | EDFATYYCQQ |
| | | TAVYYCARWK | | ADSLPLTFGQ |
| | | VYSGSYYGGA | | GTKVEIK |
| | | GYFDLWGRGT | | |
| | | LVTVSS | | |
| GITR-Mab-44 | 355 | QVQLVQSGAE | 359 | DIQMTQSPSS |
| | | VKKPGASVKV | | LSASVGDRVT |
| | | SCKASGYTFT | | ITCRASESIS |
| | | SHDIDWVRQA | | PWVAWYQQKP |
| | | PGQGLEWMGW | | GKAPKLLIYA |
| | | INPSGDSTSY | | ASTLQSGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCARYY | | GTSTPYTFGQ |
| | | GGNSYAFDIW | | GTKLEIK |
| | | GQGTMVTVSS | | |
| GITR-Mab-45 | 363 | QVQLVQSGAE | 367 | DIQMTQSPSS |
| | | VKKPGASVKV | | LSASVGDRVT |
| | | SCKASGYTFT | | ITCRASQGID |
| | | SYGISWVRQA | | NWLAWYQQKP |
| | | PGQGLEWMGW | | GKAPKLLIYG |
| | | MNPNSGNTGY | | ASTLQSGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCAREV | | SYTARFTFGP |
| | | YGDLDYWGQG | | GTKVDIK |
| | | TLVTVSS | | |
| GITR-Mab-46 | 371 | QVQLVQSGAE | 375 | DIQMTQSPSS |
| | | VKKPGASVKV | | LSASVGDRVT |
| | | SCKASGGSFS | | ITCQASQDIS |
| | | NYAINWVRQA | | NYLNWYQQKP |
| | | PGQGLEWMGW | | GKAPKLLIYE |
| | | MNPYSGNTGY | | ASVLESGVPS |
| | | AQKFQGRV | | RFSGSGS |
| | | TMTRDTSTST | | GTDFTLTISS |
| | | VYMELSSLRS | | LQPEDFATYY |
| | | EDTAVYYCAR | | CQQSYTTQYT |
| | | EITANYYYGM | | FGQGTKVEIK |
| | | DVWGQGTKVT | | |
| | | VSS | | |
| GITR-Mab-47 | 379 | QVQLVQSGAE | 383 | DIVMTQSPLS |
| | | VKKPGSSVKV | | LPVTPGEPAS |
| | | SCKASGGTFS | | ISCRSSQSLL |
| | | TYAINWVRQA | | HSNGYNYLDW |
| | | PGQGLEWMGI | | YLQKPGQSPQ |
| | | INPTDGDTSY | | LLIYLGSNRA |
| | | AQKFQGRVTI | | SGVPDRFSGS |
| | | TADESTSTAY | | GSGTDFTLKI |
| | | MELSSLRSED | | SRVEAEDVGV |
| | | TAVYYCARAA | | YYCMQALHIP |
| | | YYYYGMDVWG | | YTFGQGTKLE |
| | | QGTTVTVSS | | IK |
| GITR-Mab-48 | 387 | QVQLVQSGAE | 391 | DIQMTQSPSS |
| | | VKKPGASVKV | | LSASVGDRVT |
| | | SCKASGYSFT | | ITCRASQTIS |
| | | SHDIDWVRQA | | TYLNWYQQKP |
| | | PGQGLEWMGW | | GKAPKLLIYA |
| | | INPNSGGTNY | | ASSLQSGVPS |
| | | AQKFQGRVTM | | RFSGSGSGTD |
| | | TRDTSTSTVY | | FTLTISSLQP |
| | | MELSSLRSED | | EDFATYYCQQ |
| | | TAVYYCARIR | | SYSTPWTFGQ |
| | | GYYGSGSYHD | | GTKVEIK |
| | | AFDIWGQGTT | | |
| | | VTVSS | | |

TABLE 4
GITR Mab CDR Sequences

| | HCDR1 | | HCDR2 | | HCDR3 | | LCDR1 | | LCDR2 | | LCDR3 | |
|---|---|---|---|---|---|---|---|---|---|---|---|---|
| | SEQ | | SEQ | | SEQ | | SEQ | | SEQ | | SEQ | |
| Mab | ID | HCDR1 | ID | HCDR2 | ID | HCDR3 | ID | LCDR1 | ID | LCDR2 | ID | LCDR3 |
| GITR- | 2 | YTFT | 3 | GMIN | 4 | CARS | 6 | RASQ | 7 | GAST | 8 | CQQY |
| Mab-1 | | NYHL | | PNDG | | TYYY | | SVYS | | RAT | | HSYP |
| | | H | | STTY | | DSSG | | NYLA | | | | LTF |
| | | | | A | | YYYY | | | | | | |
| | | | | | | YYGM | | | | | | |
| | | | | | | DVW | | | | | | |
| GITR- | 10 | YRFT | 11 | GGII | 12 | CAYG | 14 | RSSQ | 15 | LGSY | 16 | CRQA |
| Mab-2 | | GYHL | | PIFG | | VPPD | | SLLH | | RAS | | LRTP |
| | | H | | TANY | | PW | | SNGY | | | | LTF |
| | | | | A | | | | NYLD | | | | |
| GITR- | 18 | YTFT | 19 | GWIS | 20 | CARS | 22 | RASQ | 23 | GAST | 24 | CQQY |
| Mab-3 | | SYDI | | GYNG | | HEYY | | SVSS | | RAT | | YTTP |
| | | N | | NTNY | | YYYG | | SYLA | | | | FTF |
| | | | | A | | MDVW | | | | | | |
| GITR- | 26 | FTFS | 27 | SAIS | 28 | CARD | 30 | RSSQ | 31 | LGSN | 32 | CMQA |
| Mab-4 | | SYT)MH | | SSGG | | SWVP | | SLLH | | RAS | | THSP |
| | | | | SIYY | | KGPN | | SNGY | | | | YTF |
| | | | | A | | RKYY | | TIYL | | | | |
| | | | | | | YYGM | | D | | | | |
| | | | | | | DVW | | | | | | |
| GITR- | 34 | FTFD | 35 | SAIG | 36 | CARD | 38 | RASQ | 39 | AASS | 40 | CQQS |
| Mab-5 | | AYAM | | TGGD | | LYGS | | GIKN | | LQS | | YSVP |
| | | H | | TYYA | | GSPQ | | DLG | | | | FTF |
| | | | | | | YYYY | | | | | | |
| | | | | | | YGMD | | | | | | |
| | | | | | | VW | | | | | | |
| GITR- | 42 | YAFT | 43 | GWMN | 44 | CARE | 46 | RSSQ | 47 | LGSR | 48 | CMQG |
| Mab-6 | | AYYL | | PNSG | | GWGY | | SLLH | | RAS | | SHWP |
| | | H | | NTGY | | YDGG | | SNGY | | | | PTF |
| | | | | A | | FDPW | | NYLD | | | | |
| GITR- | 50 | GTSS | 51 | GVIN | 52 | CARW | 54 | QASQ | 55 | AASS | 56 | CQQS |
| Mab-7 | | TYAV | | PNDG | | TPLF | | DISN | | LQS | | YSTP |
| | | T | | STTY | | GVVI | | YLN | | | | PTF |
| | | | | A | | PDYY | | | | | | |
| | | | | | | YYGM | | | | | | |
| | | | | | | DVW | | | | | | |
| GITR- | 58 | GTFS | 59 | GWMN | 60 | CITD | 62 | KSSQ | 63 | WAST | 64 | CQQY |
| Mab-8 | | SYVI | | PGSG | | SEDD | | SVLY | | RES | | YNTP |
| | | S | | NTGY | | YW | | SSNN | | | | YTF |
| | | | | A | | | | KNYL | | | | |
| | | | | | | | | A | | | | |
| GITR- | 66 | HTFT | 67 | GVIN | 68 | CARD | 70 | QASQ | 71 | GASN | 72 | CQQG |
| Mab-9 | | SQYM | | PNDG | | GAYY | | DIGN | | LET | | YSFP |
| | | H | | STSY | | YDSS | | YLN | | | | LTF |
| | | | | A | | GYYR | | | | | | |
| | | | | | | SSNF | | | | | | |
| | | | | | | DYW | | | | | | |
| GITR- | 74 | YTFT | 75 | GWMN | 76 | CARG | 78 | RASQ | 79 | AAST | 80 | CQQY |
| Mab- | | SNYM | | PNSG | | RGGQ | | GISS | | LQS | | NSYS |
| 10 | | H | | NTAY | | LLFD | | WLA | | | | TF |
| | | | | A | | YW | | | | | | |
| GITR- | 82 | GTFS | 83 | GUNP | 84 | CARW | 86 | RASQ | 87 | AASS | 88 | CQQS |
| Mab- | | NYGI | | TDGS | | WGSG | | SISS | | LES | | YSTP |
| 11 | | S | | TTYA | | WSWY | | YLN | | | | PTF |
| | | | | | | FDLW | | | | | | |
| GITR- | 90 | YTFT | 91 | GILS | 92 | CARG | 94 | RASQ | 95 | AASS | 96 | CQQS |
| Mab- | | SYAI | | PSGG | | PWYF | | GISN | | LQS | | YSTP |
| 12 | | N | | GTSY | | DLW | | SLA | | | | FTF |
| | | | | A | | | | | | | | |
| GITR- | 98 | GTFS | 99 | GGIV | 100 | CARG | 102 | KSSQ | 103 | WAST | 104 | CQQY |
| Mab- | | SYAI | | PMLG | | SWLV | | SLLY | | RES | | YSTP |
| 13 | | S | | SPHY | | ADFQ | | SSNN | | | | ITF |
| | | | | A | | HW | | RNYL | | | | |
| | | | | | | | | A | | | | |
| GITR- | 106 | FRFS | 107 | SGIS | 108 | CARV | 110 | QASQ | 111 | DASS | 112 | CQQA |
| Mab- | | VYWM | | GSGG | | RRDG | | DISN | | LEA | | NSFP |
| 14 | | S | | TTYY | | YNYN | | YLN | | | | PTF |
| | | | | A | | FDYW | | | | | | |
| GITR- | 114 | FTFT | 115 | GGII | 116 | CAKG | 118 | KSSQ | 119 | WASI | 120 | CQQY |
| Mab- | | TSAV | | PIFG | | SGYE | | SVLY | | RES | | YSTP |
| 15 | | Q | | TANY | | FPGG | | NSNN | | | | LTF |
| | | | | A | | SEYF | | LNYL | | | | |
| | | | | | | QHW | | A | | | | |
| GITR- | 122 | GTLS | 123 | GWID | 124 | CARO | 126 | RASR | 127 | GASS | 128 | CQQS |
| Mab- | | SYAI | | PNSG | | GLLW | | SISN | | LQS | | YSTL |
| 16 | | S | | GTNY | | FGES | | YLN | | | | VTF |
| | | | | A | | GSIY | | | | | | |
| | | | | | | YYYG | | | | | | |
| | | | | | | MDVW | | | | | | |
| GITR- | 130 | YTFT | 131 | GWMN | 132 | CARL | 134 | KSSQ | 135 | WAST | 136 | CQQY |
| Mab- | | SYGI | | PNSG | | GLWF | | SVLY | | REP | | YSTP |
| 17 | | S | | NTGY | | GEYQ | | SSNN | | | | LTF |
| | | | | A | | YYFD | | KNYL | | | | |
| | | | | | | YW | | A | | | | |
| GITR- | 138 | FTFS | 139 | SGIS | 140 | CAKL | 142 | RASE | 143 | AASS | 144 | CQQS |
| Mab- | | SYWM | | WNGG | | GIAV | | SIST | | LQS | | YTLP |
| 18 | | S | | TVGY | | KSHW | | WLA | | | | LTF |
| | | | | A | | YFDL | | | | | | |
| | | | | | | W | | | | | | |
| GITR- | 146 | YTFT | 147 | GIIN | 148 | CARA | 150 | RASQ | 151 | AASS | 152 | CQQY |
| Mab- | | SYDI | | PSGG | | SSGG | | DIVN | | LQS | | YSYP |
| 19 | | N | | STSY | | YYYY | | WLA | | | | LTF |
| | | | | A | | YGMD | | | | | | |
| | | | | | | VW | | | | | | |
| GITR- | 154 | GTFS | 155 | GVIN | 156 | CARD | 158 | RASQ | 159 | DASN | 160 | CQQS |
| Mab- | | NYAI | | PRGG | | YSIP | | SIST | | LET | | YSTP |
| 20 | | S | | STTY | | YYGM | | YLN | | | | LTF |
| | | | | A | | DVW | | | | | | |
| GITR- | 162 | FTFS | 163 | AVIA | 164 | CARV | 166 | QASQ | 167 | AASS | 168 | CQQS |
| Mab- | | NHYM | | LDGS | | GPGG | | DISN | | LQS | | YNSP |
| 21 | | S | | YRYY | | MDVR | | YLN | | | | RVYT |
| | | | | A | | | | | | | | F |
| GITR- | 170 | GRFS | 171 | GUNP | 172 | CARD | 174 | RASQ | 175 | AAST | 176 | CQQS |
| Mab- | | TYAL | | TDGS | | VYSS | | SISN | | LQS | | YSVP |
| 22 | | S | | TTYA | | SWYS | | WLA | | | | FTF |
| | | | | | | DYW | | | | | | |
| GITR- | 178 | FPFS | 179 | SAIS | 180 | CAGP | 182 | RASQ | 183 | AAST | 184 | CQQY |
| Mab- | | TYAI | | GSGG | | DWYF | | AISN | | LQR | | YSTP |
| 23 | | H | | STYY | | DLW | | SLA | | | | YTF |
| | | | | A | | | | | | | | |
| GITR- | 186 | GTFN | 187 | GTIN | 188 | CARV | 190 | RASQ | 191 | AASS | 192 | CQQA |
| Mab- | | NYAI | | PRDG | | GYYG | | NITN | | LQS | | YSFP |
| 24 | | N | | DTTY | | SGSY | | WLA | | | | WTF |
| | | | | A | | YSYY | | | | | | |
| | | | | | | GMDV | | | | | | |
| | | | | | | W | | | | | | |
| GITR- | 204 | GTFS | 205 | GWVS | 206 | CARD | 208 | RTSQ | 209 | AASS | 210 | CQQS |
| Mab- | | SYAI | | GYNG | | RVES | | SIRR | | LQS | | YSIP |
| 25 | | S | | NANY | | GYSY | | YLN | | | | WTF |
| | | | | A | | HDAF | | | | | | |
| | | | | | | DIW | | | | | | |
| GITR- | 212 | YTFT | 213 | GWMN | 214 | CAKD | 216 | QASQ | 217 | SASS | 218 | CQQS |
| Mab- | | SYGI | | PNNG | | SDWY | | SISR | | LQS | | YSTP |
| 26 | | S | | NTVY | | GAFD | | WLA | | | | WTF |
| | | | | A | | IW | | | | | | |
| GITR- | 220 | YTFT | 221 | GWMN | 222 | CARO | 224 | QASH | 225 | AASN | 226 | CQQA |
| Mab- | | SYDI | | TNSG | | AYSS | | DIDN | | LQS | | NSFP |
| 27 | | N | | DTGY | | SWYW | | YLN | | | | LTF |
| | | | | A | | YGWF | | | | | | |
| | | | | | | DPW | | | | | | |
| GITR- | 228 | YTFP | 229 | GWMN | 230 | CARG | 232 | RASQ | 233 | GAST | 234 | CQQY |
| Mab- | | NYGI | | PNSG | | MPGR | | SVSS | | RAT | | HTYP |
| 28 | | T | | YTGY | | GFDP | | NYLA | | | | PTF |
| | | | | A | | W | | | | | | |
| GITR- | 236 | YTFS | 237 | GRIN | 238 | CARE | 240 | RASQ | 241 | GASS | 242 | CQQS |
| Mab- | | SDYI | | PSGG | | RGAA | | SISS | | LQS | | YRTP |
| 29 | | H | | STLY | | DTWY | | YLN | | | | LTF |
| | | | | A | | FDLW | | | | | | |
| GITR- | 244 | GTFS | 245 | GIID | 246 | CARV | 248 | RASQ | 249 | AASS | 250 | CQQS |
| Mab- | | SYAI | | PTGG | | GYYY | | SVST | | LQS | | YSTP |
| 30 | | S | | ATAY | | GMDV | | YLN | | | | LTF |
| | | | | A | | W | | | | | | |
| GITR- | 252 | GTFS | 253 | GGII | 254 | CARS | 256 | RASQ | 257 | GAST | 258 | CQQY |
| Mab- | | SYAI | | PISS | | YDSR | | TVGS | | RAT | | YSTP |
| 31 | | S | | ATSI | | YYGM | | RYLA | | | | WTF |
| | | | | P | | DVW | | | | | | |
| GITR- | 260 | GTFS | 261 | GWMN | 262 | CARG | 264 | RVSQ | 265 | AASS | 266 | CQQS |
| Mab- | | SYAI | | PNSG | | PWYF | | GISN | | LQS | | YSTV |
| 32 | | S | | DTGY | | DLW | | SLA | | | | YTF |
| | | | | A | | | | | | | | |
| GITR- | 268 | FMFS | 269 | SYIS | 270 | CARR | 272 | RASQ | 273 | DASN | IIJ274 | CQQA |
| Mab- | | SYSM | | GNSG | | LHGM | | SIST | | LET | | YRFP |
| 33 | | N | | YTNY | | DVW | | WLA | | | | VAF |
| | | | | A | | | | | | | | |
| GITR- | 276 | FTFS | 277 | SVIS | 278 | CARE | 280 | RASQ | 281 | TAST | 282 | CQQT |
| Mab- | | SYWM | | NSGG | | GWGY | | DIRN | | LQS | | FRTP |
| 34 | | H | | ATYY | | GMDV | | YLA | | | | LTF |
| | | | | A | | W | | | | | | |
| GITR- | 284 | YTFT | 285 | GLIT | 286 | CARE | 288 | QASQ | 289 | AAST | 290 | CQQS |
| Mab- | | NYYM | | FSGG | | MEYS | | GISS | | L | | YSTP |
| 35 | | H | | RTKY | | SSWY | | YLN | | | | LTF |
| | | | | A | | WTDP | | | | | | |
| | | | | | | W | | | | | | |
| GITR- | 292 | YTFT | 293 | GWIN | 294 | CARE | 296 | RASQ | 297 | AASS | 298 | CQQS |
| Mab- | | RNYI | | PKSG | | SGWA | | SISS | | LNS | | YSTP |
| 36 | | H | | GTKY | | TEYW | | YLN | | | | LTF |
| | | | | A | | YFDL | | | | | | |
| | | | | | | W | | | | | | |
| GITR- | 300 | YTFT | 301 | GWIN | 302 | CASP | 304 | KSSQ | 305 | WAST | 306 | CQQY |
| Mab- | | SYYM | | PNSG | | GDYC | | SIFY | | RES | | YSTP |
| 37 | | H | | GTNY | | SGGS | | SSNS | | | | LTF |
| | | | | A | | CYSD | | KNYL | | | | |
| | | | | | | DAFD | | A | | | | |
| | | | | | | IW | | | | | | |
| GITR- | 308 | GTFR | 309 | GWMN | 310 | CARE | 312 | RASQ | 313 | SASN | 314 | CQQG |
| Mab- | | NYAI | | PNSG | | DVDT | | GISN | | L | | YSSR |
| 38 | | N | | NTGY | | ASQA | | YLA | | | | YTF |
| | | | | A | | YFDY | | | | | | |
| | | | | | | W | | | | | | |
| GITR- | 316 | FTFS | 317 | SSIS | 318 | CARD | 320 | RASQ | 321 | EASK | 322 | CQQS |
| Mab- | | SYWM | | WSST | | GQLG | | SISD | | LAT | | YTTP |
| 39 | | S | | YIYY | | HWHF | | WLA | | | | LTF |
| | | | | A | | DLW | | | | | | |
| GITR- | 324 | FTFR | 325 | SGIS | 326 | CARE | 328 | RSSQ | 329 | LGSN | 330 | CMQG |
| Mab- | | NHWM | | WNSG | | EYAT | | SLLH | | RAS | | THWP |
| 40 | | S | | SIDY | | FDYW | | SNGY | | | | PTF |
| | | | | A | | | | NYLD | | | | |
| GITR- | 332 | GSFS | 333 | GWMN | 334 | CARE | 336 | RASQ | 337 | AASS | 338 | CQQS |
| Mab- | | DYAV | | PNSG | | LVRD | | DIRN | | L | | FSTF |
| 41 | | S | | NTGY | | GYNF | | DLA | | | | YTF |
| | | | | A | | ALDY | | | | | | |
| | | | | | | W | | | | | | |
| GITR- | 340 | YIFT | 341 | GWIN | 342 | CARG | 344 | RSSQ | 345 | LGSN | 346 | CMQA |
| Mab- | | NYWI | | PHSG | | LGSA | | SLLH | | RAS | | LQTP |
| 42 | | Q | | ATKY | | FDIW | | SNGY | | | | LTF |
| | | | | A | | | | NYLD | | | | |
| GITR- | 348 | YTFT | 349 | GWMN | 350 | CARW | 352 | RASQ | 353 | RASH | 354 | CQQA |
| Mab- | | SHDI | | PNNG | | YWSG | | SISS | | LEG | | DSLP |
| 43 | | D | | NTVY | | SYYG | | YLN | | | | LTF |
| | | | | A | | GAGY | | | | | | |
| | | | | | | FDLW | | | | | | |
| GITR- | 356 | YTFT | 357 | GWIN | 358 | CARY | 360 | RASE | 361 | AAST | 362 | CQQG |
| Mab- | | SHDI | | PSGD | | YGGN | | SISP | | LQS | | TSTP |
| 44 | | D | | STSY | | SYAF | | WVA | | | | YTF |
| | | | | A | | DIW | | | | | | |
| GITR- | 364 | YTFT | 365 | GWMN | 366 | CARE | 368 | RASQ | 369 | GAST | 370 | CQQS |
| Mab- | | SYGI | | PNSG | | VYGD | | GIDN | | LQS | | YTAR |
| 45 | | S | | NTGY | | LDYW | | WLA | | | | FTF |
| | | | | A | | | | | | | | |
| GITR- | 372 | GSFS | 373 | GWMN | 374 | CARE | 376 | QASQ | 377 | EASV | 378 | CQQS |
| Mab- | | NYAI | | PYSG | | ITAN | | DISN | | LES | | YTTQ |
| 46 | | N | | NTGY | | YYYG | | YLN | | | | YTF |
| | | | | A | | MDVW | | | | | | |
| GITR- | 380 | GTFS | 381 | GIIN | 382 | CARA | 384 | RSSQ | 385 | LGSN | 386 | CMQA |
| Mab- | | TYAI | | PTDG | | AYYY | | SLLH | | RAS | | LHIP |
| 47 | | N | | DTSY | | YGMD | | SNGY | | | | YTF |
| | | | | A | | VW | | NYLD | | | | |
| GITR- | 388 | YSFT | 389 | GWIN | 390 | CARI | 392 | RASQ | 393 | AASS | 394 | CQQS |
| Mab- | | SHDI | | PNSG | | RGYY | | TIST | | LQS | | YSTP |
| 48 | | D | | GTNY | | GSGS | | YLN | | | | WTF |
| | | | | A | | YHDA | | | | | | |
| | | | | | | FDIW | | | | | | |

The VH and VL sequences in Table 3 were inserted into proprietary IgM and light chain vectors by standard cloning methods. Mammalian cells were then co-transfected with IgM heavy chain and light chain vectors, along with a vector encoding a variant J-chain with a mutation to increase half-life (Y102A or J*, see PCT Appl. No. PCT/US2019/20374, which is incorporated herein by reference in its entirety) by standard procedures. Anti-GITR IgMs with the variant J chain J*is purified using the Capture Select IgM affinity matrix (BAC, Thermo Fisher Catalog #2890.05) according to manufacturer's recommendation. The VH and VL of GITR-Mab-3, GITR-Mab-6, GITR-Mab-11, and GITR-Mab 14 were also inserted into IgG and light chain vectors and expressed in mammalian cells. Human anti-GITR IgM with J chain was purified from culture supernatants using the Capture Select IgM affinity matrix (BAC, Thermo Fisher Catalog #2890.05) according to manufacturer's recommendation. Human anti-GITR IgGs were purified from culture supernatants using the MabSelectSuRe affinity matrix (GE Life Sciences Catalog #17-5438-01) according to manufacturer's recommendation. Human anti-GITR IgM with or without J chain is purified using the Capture Select IgM affinity matrix (BAC, Thermo Fisher Catalog #2890.05) according to manufacturer's recommendation. The IgG and IgM/J* versions of GITR-Mab-3, GITR-Mab-6, GITR-Mab-11, and GITR-Mab 14 and others were then subjected additional testing as outlined below.

### Example 2

### Characterization of Anti-GITR Mabs

### GITR Binding Assay

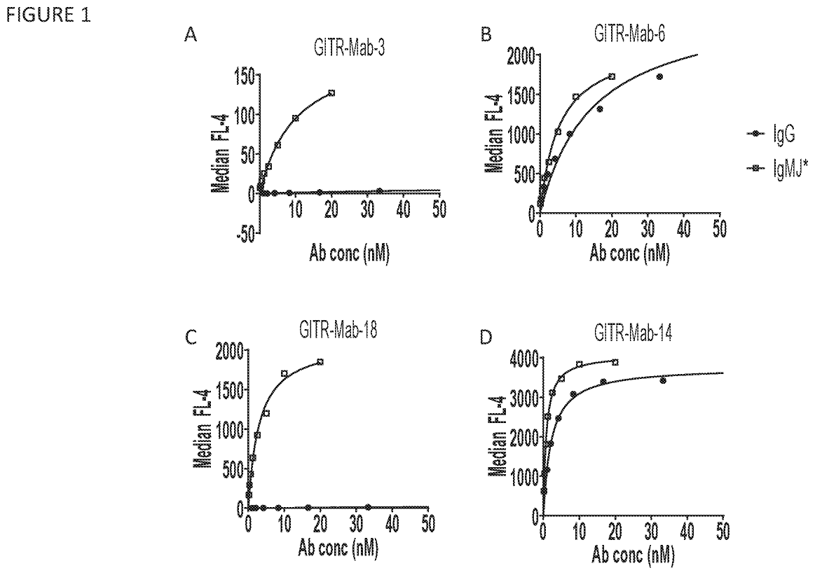

IgG and IgM/J versions of GITR-Mab-3, GITR-Mab-6, GITR-Mab-11, and GITR-Mab 14 were tested for binding to GITR-expressing 293 cells by flow cytometry by the following method. HEK 293 cells expressing human GITR (CrownBio, #C2011) and negative control Expi293 cells (10,000 cells/well) were stained with serial dilutions of IgG and IgMJ* versions of GITR-Mab-3, GITR-Mab-6, GITR-Mab-11, and GITR-Mab 14 for 30 minutes at 4° C. Cells were washed twice, then stained for 30 minutes at 4° C. with 5 ug/mL mouse anti-human kappa-ALEXA FLUOR® 647 secondary antibody (Southern Biotech #9230-31) diluted in FACS buffer. Cells were washed twice, resuspended in FACS Stain Buffer, and signal was acquired by flow cytometry. The results are shown in FIG. 1A-1D. All of the IgM antibodies bound to GITR-expressing cells. The IgG versions of GITR-Mab-6 and GITR-Mab-14 likewise bound to the GITR-expressing cells.

### GITR Activation Assay

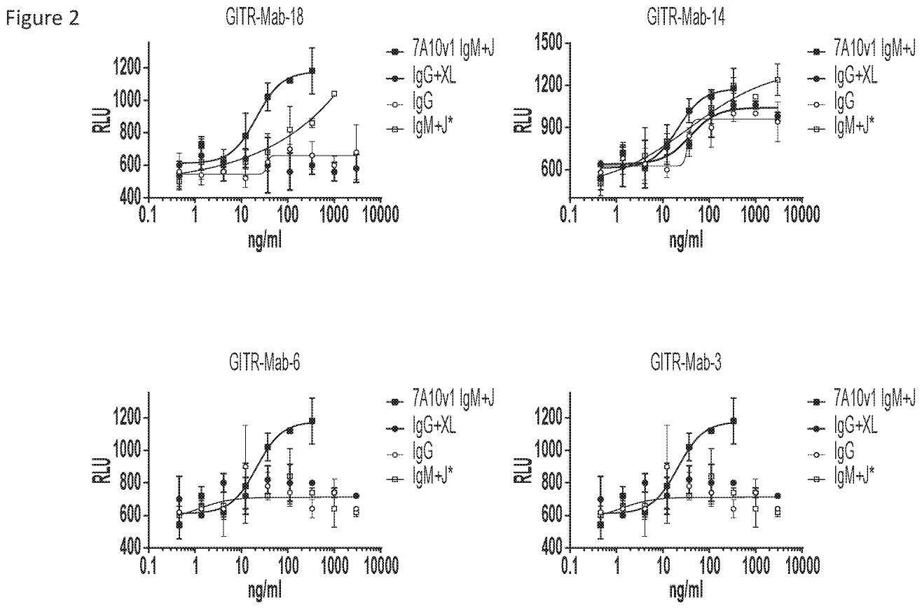

Activity of the IgG and IgM versions of GITR-Mab-11 and GITR-Mab-14 were tested by the following reporter assay. The reporter activity for anti GITR Mabs was measured using the GITR/NFkB-luciferase reporter Jurkat cell line (BPS Biosciences #60546) according to manufacturer's protocol. The cells were plated at 20000 cells/well and incubated with serial dilutions of IgG (with and without 10 μg/mL plate-bound anti-human IgG Fc crosslinker (Biolegend #409302) and IgMJ* versions of GITR-Mab-11 and GITR-Mab-14, for 6 hours at 37° C. Anti-GITR Mab #1 IgM+wild-type J chain (see PCT Publication No. WO/2018/017889, which is incorporated herein by reference in its entirety) was used as a positive control. Bio-Glo reagent was added and after 10 minutes luminescence was read on a plate reader. The results are shown in FIG. 2A-B. Both IgM antibodies exhibited increased agonist activity relative to the IgG versions of the antibodies.

The above protocol was repeated for additional IgM/J* versions of various GITR-Mabs. The results are shown in Table 5.

TABLE 5
GITR Activation Assay

| Antibody | Max Fold activation | EC50 (ng/ml) |
|---|---|---|
| GITR-Mab-3 IgM/J* | 1.3 | 5000 |
| GITR-Mab-7 IgM/J* | 1 | 5000 |
| GITR-Mab-12 IgM/J* | 2.1 | 169.7 |
| GITR-Mab-14 IgM/J* | 1.6 | 339.8 |
| GITR-Mab-22 IgM/J* | 2.2 | 1184 |
| GITR-Mab-23 IgM/J* | 2.4 | 172.9 |
| GITR-Mab-25 IgM/J* | 3.3 | 20.9 |
| GITR-Mab-26 IgM/J* | 2.8 | 18.8 |
| GITR-Mab-27 IgM/J* | 2.9 | 94 |
| GITR-Mab-28 IgM/J* | 4.7 | 78.7 |
| GITR-Mab-29 IgM/J* | 3.7 | 970 |
| GITR-Mab-31 IgM/J* | 3 | 45 |
| GITR-Mab-32 IgM/J* | 2.3 | 641 |
| GITR-Mab-33 IgM/J* | 3.5 | 75 |
| GITR-Mab-34 IgM/J* | 1.8 | 5000 |
| GITR-Mab-35 IgM/J* | 5.4 | 87 |
| GITR-Mab-36 IgM/J* | 6.2 | 1563 |
| GITR-Mab-38 IgM/J* | 1 | 5000 |
| GITR-Mab-40 IgM/J* | 1 | 5000 |

### Cytokine Production Assay

The ability of the GITR-Mabs to enhance T cell activation was assessed as follows. 96-well tissue culture plates were coated with 3 μg/mL of anti-CD3 (clone OKT3, eBioscience #16-0037-85) at 4° C. overnight, and then washed 2 times with PBS. For IgG crosslinking, wells were additionally coated with 10 μg/mL of anti-human IgG Fc crosslinker (Biolegend #409302).

Purified human CD4 T cells (Astarte Biologics) were then seeded at 0.2×10⁶ cells/well on the coated 96-well plates. 200 ng/mL of soluble GITR-Mabs IgM or IgG were added to the purified human CD4 T cells for 3 days at 37° C. and 5% CO₂.

Alternatively, PBMCs were incubated with 1000 ng/ml, 200 ng/ml and 40 ng/mL of anti GITR IgM or IgG antibodies for 5 days at 37 ° C.

Supernatants were assayed for a panel of cytokines including IFNγ, IL-4, TNF, IL-10, and IL-6 by cytometric bead array (CBA) according to manufacturer's protocol (BD #551809). The antibodies had little effect on cytokine production.

The breadth and scope of the present disclosure should not be limited by any of the above-described exemplary embodiments but should be defined only in accordance with the following claims and their equivalents.

What is claimed is:

1. An antibody or antigen-binding fragment or derivative thereof comprising an antigen-binding domain that specifically binds to glucocorticoid-induced TNF receptor (GITR), wherein the antigen-binding domain comprises a heavy chain variable region (VH) and light chain variable region (VL), wherein the VH and VL comprise six immunoglobulin complementarity determining regions HCDR1, HCDR2, HCDR3, LCDR1, LCDR2, and LCDR3,
wherein the HCDR1, HCDR2, HCDR3, LCDR1, LCDR2, and LCDR3 comprise, respectively, the amino acid sequences SEQ ID NO: 2, SEQ ID NO: 3, SEQ ID NO: 4, SEQ ID NO: 6, SEQ ID NO: 7, and SEQ ID NO: 8; SEQ ID NO: 10, SEQ ID NO: 11, SEQ ID NO: 12, SEQ ID NO: 14, SEQ ID NO: 15, and SEQ ID NO: 16; SEQ ID NO: 18, SEQ ID NO: 19, SEQ ID NO: 20, SEQ ID NO: 22, SEQ ID NO: 23, and SEQ ID NO: 24; SEQ ID NO: 26, SEQ ID NO: 27, SEQ ID NO: 28, SEQ ID NO: 30, SEQ ID NO: 31, and SEQ ID NO: 32; SEQ ID NO: 34, SEQ ID NO: 35, SEQ ID NO: 36, SEQ ID NO: 38, SEQ ID NO: 39, and SEQ ID NO: 40; SEQ ID NO: 42, SEQ ID NO: 43, SEQ ID NO: 44, SEQ ID NO: 46, SEQ ID NO: 47, and SEQ ID NO: 48; SEQ ID NO: 50, SEQ ID NO: 51, SEQ ID NO: 52, SEQ ID NO: 54, SEQ ID NO: 55, and SEQ ID NO: 56; SEQ ID NO: 58, SEQ ID NO: 59, SEQ ID NO: 60, SEQ ID NO: 62, SEQ ID NO: 63, and SEQ ID NO: 64; SEQ ID NO: 66, SEQ ID NO: 67, SEQ ID NO: 68, SEQ ID NO: 70, SEQ ID NO: 71, and SEQ ID NO: 72; SEQ ID NO: 74, SEQ ID NO: 75, SEQ ID NO: 76, SEQ ID NO: 78, SEQ ID NO: 79, and SEQ ID NO: 80; SEQ ID NO: 82, SEQ ID NO: 83, SEQ ID NO: 84, SEQ ID NO: 86, SEQ ID NO: 87, and SEQ ID NO: 88; SEQ ID NO: 90, SEQ ID NO: 91, SEQ ID NO: 92, SEQ ID NO: 94, SEQ ID NO: 95, and SEQ ID NO: 96; SEQ ID NO: 98, SEQ ID NO: 99, SEQ ID NO: 100, SEQ ID NO: 102, SEQ ID NO: 103, and SEQ ID NO: 104; SEQ ID NO: 106, SEQ ID NO: 107, SEQ ID NO: 108, SEQ ID NO: 110, SEQ ID NO: 111, and SEQ ID NO: 112; SEQ ID NO: 114, SEQ ID NO: 115, SEQ ID NO: 116, SEQ ID NO: 118, SEQ ID NO: 119, and SEQ ID NO: 120; SEQ ID NO: 122, SEQ ID NO: 123, SEQ ID NO: 124, SEQ ID NO: 126, SEQ ID NO: 127, and SEQ ID NO: 128; SEQ ID NO: 130, SEQ ID NO: 131, SEQ ID NO: 132, SEQ ID NO: 134, SEQ ID NO: 135, and SEQ ID NO: 136; SEQ ID NO: 138, SEQ ID NO: 139, SEQ ID NO: 140, SEQ ID NO: 142, SEQ ID NO: 143, and SEQ ID NO: 144; SEQ ID NO: 146, SEQ ID NO: 147, SEQ ID NO: 148, SEQ ID NO: 150, SEQ ID NO: 151, and SEQ ID NO: 152; SEQ ID NO: 154, SEQ ID NO: 155, SEQ ID NO: 156, SEQ ID NO: 158, SEQ ID NO: 159, and SEQ ID NO: 160; SEQ ID NO: 162, SEQ ID NO: 163, SEQ ID NO: 164, SEQ ID NO: 166, SEQ ID NO: 167, and SEQ ID NO: 168; SEQ ID NO: 170, SEQ ID NO: 171, SEQ ID NO: 172, SEQ ID NO: 174, SEQ ID NO: 175, and SEQ ID NO: 176; SEQ ID NO: 178, SEQ ID NO: 179, SEQ ID NO: 180, SEQ ID NO: 182, SEQ ID NO: 183, and SEQ ID NO: 184; SEQ ID NO: 186, SEQ ID NO: 187, SEQ ID NO: 188, SEQ ID NO: 190, SEQ ID NO: 191, and SEQ ID NO: 192; SEQ ID NO: 204, SEQ ID NO: 205, SEQ ID NO: 206, SEQ ID NO: 208, SEQ ID NO: 209, and SEQ ID NO: 210; SEQ ID NO: 212, SEQ ID NO: 213, SEQ ID NO: 214, SEQ ID NO: 216, SEQ ID NO: 217, and SEQ ID NO: 218; SEQ ID NO: 220, SEQ ID NO: 221, SEQ ID NO: 222, SEQ ID NO: 224, SEQ ID NO: 225, and SEQ ID NO: 226; SEQ ID NO: 228, SEQ ID NO: 229, SEQ ID NO: 230, SEQ ID NO: 232, SEQ ID NO: 233, and SEQ ID NO: 234; SEQ ID NO: 236, SEQ ID NO: 237, SEQ ID NO: 238, SEQ ID NO: 240, SEQ ID NO: 241, and SEQ ID NO: 242; SEQ ID NO: 244, SEQ ID NO: 245, SEQ ID NO: 246, SEQ ID NO: 248, SEQ ID NO: 249, and SEQ ID NO: 250; SEQ ID NO: 252, SEQ ID NO: 253, SEQ ID NO: 254, SEQ ID NO: 256, SEQ ID NO: 257, and SEQ ID NO: 258; SEQ ID NO: 260, SEQ ID NO: 261, SEQ ID NO: 262, SEQ ID NO: 264, SEQ ID NO: 265, and SEQ ID NO: 266; SEQ ID NO: 268, SEQ ID NO: 269, SEQ ID NO: 270, SEQ ID NO: 272, SEQ ID NO: 273, and SEQ ID NO: 274; SEQ ID NO: 276, SEQ ID NO: 277, SEQ ID NO: 278, SEQ ID NO: 280, SEQ ID NO: 281, and SEQ ID NO: 282; SEQ ID NO: 284, SEQ ID NO: 285, SEQ ID NO: 286, SEQ ID NO: 288, SEQ ID NO: 289, and SEQ ID NO: 290; SEQ ID NO: 292, SEQ ID NO: 293, SEQ ID NO: 294, SEQ ID NO: 296, SEQ ID NO: 297, and SEQ ID NO: 298; SEQ ID NO: 300, SEQ ID NO: 301, SEQ ID NO: 302, SEQ ID NO: 304, SEQ ID NO: 305, and SEQ ID NO: 306; SEQ ID NO: 308, SEQ ID NO: 309, SEQ ID NO: 310, SEQ ID NO: 312, SEQ ID NO: 313, and SEQ ID NO: 314; SEQ ID NO: 316, SEQ ID NO: 317, SEQ ID NO: 318, SEQ ID NO: 320, SEQ ID NO: 321, and SEQ ID NO: 322; SEQ ID NO: 324, SEQ ID NO: 325, SEQ ID NO: 326, SEQ ID NO: 328, SEQ ID NO: 329, and SEQ ID NO: 330; SEQ ID NO: 332, SEQ ID NO: 333, SEQ ID NO: 334, SEQ ID NO: 336, SEQ ID NO: 337, and SEQ ID NO: 338; SEQ ID NO: 340, SEQ ID NO: 341, SEQ ID NO: 342, SEQ ID NO: 344, SEQ ID NO: 345, and SEQ ID NO: 346; SEQ ID NO: 348, SEQ ID NO: 349, SEQ ID NO: 350, SEQ ID NO: 352, SEQ ID NO: 353, and SEQ ID NO: 354; SEQ ID NO: 356, SEQ ID NO: 357, SEQ ID NO: 358, SEQ ID NO: 360, SEQ ID NO: 361, and SEQ ID NO: 362; SEQ ID NO: 364, SEQ ID NO: 365, SEQ ID NO: 366, SEQ ID NO: 368, SEQ ID NO: 369, and SEQ ID NO: 370; SEQ ID NO: 372, SEQ ID NO: 373, SEQ ID NO: 374, SEQ ID NO: 376, SEQ ID NO: 377, and SEQ ID NO: 378; SEQ ID NO: 380, SEQ ID NO: 381, SEQ ID NO: 382, SEQ ID NO: 384, SEQ ID NO: 385, and SEQ ID NO: 386; or SEQ ID NO: 388, SEQ ID NO: 389, SEQ ID NO: 390, SEQ ID NO: 392, SEQ ID NO: 393, and SEQ ID NO: 394; or
wherein the HCDR1, HCDR2, HCDR3, LCDR1, LCDR2, and LCDR3 comprise, respectively, the amino acid sequences SEQ ID NO: 2, SEQ ID NO: 3, SEQ ID NO: 4, SEQ ID NO: 6, SEQ ID NO: 7, and SEQ ID NO: 8; SEQ ID NO: 10, SEQ ID NO: 11, SEQ ID NO: 12, SEQ ID NO: 14, SEQ ID NO: 15, and SEQ ID NO: 16; SEQ ID NO: 18, SEQ ID NO: 19, SEQ ID NO: 20, SEQ ID NO: 22, SEQ ID NO: 23, and SEQ ID NO: 24; SEQ ID NO: 26, SEQ ID NO: 27, SEQ ID NO: 28, SEQ ID NO: 30, SEQ ID NO: 31, and SEQ ID NO: 32; SEQ ID NO: 34, SEQ ID NO: 35, SEQ ID NO: 36, SEQ ID NO: 38, SEQ ID NO: 39, and SEQ ID NO: 40; SEQ ID NO: 42, SEQ ID NO: 43, SEQ ID NO: 44, SEQ ID NO: 46, SEQ ID NO: 47, and SEQ ID NO: 48; SEQ ID NO: 50, SEQ ID NO: 51, SEQ ID NO: 52, SEQ ID NO: 54, SEQ ID NO: 55, and SEQ ID NO: 56; SEQ ID NO: 58, SEQ ID NO: 59, SEQ ID NO: 60, SEQ ID NO: 62, SEQ ID NO: 63, and SEQ ID NO: 64; SEQ ID NO: 66, SEQ ID NO: 67, SEQ ID NO: 68, SEQ ID NO: 70, SEQ ID NO: 71, and SEQ ID NO: 72; SEQ ID NO: 74, SEQ ID NO: 75, SEQ ID NO: 76, SEQ ID NO: 78, SEQ ID NO: 79, and SEQ ID NO: 80; SEQ ID NO: 82, SEQ ID NO: 83, SEQ ID NO: 84, SEQ ID NO: 86, SEQ ID NO: 87, and SEQ ID NO: 88; SEQ ID NO: 90, SEQ ID NO: 91, SEQ ID NO: 92, SEQ ID NO: 94, SEQ ID NO: 95, and SEQ ID NO: 96; SEQ ID NO: 98, SEQ ID NO: 99, SEQ ID NO: 100, SEQ ID NO: 102, SEQ ID NO: 103, and SEQ ID NO: 104; SEQ ID NO: 106, SEQ ID NO: 107, SEQ ID NO: 108, SEQ ID NO: 110, SEQ ID NO: 111, and SEQ ID NO: 112; SEQ ID NO: 114, SEQ ID NO: 115, SEQ ID NO: 116, SEQ ID NO: 118, SEQ ID NO: 119, and SEQ ID NO: 120; SEQ ID NO: 122, SEQ ID NO: 123, SEQ ID NO: 124, SEQ ID NO: 126, SEQ ID NO: 127, and SEQ ID NO: 128; SEQ ID NO: 130, SEQ ID NO: 131, SEQ ID NO: 132, SEQ ID NO: 134, SEQ ID NO: 135, and SEQ ID NO: 136; SEQ ID NO: 138, SEQ ID NO: 139, SEQ ID NO: 140, SEQ ID NO: 142, SEQ ID NO: 143, and SEQ ID NO: 144; SEQ ID NO: 146, SEQ ID NO: 147, SEQ ID NO: 148, SEQ ID NO: 150, SEQ ID NO: 151, and SEQ ID NO: 152; SEQ ID NO: 154, SEQ ID NO: 155, SEQ ID NO: 156, SEQ ID NO: 158, SEQ ID NO: 159, and SEQ ID NO: 160; SEQ ID NO: 162, SEQ ID NO: 163, SEQ ID NO: 164, SEQ ID NO: 166, SEQ ID NO: 167, and SEQ ID NO: 168; SEQ ID NO: 170, SEQ ID NO: 171, SEQ ID NO: 172, SEQ ID NO: 174, SEQ ID NO: 175, and SEQ ID NO: 176; SEQ ID NO: 178, SEQ ID NO: 179, SEQ ID NO: 180, SEQ ID NO: 182, SEQ ID NO: 183, and SEQ ID NO: 184; SEQ ID NO: 186, SEQ ID NO: 187, SEQ ID NO: 188, SEQ ID NO: 190, SEQ ID NO: 191, and SEQ ID NO: 192; SEQ ID NO: 204, SEQ ID NO: 205, SEQ ID NO: 206, SEQ ID NO: 208, SEQ ID NO: 209, and SEQ ID NO: 210; SEQ ID NO: 212, SEQ ID NO: 213, SEQ ID NO: 214, SEQ ID NO: 216, SEQ ID NO: 217, and SEQ ID NO: 218 SEQ ID NO: 220, SEQ ID NO: 221, SEQ ID NO: 222, SEQ ID NO: 224, SEQ ID NO: 225, and SEQ ID NO: 226; SEQ ID NO: 228, SEQ ID NO: 229, SEQ ID NO: 230, SEQ ID NO: 232, SEQ ID NO: 233, and SEQ ID NO: 234; SEQ ID NO: 236, SEQ ID NO: 237, SEQ ID NO: 238, SEQ ID NO: 240, SEQ ID NO: 241, and SEQ ID NO: 242; SEQ ID NO: 244, SEQ ID NO: 245, SEQ ID NO: 246, SEQ ID NO: 248, SEQ ID NO: 249, and SEQ ID NO: 250; SEQ ID NO: 252, SEQ ID NO: 253, SEQ ID NO: 254, SEQ ID NO: 256, SEQ ID NO: 257, and SEQ ID NO: 258; SEQ ID NO: 260, SEQ ID NO: 261, SEQ ID NO: 262, SEQ ID NO: 264, SEQ ID NO: 265, and SEQ ID NO: 266; SEQ ID NO: 268, SEQ ID NO: 269, SEQ ID NO: 270, SEQ ID NO: 272, SEQ ID NO: 273, and SEQ ID NO: 274; SEQ ID NO: 276, SEQ ID NO: 277, SEQ ID NO: 278, SEQ ID NO: 280, SEQ ID NO: 281, and SEQ ID NO: 282; SEQ ID NO: 284, SEQ ID NO: 285, SEQ ID NO: 286, SEQ ID NO: 288, SEQ ID NO: 289, and SEQ ID NO: 290; SEQ ID NO: 292, SEQ ID NO: 293, SEQ ID NO: 294, SEQ ID NO: 296, SEQ ID NO: 297, and SEQ ID NO: 298; SEQ ID NO: 300, SEQ ID NO: 301, SEQ ID NO: 302, SEQ ID NO: 304, SEQ ID NO: 305, and SEQ ID NO: 306; SEQ ID NO: 308, SEQ ID NO: 309, SEQ ID NO: 310, SEQ ID NO: 312, SEQ ID NO: 313, and SEQ ID NO: 314; SEQ ID NO: 316, SEQ ID NO: 317, SEQ ID NO: 318, SEQ ID NO: 320, SEQ ID NO: 321, and SEQ ID NO: 322; SEQ ID NO: 324, SEQ ID NO: 325, SEQ ID NO: 326, SEQ ID NO: 328, SEQ ID NO: 329, and SEQ ID NO: 330; SEQ ID NO: 332, SEQ ID NO: 333, SEQ ID NO: 334, SEQ ID NO: 336, SEQ ID NO: 337, and SEQ ID NO: 338; SEQ ID NO: 340, SEQ ID NO: 341, SEQ ID NO: 342, SEQ ID NO: 344, SEQ ID NO: 345, and SEQ ID NO: 346; SEQ ID NO: 348, SEQ ID NO: 349, SEQ ID NO: 350, SEQ ID NO: 352, SEQ ID NO: 353, and SEQ ID NO: 354; SEQ ID NO: 356, SEQ ID NO: 357, SEQ ID NO: 358, SEQ ID NO: 360, SEQ ID NO: 361, and SEQ ID NO: 362; SEQ ID NO: 364, SEQ ID NO: 365, SEQ ID NO: 366, SEQ ID NO: 368, SEQ ID NO: 369, and SEQ ID NO: 370; SEQ ID NO: 372, SEQ ID NO: 373, SEQ ID NO: 374, SEQ ID NO: 376, SEQ ID NO: 377, and SEQ ID NO: 378; SEQ ID NO: 380, SEQ ID NO: 381, SEQ ID NO: 382, SEQ ID NO: 384, SEQ ID NO: 385, and SEQ ID NO: 386; or SEQ ID NO: 388, SEQ ID NO: 389, SEQ ID NO: 390, SEQ ID NO: 392, SEQ ID NO: 393, and SEQ ID NO: 394, except for one or two amino acid substitutions in one or more of the CDRs.

2. The antibody or fragment or derivative thereof of claim 1, wherein the VH further comprises framework regions (HFWs) HFW1, HFW2, HFW3, and HFW4, and wherein the VL further comprises framework regions (LFWs) LFW1, LFW2, LFW3, and LFW4.

3. The antibody or fragment or derivative thereof of claim 2, wherein the framework regions are derived from a human antibody.

4. The antibody or fragment or derivative thereof of claim 2, wherein the framework regions are derived from a non-human antibody.

5. The antibody or fragment or derivative thereof of any one of claims 1 to 3, wherein the VH comprises the amino acid sequence SEQ ID NO: 1, SEQ ID NO: 9, SEQ ID NO: 17, SEQ ID NO: 25, SEQ ID NO: 33, SEQ ID NO: 41, SEQ ID NO: 49, SEQ ID NO: 57, SEQ ID NO: 65, SEQ ID NO: 73, SEQ ID NO: 81, SEQ ID NO: 89, SEQ ID NO: 97, SEQ ID NO: 105, SEQ ID NO: 113, SEQ ID NO: 121, SEQ ID NO: 129, SEQ ID NO: 137, SEQ ID NO: 145, SEQ ID NO: 153, SEQ ID NO: 161, SEQ ID NO: 169, SEQ ID NO: 177, SEQ ID NO: 185, SEQ ID NO: 203, SEQ ID NO: 211, SEQ ID NO: 219, SEQ ID NO: 227, SEQ ID NO: 235, SEQ ID NO: 243, SEQ ID NO: 251, SEQ ID NO: 259, SEQ ID NO: 267, SEQ ID NO: 275, SEQ ID NO: 283, SEQ ID NO: 291, SEQ ID NO: 299, SEQ ID NO: 307, SEQ ID NO: 315, SEQ ID NO: 323, SEQ ID NO: 331, SEQ ID NO: 339, SEQ ID NO: 347, SEQ ID NO: 355, SEQ ID NO: 363, SEQ ID NO: 371, SEQ ID NO: 379, or SEQ ID NO: 387.

6. The antibody or fragment or derivative thereof of any one of claims 1 to 3, wherein the VL comprises the amino acid sequence SEQ ID NO: 5, SEQ ID NO: 13, SEQ ID NO: 21, SEQ ID NO: 29, SEQ ID NO: 37, SEQ ID NO: 45, SEQ ID NO: 53, SEQ ID NO: 61, SEQ ID NO: 69, SEQ ID NO: 77, SEQ ID NO: 85, SEQ ID NO: 93, SEQ ID NO: 101, SEQ ID NO: 109, SEQ ID NO: 117, SEQ ID NO: 125, SEQ ID NO: 133, SEQ ID NO: 141, SEQ ID NO: 149, SEQ ID NO: 157, SEQ ID NO: 165, SEQ ID NO: 173, SEQ ID NO: 181, SEQ ID NO: 189, SEQ ID NO: 207, SEQ ID NO: 215, SEQ ID NO: 223, SEQ ID NO: 231, SEQ ID NO: 239, SEQ ID NO: 247, SEQ ID NO: 255, SEQ ID NO: 263, SEQ ID NO: 271, SEQ ID NO: 279, SEQ ID NO: 287, SEQ ID NO: 295, SEQ ID NO: 303, SEQ ID NO: 311, SEQ ID NO: 319, SEQ ID NO: 327, SEQ ID NO: 335, SEQ ID NO: 343, SEQ ID NO: 351, SEQ ID NO: 359, SEQ ID NO: 367, SEQ ID NO: 375, SEQ ID NO: 383, or SEQ ID NO: 391.

7. The antibody or fragment or derivative thereof of any one of claims 1 to 3, wherein the VH and VL comprise, respectively, the amino acid sequences SEQ ID NO: 1 and SEQ ID NO: 5, SEQ ID NO: 9 and SEQ ID NO: 13, SEQ ID NO: 17 and SEQ ID NO: 21, SEQ ID NO: 25 and SEQ ID NO: 29, SEQ ID NO: 33 and SEQ ID NO: 37, SEQ ID NO: 41 and SEQ ID NO: 45, SEQ ID NO: 49 and SEQ ID NO: 53, SEQ ID NO: 57 and SEQ ID NO: 61, SEQ ID NO: 65 and SEQ ID NO: 69, SEQ ID NO: 73 and SEQ ID NO: 77, SEQ ID NO: 81 and SEQ ID NO: 85, SEQ ID NO: 89 and SEQ ID NO: 93, SEQ ID NO: 97 and SEQ ID NO: 101, SEQ ID NO: 105 and SEQ ID NO: 109, SEQ ID NO: 113 and SEQ ID NO: 117, SEQ ID NO: 121 and SEQ ID NO: 125, SEQ ID NO: 129 and SEQ ID NO: 133, SEQ ID NO: 137 and SEQ ID NO: 141, SEQ ID NO: 145 and SEQ ID NO: 149, SEQ ID NO: 153 and SEQ ID NO: 157, SEQ ID NO: 161 and SEQ ID NO: 165, SEQ ID NO: 169 and SEQ ID NO: 173, SEQ ID NO: 177 and SEQ ID NO: 181, SEQ ID NO: 185 and SEQ ID NO: 189, SEQ ID NO: 203 and SEQ ID NO: 207, SEQ ID NO: 211 and SEQ ID NO: 215, SEQ ID NO: 219 and SEQ ID NO: 223, SEQ ID NO: 227 and SEQ ID NO: 231, SEQ ID NO: 235 and SEQ ID NO: 239, SEQ ID NO: 243 and SEQ ID NO: 247, SEQ ID NO: 251 and SEQ ID NO: 255, SEQ ID NO: 259 and SEQ ID NO: 263, SEQ ID NO: 267 and SEQ ID NO: 271, SEQ ID NO: 275 and SEQ ID NO: 279, SEQ ID NO: 283 and SEQ ID NO: 287, SEQ ID NO: 291 and SEQ ID NO: 295, SEQ ID NO: 299 and SEQ ID NO: 303, SEQ ID NO: 307 and SEQ ID NO: 311, SEQ ID NO: 315 and SEQ ID NO: 319, SEQ ID NO: 323 and SEQ ID NO: 327, SEQ ID NO: 331 and SEQ ID NO: 335, SEQ ID NO: 339 and SEQ ID NO: 343, SEQ ID NO: 347 and SEQ ID NO: 351, SEQ ID NO: 355 and SEQ ID NO: 359, SEQ ID NO: 363 and SEQ ID NO: 367, SEQ ID NO: 371 and SEQ ID NO: 375, SEQ ID NO: 379 and SEQ ID NO: 383, or SEQ ID NO: 387 and SEQ ID NO: 391.

8. An antibody or antigen-binding fragment or derivative thereof comprising an antigen-binding domain that specifically binds to glucocorticoid-induced TNF receptor (GITR), wherein the antigen-binding domain comprises a heavy chain variable region (VH) and light chain variable region (VL), wherein the VH comprises an amino acid sequence at least 80%, 85%, 90%, 95%, or 100% identical to SEQ ID NO: 1, SEQ ID NO: 9, SEQ ID NO: 17, SEQ ID NO: 25, SEQ ID NO: 33, SEQ ID NO: 41, SEQ ID NO: 49, SEQ ID NO: 57, SEQ ID NO: 65, SEQ ID NO: 73, SEQ ID NO: 81, SEQ ID NO: 89, SEQ ID NO: 97, SEQ ID NO: 105, SEQ ID NO: 113, SEQ ID NO: 121, SEQ ID NO: 129, SEQ ID NO: 137, SEQ ID NO: 145, SEQ ID NO: 153, SEQ ID NO: 161, SEQ ID NO: 169, SEQ ID NO: 177, SEQ ID NO: 185, SEQ ID NO: 203, SEQ ID NO: 211, SEQ ID NO: 219, SEQ ID NO: 227, SEQ ID NO: 235, SEQ ID NO: 243, SEQ ID NO: 251, SEQ ID NO: 259, SEQ ID NO: 267, SEQ ID NO: 275, SEQ ID NO: 283, SEQ ID NO: 291, SEQ ID NO: 299, SEQ ID NO: 307, SEQ ID NO: 315, SEQ ID NO: 323, SEQ ID NO: 331, SEQ ID NO: 339, SEQ ID NO: 347, SEQ ID NO: 355, SEQ ID NO: 363, SEQ ID NO: 371, SEQ ID NO: 379, or SEQ ID NO: 387.

9. The antibody or fragment or derivative thereof of claim 8, wherein the VL comprises an amino acid sequence at least 80%, 85%, 90%, 95%, or 100% identical to SEQ ID NO: 5, SEQ ID NO: 13, SEQ ID NO: 21, SEQ ID NO: 29, SEQ ID NO: 37, SEQ ID NO: 45, SEQ ID NO: 53, SEQ ID NO: 61, SEQ ID NO: 69, SEQ ID NO: 77, SEQ ID NO: 85, SEQ ID NO: 93, SEQ ID NO: 101, SEQ ID NO: 109, SEQ ID NO: 117, SEQ ID NO: 125, SEQ ID NO: 133, SEQ ID NO: 141, SEQ ID NO: 149, SEQ ID NO: 157, SEQ ID NO: 165, SEQ ID NO: 173, SEQ ID NO: 181, SEQ ID NO: 189, SEQ ID NO: 207, SEQ ID NO: 215, SEQ ID NO: 223, SEQ ID NO: 231, SEQ ID NO: 239, SEQ ID NO: 247, SEQ ID NO: 255, SEQ ID NO: 263, SEQ ID NO: 271, SEQ ID NO: 279, SEQ ID NO: 287, SEQ ID NO: 295, SEQ ID NO: 303, SEQ ID NO: 311, SEQ ID NO: 319, SEQ ID NO: 327, SEQ ID NO: 335, SEQ ID NO: 343, SEQ ID NO: 351, SEQ ID NO: 359, SEQ ID NO: 367, SEQ ID NO: 375, SEQ ID NO: 383, or SEQ ID NO: 391.

10. An antibody or antigen-binding fragment or derivative thereof comprising an antigen-binding domain that specifically binds to glucocorticoid-induced TNF receptor (GITR), wherein the antigen-binding domain comprises a heavy chain variable region (VH) and light chain variable region (VL), wherein the VL comprises an amino acid sequence at least 80%, 85%, 90%, 95%, or 100% identical to SEQ ID NO: 5, SEQ ID NO: 13, SEQ ID NO: 21, SEQ ID NO: 29, SEQ ID NO: 37, SEQ ID NO: 45, SEQ ID NO: 53, SEQ ID NO: 61, SEQ ID NO: 69, SEQ ID NO: 77, SEQ ID NO: 85, SEQ ID NO: 93, SEQ ID NO: 101, SEQ ID NO: 109, SEQ ID NO: 117, SEQ ID NO: 125, SEQ ID NO: 133, SEQ ID NO: 141, SEQ ID NO: 149, SEQ ID NO: 157, SEQ ID NO: 165, SEQ ID NO: 173, SEQ ID NO: 181, SEQ ID NO: 189, SEQ ID NO: 207, SEQ ID NO: 215, SEQ ID NO: 223, SEQ ID NO: 231, SEQ ID NO: 239, SEQ ID NO: 247, SEQ ID NO: 255, SEQ ID NO: 263, SEQ ID NO: 271, SEQ ID NO: 279, SEQ ID NO: 287, SEQ ID NO: 295, SEQ ID NO: 303, SEQ ID NO: 311, SEQ ID NO: 319, SEQ ID NO: 327, SEQ ID NO: 335, SEQ ID NO: 343, SEQ ID NO: 351, SEQ ID NO: 359, SEQ ID NO: 367, SEQ ID NO: 375, SEQ ID NO: 383, or SEQ ID NO: 391.

11. The antibody or fragment or derivative thereof of claim 10, wherein the VH comprises an amino acid sequence at least 80%, 85%, 90%, 95%, or 100% identical to SEQ ID NO: 1, SEQ ID NO: 9, SEQ ID NO: 17, SEQ ID NO: 25, SEQ ID NO: 33, SEQ ID NO: 41, SEQ ID NO: 49, SEQ ID NO: 57, SEQ ID NO: 65, SEQ ID NO: 73, SEQ ID NO: 81, SEQ ID NO: 89, SEQ ID NO: 97, SEQ ID NO: 105, SEQ ID NO: 113, SEQ ID NO: 121, SEQ ID NO: 129, SEQ ID NO: 137, SEQ ID NO: 145, SEQ ID NO: 153, SEQ ID NO: 161, SEQ ID NO: 169, SEQ ID NO: 177, SEQ ID NO: 185, SEQ ID NO: 203, SEQ ID NO: 211, SEQ ID NO: 219, SEQ ID NO: 227, SEQ ID NO: 235, SEQ ID NO: 243, SEQ ID NO: 251, SEQ ID NO: 259, SEQ ID NO: 267, SEQ ID NO: 275, SEQ ID NO: 283, SEQ ID NO: 291, SEQ ID NO: 299, SEQ ID NO: 307, SEQ ID NO: 315, SEQ ID NO: 323, SEQ ID NO: 331, SEQ ID NO: 339, SEQ ID NO: 347, SEQ ID NO: 355, SEQ ID NO: 363, SEQ ID NO: 371, SEQ ID NO: 379, or SEQ ID NO: 387.

12. An antibody or antigen-binding fragment or derivative thereof comprising an antigen-binding domain that specifically binds to glucocorticoid-induced TNF receptor (GITR), wherein the antigen-binding domain comprises a heavy chain variable region (VH) and light chain variable region (VL), wherein the VH and VL comprise amino acid sequences at least 80%, at least 85%, at least 90%, at least 95% or 100% identical to the mature VH and VL amino acid sequences comprising, respectively, SEQ ID NO: 1 and SEQ ID NO: 5, SEQ ID NO: 9 and SEQ ID NO: 13, SEQ ID NO: 17 and SEQ ID NO: 21, SEQ ID NO: 25 and SEQ ID NO: 29, SEQ ID NO: 33 and SEQ ID NO: 37, SEQ ID NO: 41 and SEQ ID NO: 45, SEQ ID NO: 49 and SEQ ID NO: 53, SEQ ID NO: 57 and SEQ ID NO: 61, SEQ ID NO: 65 and SEQ ID NO: 69, SEQ ID NO: 73 and SEQ ID NO: 77, SEQ ID NO: 81 and SEQ ID NO: 85, SEQ ID NO: 89 and SEQ ID NO: 93, SEQ ID NO: 97 and SEQ ID NO: 101, SEQ ID NO: 105 and SEQ ID NO: 109, SEQ ID NO: 113 and SEQ ID NO: 117, SEQ ID NO: 121 and SEQ ID NO: 125, SEQ ID NO: 129 and SEQ ID NO: 133, SEQ ID NO: 137 and SEQ ID NO: 141, SEQ ID NO: 145 and SEQ ID NO: 149, SEQ ID NO: 153 and SEQ ID NO: 157, SEQ ID NO: 161 and SEQ ID NO: 165, SEQ ID NO: 169 and SEQ ID NO: 173, SEQ ID NO: 177 and SEQ ID NO: 181, SEQ ID NO: 185 and SEQ ID NO: 189, SEQ ID NO: 203 and SEQ ID NO: 207, SEQ ID NO: 211 and SEQ ID NO: 215, SEQ ID NO: 219 and SEQ ID NO: 223, SEQ ID NO: 227 and SEQ ID NO: 231, SEQ ID NO: 235 and SEQ ID NO: 239, SEQ ID NO: 243 and SEQ ID NO: 247, SEQ ID NO: 251 and SEQ ID NO: 255, SEQ ID NO: 259 and SEQ ID NO: 263, SEQ ID NO: 267 and SEQ ID NO: 271, SEQ ID NO: 275 and SEQ ID NO: 279, SEQ ID NO: 283 and SEQ ID NO: 287, SEQ ID NO: 291 and SEQ ID NO: 295, SEQ ID NO: 299 and SEQ ID NO: 303, SEQ ID NO: 307 and SEQ ID NO: 311, SEQ ID NO: 315 and SEQ ID NO: 319, SEQ ID NO: 323 and SEQ ID NO: 327, SEQ ID NO: 331 and SEQ ID NO: 335, SEQ ID NO: 339 and SEQ ID NO: 343, SEQ ID NO: 347 and SEQ ID NO: 351, SEQ ID NO: 355 and SEQ ID NO: 359, SEQ ID NO: 363 and SEQ ID NO: 367, SEQ ID NO: 371 and SEQ ID NO: 375, SEQ ID NO: 379 and SEQ ID NO: 383, or SEQ ID NO: 387 and SEQ ID NO: 391.

13. The antibody or fragment or derivative thereof of any one of claims 1 to 12, which is an Fv fragment, a single-chain Fv fragment (scFv), or a disulfide-linked Fv fragment (sdFv).

14. The antibody or fragment or derivative thereof of any one of claims 1 to 12, which comprises a single bivalent binding unit comprising two antigen-binding domains wherein at least one antigen-binding domain specifically binds to GITR, wherein the binding unit comprises two heavy chains each comprising a heavy chain constant region or fragment or variant thereof, and wherein at least one heavy chain constant region or variant thereof of the binding unit is fused to a copy of the VH.

15. The antibody or fragment or derivative thereof of claim 14, wherein both heavy chain constant regions or fragments or variants thereof of the binding unit are fused to a copy of the VH.

16. The antibody or fragment or derivative thereof of claim 14 or claim 15, wherein the heavy chains comprise IgG heavy chain constant regions or fragments or variants thereof.

17. The antibody or fragment or derivative thereof of any one of claims 14 to 16, wherein the binding unit further comprises two light chains each comprising a light chain constant region or fragment or variant thereof, and wherein at least one light chain constant region is fused to a copy of the VL.

18. The antibody or fragment or derivative thereof of claim 17, wherein both light chain constant regions or fragments or variants thereof of the binding unit are fused to a copy of the VL.

19. The antibody or fragment or derivative thereof of any one of claims 14 to 17, comprising a complete antibody, an Fab fragment, an Fab′ fragment, or an F(ab′)₂ fragment.

20. The antibody or fragment or derivative thereof of any one of claims 14 to 19, which is a human antibody, fragment, or derivative thereof.

21. The antibody or fragment or derivative thereof of any one of claims 1 to 12, which is a multimeric antibody comprising two, five, or six bivalent binding units and four, ten, or twelve antigen-binding domains wherein at least one, two, three, four, five, six, seven, eight, nine, ten, eleven, or twelve antigen-binding domains specifically binds to GITR;
wherein each binding unit comprises two heavy chains each comprising an IgA or IgM constant region or a multimerizing fragment or variant thereof, and wherein at least one heavy chain constant region of the binding unit is fused to a copy of the VH.

22. The antibody or fragment or derivative thereof of claim 21, which is dimeric and comprises two bivalent IgA binding units and a J chain or fragment or variant thereof, wherein each binding unit comprises two IgA heavy chain constant regions or multimerizing fragments or variants thereof.

23. The antibody or fragment or derivative thereof of claim 22, further comprising a secretory component, or fragment or variant thereof.

24. The antibody or fragment or derivative thereof of claim 22 or claim 23, wherein the IgA heavy chain constant regions or fragments or variants thereof each comprise a Cα3-tp domain.

25. The antibody or fragment or derivative thereof of claim 24, wherein the IgA heavy chain constant regions or fragments or variants thereof each further comprise a Cα1 domain, a Cα2 domain, an IgA hinge region, or any combination thereof.

26. The antibody or fragment or derivative thereof of claim 21, which is hexameric or pentameric and comprises five or six bivalent IgM binding units, wherein each binding unit comprises two IgM heavy chain constant regions or multimerizing fragments or variants thereof.

27. The antibody or fragment or derivative thereof of claim 26, wherein the IgM heavy chain constant regions or fragments or variants thereof each comprise a Cμ4-tp domain or fragment or variant thereof.

28. The antibody or fragment or derivative thereof of claim 27, wherein the IgM heavy chain constant regions or fragments or variants thereof each further comprise a Cμ1 domain, a Cμ2 domain, a Cμ3 domain, or any combination thereof.

29. The antibody or fragment or derivative thereof of any one of claims 26 to 28 which is pentameric, and further comprises a J chain, or fragment thereof, or variant thereof.

30. The antibody or fragment or derivative thereof of any one of claims 21 to 29, wherein each binding unit further comprises two light chains each comprising a light chain constant region or fragment or variant thereof, and wherein at least one, two, three, four, five, six, seven eight, nine, ten, eleven, or twelve light chain constant regions are fused to a copy of the VL.

31. The antibody or fragment or derivative thereof of any one of claims 21 to 30, which is a human antibody, fragment, or derivative thereof.

32. The antibody or fragment or derivative thereof of any one of claims 1 to 31, which is multispecific.

33. The antibody or fragment or derivative thereof of any one of claims 1 to 32, which can specifically bind to human GITR, mouse GITR, or non-human primate GITR.

34. The antibody or fragment or derivative thereof of claim 33, wherein the non-human primate GITR is cynomolgus monkey GITR.

35. The antibody or fragment or derivative thereof of claim 33 or claim 34, which specifically binds to GITR with an affinity characterized by a dissociation constant KD no greater than 500 nM, 100 nM, 50.0 nM, 40.0 nM, 30.0 nM, 20.0 nM, 10.0 nM, 9.0 nM, 8.0 nM, 7.0 nM, 6.0 nM, 5.0 nM, 4.0 nM, 3.0 nM, 2.0 nM, 1.0 nM, 0.50 nM, 0.10 nM, 0.050 nM, 0.01 nM. 0.005 nM, or 0.001 nM; and wherein the GITR is human GITR, mouse GITR, cynomolgus monkey GITR, or any combination thereof.

36. A composition comprising the antibody or fragment or derivative thereof of any one of claims 1 to 35.

37. A polynucleotide comprising a nucleic acid sequence that encodes the antibody or fragment or derivative thereof of any one of claims 1 to 35 or a subunit thereof.

38. A vector comprising the polynucleotide of claim 37.

39. A host cell comprising the vector of claim 38.

40. A method of producing the antibody or fragment or derivative thereof of any one of claims 1 to 35, comprising culturing the host cell of claim 39, and recovering the antibody or fragment or derivative thereof.

41. A method of inducing GITR-mediated activation in a GITR-expressing cell, comprising contacting the GITR-expressing cell with the antibody or fragment or derivative thereof of any one of claims 1 to 35.

42. A method of inducing GITR translocation and clustering in GITR-expressing T cells, comprising contacting GITR-expressing T cells with the antibody or fragment or derivative thereof of any one of claims 1 to 35.

43. A method of treating cancer comprising administering to a subject in need of treatment an effective amount of the antibody or fragment or derivative thereof of any one of claims 1 to 35, wherein the antibody or fragment or derivative thereof can activate GITR-expressing CTL cells thereby triggering a tumoricidal CTL response.

44. The method of claim 43, wherein the subject is human.
